SEQUENCE LISTING (297 sequences)

SEQ ID NO: 1 - RNA, 41 nt, Artificial Sequence
  Description of Artificial Sequence Synthetic oligonucleotide
uaauuucuacucuuguagaucguuaaucgcguauaauacgg

SEQ ID NO: 2 - RNA, 40 nt, Artificial Sequence
  Description of Artificial Sequence Synthetic oligonucleotide
uaauuucuacucuuguagaucgucgccguccagcucgacc

SEQ ID NO: 3 - RNA, 40 nt, Artificial Sequence
  Description of Artificial Sequence Synthetic oligonucleotide
uaauuucuacucuuguagaucucagggcggacugggugcu

SEQ ID NO: 4 - RNA, 40 nt, Artificial Sequence; identical to SEQ ID NO: 3
  Description of Artificial Sequence Synthetic oligonucleotide

SEQ ID NO: 5 - DNA, 47 bp, Artificial Sequence
  Description of Artificial Sequence Synthetic oligonucleotide
  Description of Combined DNA/RNA Molecule Synthetic oligonucleotide
uaauuucuacucuuguagaucucagggcggacugggugcutattatt

SEQ ID NO: 6 - RNA, 40 nt, Artificial Sequence
  Description of Artificial Sequence Synthetic oligonucleotide
uaauuucuacuguuguagaucucagggcggacugggugcu

SEQ ID NO: 7 - DNA, 47 bp, Artificial Sequence
  Description of Artificial Sequence Synthetic oligonucleotide
  Description of Combined DNA/RNA Molecule Synthetic oligonucleotide
uaauuucuacuguuguagaucucagggcggacugggugcutattatt

SEQ ID NO: 8 - RNA, 40 nt, Artificial Sequence
  Description of Artificial Sequence Synthetic oligonucleotide
uaauuucuacuguuguagauucaccccugccauugagaug

SEQ ID NO: 9 - RNA, 42 nt, Artificial Sequence
  Description of Artificial Sequence Synthetic oligonucleotide
uaauuucuacuaaguguagaucguuaaucgcguauaauacgg

SEQ ID NO: 10 - RNA, 41 nt, Artificial Sequence
  Description of Artificial Sequence Synthetic oligonucleotide
uaauuucuacuaaguguagaucgucgccguccagcucgacc

SEQ ID NO: 11 - RNA, 41 nt, Artificial Sequence
  Description of Artificial Sequence Synthetic oligonucleotide
uaauuucuacuaaguguagaucucagggcggacugggugcu

SEQ ID NO: 12 - RNA, 41 nt, Artificial Sequence
  Description of Artificial Sequence Synthetic oligonucleotide
uaauuucuacuaaguguagauucaccccugccauugagaug

SEQ ID NO: 13 - DNA, 48 bp, Artificial Sequence
  Description of Artificial Sequence Synthetic oligonucleotide
  Description of Combined DNA/RNA Molecule Synthetic oligonucleotide
uaauuucuacuaaguguagauucaccccugccauugagaugtattatt

SEQ ID NO: 14 - RNA, 41 nt, Artificial Sequence
  Description of Artificial Sequence Synthetic oligonucleotide
uaauuucuacuaaguguagauccuuggugggugcuacuccu

SEQ ID NO: 15 - DNA, 48 bp, Artificial Sequence
  Description of Artificial Sequence Synthetic oligonucleotide
  Description of Combined DNA/RNA Molecule Synthetic oligonucleotide
uaauuucuacuaaguguagauccuuggugggugcuacuccutattatt

SEQ ID NO: 16 - RNA, 41 nt, Artificial Sequence
  Description of Artificial Sequence Synthetic oligonucleotide
uaauuucuacuaaguguagauugcucaugaugcacggucua

SEQ ID NO: 17 - DNA, 48 bp, Artificial Sequence
  Description of Artificial Sequence Synthetic oligonucleotide
  Description of Combined DNA/RNA Molecule Synthetic oligonucleotide
uaauuucuacuaaguguagauugcucaugaugcacggucuatattatt

SEQ ID NO: 18 - RNA, 41 nt, Artificial Sequence
  Description of Artificial Sequence Synthetic oligonucleotide
uaauuucuacuaaguguagauguggacccucagauucaacu

SEQ ID NO: 19 - DNA, 48 bp, Artificial Sequence
  Description of Artificial Sequence Synthetic oligonucleotide
  Description of Combined DNA/RNA Molecule Synthetic oligonucleotide
uaauuucuacuaaguguagauguggacccucagauucaacutattatt

SEQ ID NO: 20 - RNA, 41 nt, Artificial Sequence
  Description of Artificial Sequence Synthetic oligonucleotide
uaauuucuacuaaguguagaucccccagcgcuucagcguuc

SEQ ID NO: 21 - DNA, 48 bp, Artificial Sequence
  Description of Artificial Sequence Synthetic oligonucleotide
  Description of Combined DNA/RNA Molecule Synthetic oligonucleotide
uaauuucuacuaaguguagaucccccagcgcuucagcguuctattatt

SEQ ID NO: 22 - DNA, 48 bp, Artificial Sequence
  Description of Artificial Sequence Synthetic oligonucleotide
  Description of Combined DNA/RNA Molecule Synthetic oligonucleotide
uaauuucuacuaaguguagaucucagggcggacugggugcutattatt

SEQ ID NO: 23 - DNA, 54 bp, Artificial Sequence
  Description of Artificial Sequence Synthetic oligonucleotide
  Description of Combined DNA/RNA Molecule Synthetic oligonucleotide
uaauuucuacuaaguguagaucucagggcggacugggugcutattattattatt

SEQ ID NO: 24 - DNA, 60 bp, Artificial Sequence
  Description of Artificial Sequence Synthetic oligonucleotide
  Description of Combined DNA/RNA Molecule Synthetic oligonucleotide
uaauuucuacuaaguguagaucucagggcggacugggugcutattattattattattatt

SEQ ID NO: 25 - DNA, 72 bp, Artificial Sequence
  Description of Artificial Sequence Synthetic oligonucleotide
  Description of Combined DNA/RNA Molecule Synthetic oligonucleotide
uaauuucuacuaaguguagaucucagggcggacugggugcutattattattattattattattattattatt

SEQ ID NO: 26 - RNA, 48 nt, Artificial Sequence
  Description of Artificial Sequence Synthetic oligonucleotide
uaauuucuacuaaguguagaucucagggcggacugggugcucgccgcc

SEQ ID NO: 27 - DNA, 48 bp, Artificial Sequence
  Description of Artificial Sequence Synthetic oligonucleotide
  Description of Combined DNA/RNA Molecule Synthetic oligonucleotide
ttattatuaauuucuacuaaguguagaucucagggcggacugggugcu

SEQ ID NO: 28 - DNA, 47 bp, Artificial Sequence
  Description of Artificial Sequence Synthetic oligonucleotide
  Description of Combined DNA/RNA Molecule Synthetic oligonucleotide
ttattataauuucuacuaaguguagaucucagggcggacugggugcu

SEQ ID NO: 29 - DNA, 54 bp, Artificial Sequence
  Description of Artificial Sequence Synthetic oligonucleotide
  Description of Combined DNA/RNA Molecule Synthetic oligonucleotide
ttattattattatuaauuucuacuaaguguagaucucagggcggacugggugcu

SEQ ID NO: 30 - DNA, 60 bp, Artificial Sequence
  Description of Artificial Sequence Synthetic oligonucleotide
  Description of Combined DNA/RNA Molecule Synthetic oligonucleotide
ttattattattattattatuaauuucuacuaaguguagaucucagggcggacugggugcu

SEQ ID NO: 31 - DNA, 72 bp, Artificial Sequence
  Description of Artificial Sequence Synthetic oligonucleotide
  Description of Combined DNA/RNA Molecule Synthetic oligonucleotide
ttattattattattattattattattattatuaauuucuacuaaguguagaucucagggcggacugggugcu

SEQ ID NO: 32 - DNA, 48 bp, Artificial Sequence; identical to SEQ ID NO: 22
  Description of Artificial Sequence Synthetic oligonucleotide
  Description of Combined DNA/RNA Molecule Synthetic oligonucleotide

SEQ ID NO: 33 - DNA, 54 bp, Artificial Sequence; identical to SEQ ID NO: 23
  Description of Artificial Sequence Synthetic oligonucleotide
  Description of Combined DNA/RNA Molecule Synthetic oligonucleotide

SEQ ID NO: 34 - DNA, 60 bp, Artificial Sequence; identical to SEQ ID NO: 24
  Description of Artificial Sequence Synthetic oligonucleotide
  Description of Combined DNA/RNA Molecule Synthetic oligonucleotide

SEQ ID NO: 35 - DNA, 48 bp, Artificial Sequence; identical to SEQ ID NO: 22
  Description of Artificial Sequence Synthetic oligonucleotide
  Description of Combined DNA/RNA Molecule Synthetic oligonucleotide

SEQ ID NO: 36 - DNA, 48 bp, Artificial Sequence; identical to SEQ ID NO: 22
  Description of Artificial Sequence Synthetic oligonucleotide
  Description of Combined DNA/RNA Molecule Synthetic oligonucleotide

SEQ ID NO: 37 - DNA, 48 bp, Artificial Sequence; identical to SEQ ID NO: 22
  Description of Artificial Sequence Synthetic oligonucleotide
  Description of Combined DNA/RNA Molecule Synthetic oligonucleotide

SEQ ID NO: 38 - DNA, 48 bp, Artificial Sequence; identical to SEQ ID NO: 22
  Description of Artificial Sequence Synthetic oligonucleotide
  Description of Combined DNA/RNA Molecule Synthetic oligonucleotide

SEQ ID NO: 39 - DNA, 48 bp, Artificial Sequence; identical to SEQ ID NO: 22
  Description of Artificial Sequence Synthetic oligonucleotide
  Description of Combined DNA/RNA Molecule Synthetic oligonucleotide

SEQ ID NO: 40 - DNA, 48 bp, Artificial Sequence; identical to SEQ ID NO: 22
  Description of Artificial Sequence Synthetic oligonucleotide
  Description of Combined DNA/RNA Molecule Synthetic oligonucleotide

SEQ ID NO: 41 - DNA, 48 bp, Artificial Sequence; identical to SEQ ID NO: 27
  Description of Artificial Sequence Synthetic oligonucleotide
  Description of Combined DNA/RNA Molecule Synthetic oligonucleotide

SEQ ID NO: 42 - DNA, 54 bp, Artificial Sequence; identical to SEQ ID NO: 29
  Description of Artificial Sequence Synthetic oligonucleotide
  Description of Combined DNA/RNA Molecule Synthetic oligonucleotide

SEQ ID NO: 43 - DNA, 60 bp, Artificial Sequence; identical to SEQ ID NO: 30
  Description of Artificial Sequence Synthetic oligonucleotide
  Description of Combined DNA/RNA Molecule Synthetic oligonucleotide

SEQ ID NO: 44 - RNA, 48 nt, Artificial Sequence
  Description of Artificial Sequence Synthetic oligonucleotide
uaauuucuacuaaguguagaucucagggcggacugggugcuuauuauu

SEQ ID NO: 45 - RNA, 54 nt, Artificial Sequence
  Description of Artificial Sequence Synthetic oligonucleotide
uaauuucuacuaaguguagaucucagggcggacugggugcuuauuauuauuauu

SEQ ID NO: 46 - RNA, 60 nt, Artificial Sequence
  Description of Artificial Sequence Synthetic oligonucleotide
uaauuucuacuaaguguagaucucagggcggacugggugcuuauuauuauuauuauuauu

SEQ ID NO: 47 - RNA, 48 nt, Artificial Sequence
  Description of Artificial Sequence Synthetic oligonucleotide
uuauuauuaauuucuacuaaguguagaucucagggcggacugggugcu

SEQ ID NO: 48 - RNA, 54 nt, Artificial Sequence
  Description of Artificial Sequence Synthetic oligonucleotide
uuauuauuauuauuaauuucuacuaaguguagaucucagggcggacugggugcu

SEQ ID NO: 49 - RNA, 60 nt, Artificial Sequence
  Description of Artificial Sequence Synthetic oligonucleotide
uuauuauuauuauuauuauuaauuucuacuaaguguagaucucagggcggacugggugcu

SEQ ID NO: 50 - DNA, 40 bp, Homo sapiens
agactacagacatctcaatggcaggggtgagaaataagaa

SEQ ID NO: 51 - DNA, 40 bp, Artificial Sequence
  Description of Artificial Sequence Synthetic oligonucleotide
ttcttatttctcacccctgccattgagatgtctgtagtct

SEQ ID NO: 52 - DNA, 13 bp, Artificial Sequence
  Description of Artificial Sequence Synthetic oligonucleotide
tattattattatt

SEQ ID NO: 53 - DNA, 250 bp, Homo sapiens
caagataaataagtgaagagctagtccgctgtgagtctcctcagtgacacagggctggatcaccatcgacggcactttctgagtactcagtgcagcaaagaaagactacagacatctcaatggcaggggtgagaaataagaaaggctgctgactttaccatctgaggccacacatctgctgaaatggagataattaacatcactagaaacagcaagatgacaatataatgtctaagtagtgacatgtttt

SEQ ID NO: 54 - RNA, 60 nt, Human immunodeficiency virus 1
uucucucugcaccacucuucucuuugccuuggugggugcuacuccuaaugguucaauuuu

SEQ ID NO: 55 - DNA, 60 bp, Human immunodeficiency virus
aaaattgaaccattaggagtagcacccaccaaggcaaagagaagagtggtgcagagagaa

SEQ ID NO: 56 - DNA, 60 bp, Human immunodeficiency virus
ttctctctgcaccactcttctctttgccttggtgggtgctactcctaatggttcaatttt

SEQ ID NO: 57 - RNA, 86 nt, Hepatitis C virus
gccuugugguacugccugauagggugcuugcgagugccccgggaggucucguagaccgugcaucaugagcacaaauccuaaaccuc

SEQ ID NO: 58 - DNA, 125 bp, Artificial Sequence
  Description of Artificial Sequence Synthetic polynucleotide
cctctaatacgactcactataggcgttgggttgcgaacggccttgtggtactgcctgatagggtgcttgcgagtgccccgggaggtctcgtagaccgtgcatcatgagcacaaatcctaaacctc

SEQ ID NO: 59 - DNA, 125 bp, Artificial Sequence
  Description of Artificial Sequence Synthetic polynucleotide
gaggtttaggatttgtgctcatgatgcacggtctacgagacctcccggggcactcgcaagcaccctatcaggcagtaccacaaggccgttcgcaacccaacgcctatagtgagtcgtattagagg

SEQ ID NO: 60 - DNA, 40 bp, Artificial Sequence
  Description of Artificial Sequence Synthetic primer
ggggtctttgctcagggcggactgggtgctcaggtagtgg

SEQ ID NO: 61 - DNA, 40 bp, Artificial Sequence
  Description of Artificial Sequence Synthetic primer
ccactacctgagcacccagtccgccctgagcaaagacccc

SEQ ID NO: 62 - DNA, 40 bp, Artificial Sequence
  Description of Artificial Sequence Synthetic primer
  Description of Combined DNA/RNA Molecule Synthetic primer
ccactacctgagcacccaguccgcccugagcaaagacccc

SEQ ID NO: 63 - DNA, 40 bp, Artificial Sequence
  Description of Artificial Sequence Synthetic primer
  Description of Combined DNA/RNA Molecule Synthetic primer
ggggtctttgcucagggcggacugggugcucaggtagtgg

SEQ ID NO: 64 - DNA, 23 bp, Artificial Sequence
  Description of Artificial Sequence Synthetic primer
aaaattgaaccattaggagtagc

SEQ ID NO: 65 - DNA, 17 bp, Artificial Sequence
  Description of Artificial Sequence Synthetic primer
aaaattgaaccattagg

SEQ ID NO: 66 - DNA, 20 bp, Artificial Sequence
  Description of Artificial Sequence Synthetic primer
ttctctctgcaccactcttc

SEQ ID NO: 67 - DNA, 22 bp, Artificial Sequence
  Description of Artificial Sequence Synthetic primer
gaggtttaggatttgtgctcat

SEQ ID NO: 68 - DNA, 24 bp, Artificial Sequence
  Description of Artificial Sequence Synthetic primer
gaggtttaggatttgtgctcatga

SEQ ID NO: 69 - DNA, 19 bp, Artificial Sequence
  Description of Artificial Sequence Synthetic primer
cctctaatacgactcacta

SEQ ID NO: 70 - DNA, 16 bp, Artificial Sequence
  Description of Artificial Sequence Synthetic primer
cctctaatacgactca

SEQ ID NO: 71 - DNA, 20 bp, Artificial Sequence
  Description of Artificial Sequence Synthetic primer
gcgttgggttgcgaacggcc

SEQ ID NO: 72 - DNA, 18 bp, Artificial Sequence
  Description of Artificial Sequence Synthetic primer
gcgttgggttgcgaacgg

SEQ ID NO: 73 - DNA, 20 bp, Artificial Sequence
  Description of Artificial Sequence Synthetic primer
gccttgtggtactgcctgat

SEQ ID NO: 74 - DNA, 20 bp, Artificial Sequence
  Description of Artificial Sequence Synthetic primer
gaccccaaaatcagcgaaat

SEQ ID NO: 75 - DNA, 24 bp, Artificial Sequence
  Description of Artificial Sequence Synthetic primer
tctggttactgccagttgaatctg

SEQ ID NO: 76 - DNA, 42 bp, Artificial Sequence
  Description of Artificial Sequence Synthetic primer
cctctaatacgactcactataggaccccaaaatcagcgaaat

SEQ ID NO: 77 - DNA, 47 bp, Artificial Sequence
  Description of Artificial Sequence Synthetic primer
cctctaatacgactcactataggtctggttactgccagttgaatctg

SEQ ID NO: 78 - DNA, 22 bp, Artificial Sequence
  Description of Artificial Sequence Synthetic primer
gggagccttgaatacaccaaaa

SEQ ID NO: 79 - DNA, 21 bp, Artificial Sequence
  Description of Artificial Sequence Synthetic primer
tgtagcacgattgcagcattg

SEQ ID NO: 80 - DNA, 20 bp, Artificial Sequence
  Description of Artificial Sequence Synthetic primer
ttacaaacattggccgcaaa

SEQ ID NO: 81 - DNA, 18 bp, Artificial Sequence
  Description of Artificial Sequence Synthetic primer
gcgcgacattccgaagaa

SEQ ID NO: 82 - DNA, 22 bp, Artificial Sequence
  Description of Artificial Sequence Synthetic primer
atgtctgataatggaccccaaa

SEQ ID NO: 83 - DNA, 20 bp, Artificial Sequence
  Description of Artificial Sequence Synthetic primer
ttaagcctgggttgaatcag

SEQ ID NO: 84 - DNA, 20 bp, Artificial Sequence
  Description of Artificial Sequence Synthetic primer
cacctcgtgctgtttccttt

SEQ ID NO: 85 - DNA, 20 bp, Artificial Sequence
  Description of Artificial Sequence Synthetic primer
atcattggaccaggctgaac

SEQ ID NO: 86 - DNA, 18 bp, Artificial Sequence
  Description of Artificial Sequence Synthetic primer
tcatgacgttcgtgttgt

SEQ ID NO: 87 - DNA, 18 bp, Artificial Sequence
  Description of Artificial Sequence Synthetic primer
ttgagtgagagcggtgaa

SEQ ID NO: 88 - DNA, 42 bp, Artificial Sequence
  Description of Artificial Sequence Synthetic primer
taatgcggggtgcatttcgagatttcatctaaacgaacaaac

SEQ ID NO: 89 - DNA, 42 bp, Artificial Sequence
  Description of Artificial Sequence Synthetic primer
taaccagaatggagaacgcaagtattattgggtaaaccttgg

SEQ ID NO: 90 - DNA, 19 bp, Artificial Sequence
  Description of Artificial Sequence Synthetic primer
ctgattttggggtccatta

SEQ ID NO: 91 - DNA, 21 bp, Artificial Sequence
  Description of Artificial Sequence Synthetic primer
gtggggcgcgatcaaaacaac

SEQ ID NO: 92 - DNA, 18 bp, Artificial Sequence
  Description of Artificial Sequence Synthetic primer
gctgctgaggcttctaag

SEQ ID NO: 93 - DNA, 21 bp, Artificial Sequence
  Description of Artificial Sequence Synthetic primer
gcgtcaatatgcttattcagc

SEQ ID NO: 94 - DNA, 40 bp, Artificial Sequence
  Description of Artificial Sequence Synthetic primer
gcggccaatgtttgtaatcagtagacgtggtccagaacaa

SEQ ID NO: 95 - DNA, 39 bp, Artificial Sequence
  Description of Artificial Sequence Synthetic primer
tcagcgttcttcggaatgtcgctgtgtaggtcaaccacg

SEQ ID NO: 96 - DNA, 22 bp, Artificial Sequence
  Description of Artificial Sequence Synthetic primer
ccttgtctgattagttcctggt

SEQ ID NO: 97 - DNA, 18 bp, Artificial Sequence
  Description of Artificial Sequence Synthetic primer
tggcatggaagtcacacc

SEQ ID NO: 98 - DNA, 10 bp, Artificial Sequence
  Description of Artificial Sequence Synthetic oligonucleotide
tattattatt

SEQ ID NO: 99 - DNA, 48 bp, Artificial Sequence; identical to SEQ ID NO: 22
  Description of Artificial Sequence Synthetic oligonucleotide
  Description of Combined DNA/RNA Molecule Synthetic oligonucleotide

SEQ ID NO: 100 - RNA, 41 nt, Artificial Sequence; identical to SEQ ID NO: 11
  Description of Artificial Sequence Synthetic oligonucleotide

SEQ ID NO: 101 - RNA, 41 nt, Artificial Sequence; identical to SEQ ID NO: 11
  Description of Artificial Sequence Synthetic oligonucleotide

SEQ ID NO: 102 - DNA, 54 bp, Artificial Sequence; identical to SEQ ID NO: 23
  Description of Artificial Sequence Synthetic oligonucleotide
  Description of Combined DNA/RNA Molecule Synthetic oligonucleotide

SEQ ID NO: 103 - DNA, 54 bp, Artificial Sequence; identical to SEQ ID NO: 23
  Description of Artificial Sequence Synthetic oligonucleotide
  Description of Combined DNA/RNA Molecule Synthetic oligonucleotide

SEQ ID NO: 104 - DNA, 60 bp, Artificial Sequence; identical to SEQ ID NO: 30
  Description of Artificial Sequence Synthetic oligonucleotide
  Description of Combined DNA/RNA Molecule Synthetic oligonucleotide

SEQ ID NO: 105 - DNA, 60 bp, Artificial Sequence; identical to SEQ ID NO: 30
  Description of Artificial Sequence Synthetic oligonucleotide
  Description of Combined DNA/RNA Molecule Synthetic oligonucleotide

SEQ ID NO: 106 - DNA, 54 bp, Artificial Sequence; identical to SEQ ID NO: 23
  Description of Artificial Sequence Synthetic oligonucleotide
  Description of Combined DNA/RNA Molecule Synthetic oligonucleotide

SEQ ID NO: 107 - DNA, 22 bp, Artificial Sequence
  Description of Artificial Sequence Synthetic primer
aataataagcacccagtccgcc

SEQ ID NO: 108 - DNA, 21 bp, Artificial Sequence
  Description of Artificial Sequence Synthetic primer
aataataagcacccagtccgc

SEQ ID NO: 109 - DNA, 20 bp, Artificial Sequence
  Description of Artificial Sequence Synthetic primer
aataataagcacccagtccg

SEQ ID NO: 110 - DNA, 19 bp, Artificial Sequence
  Description of Artificial Sequence Synthetic primer
aataataagcacccagtcc

SEQ ID NO: 111 - DNA, 18 bp, Artificial Sequence
  Description of Artificial Sequence Synthetic primer
aataataagcacccagtc

SEQ ID NO: 112 - DNA, 17 bp, Artificial Sequence
  Description of Artificial Sequence Synthetic primer
aataataagcacccagt

SEQ ID NO: 113 - DNA, 16 bp, Artificial Sequence
  Description of Artificial Sequence Synthetic primer
aataataagcacccag

SEQ ID NO: 114 - DNA, 15 bp, Artificial Sequence
  Description of Artificial Sequence Synthetic primer
aataataagcaccca

SEQ ID NO: 115 - DNA, 14 bp, Artificial Sequence
  Description of Artificial Sequence Synthetic primer
aataataagcaccc

SEQ ID NO: 116 - DNA, 13 bp, Artificial Sequence
  Description of Artificial Sequence Synthetic primer
aataataagcacc

SEQ ID NO: 117 - DNA, 12 bp, Artificial Sequence
  Description of Artificial Sequence Synthetic primer
aataataagcac

SEQ ID NO: 118 - DNA, 11 bp, Artificial Sequence
  Description of Artificial Sequence Synthetic primer
aataataagca

SEQ ID NO: 119 - DNA, 15 bp, Artificial Sequence
  Description of Artificial Sequence Synthetic primer
agcacccagtccgcc

SEQ ID NO: 120 - DNA, 14 bp, Artificial Sequence
  Description of Artificial Sequence Synthetic primer
agcacccagtccgc

SEQ ID NO: 121 - DNA, 13 bp, Artificial Sequence
  Description of Artificial Sequence Synthetic primer
agcacccagtccg

SEQ ID NO: 122 - DNA, 12 bp, Artificial Sequence
  Description of Artificial Sequence Synthetic primer
agcacccagtcc

SEQ ID NO: 123 - DNA, 11 bp, Artificial Sequence
  Description of Artificial Sequence Synthetic primer
agcacccagtc

SEQ ID NO: 124 - DNA, 10 bp, Artificial Sequence
  Description of Artificial Sequence Synthetic primer
agcacccagt

SEQ ID NO: 125 - DNA, 30 bp, Artificial Sequence
  Description of Artificial Sequence Synthetic primer
agtactcagtgcagcaaagaaagactacag

SEQ ID NO: 126 - DNA, 33 bp, Artificial Sequence
  Description of Artificial Sequence Synthetic primer
acattatattgtcatcttgctgtttctagtgat

SEQ ID NO: 127 - DNA, 30 bp, Artificial Sequence
  Description of Artificial Sequence Synthetic primer
agtgaagagctagtccgctgtgagtctcct

SEQ ID NO: 128 - DNA, 32 bp, Artificial Sequence
  Description of Artificial Sequence Synthetic primer
ctgtttctagtgatgttaattatctccatttc

SEQ ID NO: 129 - DNA, 30 bp, Artificial Sequence
  Description of Artificial Sequence Synthetic primer
aagagctagtccgctgtgagtctcctcagt

SEQ ID NO: 130 - DNA, 32 bp, Artificial Sequence
  Description of Artificial Sequence Synthetic primer
gtttctagtgatgttaattatctccatttcag

SEQ ID NO: 131 - DNA, 42 bp, Artificial Sequence
  Description of Artificial Sequence Synthetic primer
cctctaatacgactcactataggaacggcatcaaggtgaact

SEQ ID NO: 132 - DNA, 43 bp, Artificial Sequence
  Description of Artificial Sequence Synthetic primer
cctctaatacgactcactataggcgaccactaccagcagaaca

SEQ ID NO: 133 - DNA, 20 bp, Artificial Sequence
  Description of Artificial Sequence Synthetic primer
acttcaagatccgccacaac

SEQ ID NO: 134 - DNA, 20 bp, Artificial Sequence
  Description of Artificial Sequence Synthetic primer
gaacggcatcaaggtgaact

SEQ ID NO: 135 - DNA, 20 bp, Artificial Sequence
  Description of Artificial Sequence Synthetic primer
cgaccactaccagcagaaca

SEQ ID NO: 136 - DNA, 40 bp, Artificial Sequence; identical to SEQ ID NO: 61
  Description of Artificial Sequence Synthetic oligonucleotide

SEQ ID NO: 137 - DNA, 40 bp, Artificial Sequence; identical to SEQ ID NO: 60
  Description of Artificial Sequence Synthetic oligonucleotide

SEQ ID NO: 138 - DNA, 40 bp, Artificial Sequence; identical to SEQ ID NO: 60
  Description of Artificial Sequence Synthetic oligonucleotide

SEQ ID NO: 139 - DNA, 40 bp, Artificial Sequence; identical to SEQ ID NO: 61
  Description of Artificial Sequence Synthetic oligonucleotide

SEQ ID NO: 140 - DNA, 40 bp, Artificial Sequence
  Description of Artificial Sequence Synthetic oligonucleotide
ccactacctgggcacccagtccgccctgagcaaagacccc

SEQ ID NO: 141 - DNA, 40 bp, Artificial Sequence
  Description of Artificial Sequence Synthetic oligonucleotide
ccactacctgaacacccagtccgccctgagcaaagacccc

SEQ ID NO: 142 - DNA, 40 bp, Artificial Sequence
  Description of Artificial Sequence Synthetic oligonucleotide
ccactacctgagtacccagtccgccctgagcaaagacccc

SEQ ID NO: 143 - DNA, 40 bp, Artificial Sequence
  Description of Artificial Sequence Synthetic oligonucleotide
ccactacctgagcgcccagtccgccctgagcaaagacccc

SEQ ID NO: 144 - DNA, 40 bp, Artificial Sequence
  Description of Artificial Sequence Synthetic oligonucleotide
ccactacctgagcatccagtccgccctgagcaaagacccc

SEQ ID NO: 145 - DNA, 40 bp, Artificial Sequence
  Description of Artificial Sequence Synthetic oligonucleotide
ccactacctgagcactcagtccgccctgagcaaagacccc

SEQ ID NO: 146 - DNA, 40 bp, Artificial Sequence
  Description of Artificial Sequence Synthetic oligonucleotide
ccactacctgagcacctagtccgccctgagcaaagacccc

SEQ ID NO: 147 - DNA, 40 bp, Artificial Sequence
  Description of Artificial Sequence Synthetic oligonucleotide
ccactacctgagcacccggtccgccctgagcaaagacccc

SEQ ID NO: 148 - DNA, 40 bp, Artificial Sequence
  Description of Artificial Sequence Synthetic oligonucleotide
ccactacctgagcacccaatccgccctgagcaaagacccc

SEQ ID NO: 149 - DNA, 40 bp, Artificial Sequence
  Description of Artificial Sequence Synthetic oligonucleotide
ccactacctgagcacccagcccgccctgagcaaagacccc

SEQ ID NO: 150 - DNA, 40 bp, Artificial Sequence
  Description of Artificial Sequence Synthetic oligonucleotide
ccactacctgagcacccagttcgccctgagcaaagacccc

SEQ ID NO: 151 - DNA, 40 bp, Artificial Sequence
  Description of Artificial Sequence Synthetic oligonucleotide
ccactacctgagcacccagtctgccctgagcaaagacccc

SEQ ID NO: 152 - DNA, 40 bp, Artificial Sequence
  Description of Artificial Sequence Synthetic oligonucleotide
ccactacctgagcacccagtccaccctgagcaaagacccc

SEQ ID NO: 153 - DNA, 40 bp, Artificial Sequence
  Description of Artificial Sequence Synthetic oligonucleotide
ccactacctgagcacccagtccgtcctgagcaaagacccc

SEQ ID NO: 154 - DNA, 40 bp, Artificial Sequence
  Description of Artificial Sequence Synthetic oligonucleotide
ccactacctgagcacccagtccgctctgagcaaagacccc

SEQ ID NO: 155 - DNA, 40 bp, Artificial Sequence
  Description of Artificial Sequence Synthetic oligonucleotide
ccactacctgagcacccagtccgccttgagcaaagacccc

SEQ ID NO: 156 - DNA, 40 bp, Artificial Sequence
  Description of Artificial Sequence Synthetic oligonucleotide
ccactacctgagcacccagtccgccccgagcaaagacccc

SEQ ID NO: 157 - DNA, 40 bp, Artificial Sequence
  Description of Artificial Sequence Synthetic oligonucleotide
ccactacctgagcacccagtccgccctaagcaaagacccc

SEQ ID NO: 158 - DNA, 40 bp, Artificial Sequence
  Description of Artificial Sequence Synthetic oligonucleotide
ccactacctgagcacccagtccgccctgggcaaagacccc

SEQ ID NO: 159 - DNA, 40 bp, Artificial Sequence
  Description of Artificial Sequence Synthetic oligonucleotide
ccactacctgagcacccagtccgccctgaacaaagacccc

SEQ ID NO: 160 - DNA, 40 bp, Artificial Sequence
  Description of Artificial Sequence Synthetic oligonucleotide
ggggtctttgctcagggcggactgggtgcccaggtagtgg

SEQ ID NO: 161 - DNA, 40 bp, Artificial Sequence
  Description of Artificial Sequence Synthetic oligonucleotide
ggggtctttgctcagggcggactgggtgttcaggtagtgg

SEQ ID NO: 162 - DNA, 40 bp, Artificial Sequence
  Description of Artificial Sequence Synthetic oligonucleotide
ggggtctttgctcagggcggactgggtactcaggtagtgg

SEQ ID NO: 163 - DNA, 40 bp, Artificial Sequence
  Description of Artificial Sequence Synthetic oligonucleotide
ggggtctttgctcagggcggactgggcgctcaggtagtgg

SEQ ID NO: 164 - DNA, 40 bp, Artificial Sequence
  Description of Artificial Sequence Synthetic oligonucleotide
ggggtctttgctcagggcggactggatgctcaggtagtgg

SEQ ID NO: 165 - DNA, 40 bp, Artificial Sequence
  Description of Artificial Sequence Synthetic oligonucleotide
ggggtctttgctcagggcggactgagtgctcaggtagtgg

SEQ ID NO: 166 - DNA, 40 bp, Artificial Sequence
  Description of Artificial Sequence Synthetic oligonucleotide
ggggtctttgctcagggcggactaggtgctcaggtagtgg

SEQ ID NO: 167 - DNA, 40 bp, Artificial Sequence
  Description of Artificial Sequence Synthetic oligonucleotide
ggggtctttgctcagggcggaccgggtgctcaggtagtgg

SEQ ID NO: 168 - DNA, 40 bp, Artificial Sequence
  Description of Artificial Sequence Synthetic oligonucleotide
ggggtctttgctcagggcggattgggtgctcaggtagtgg

SEQ ID NO: 169 - DNA, 40 bp, Artificial Sequence
  Description of Artificial Sequence Synthetic oligonucleotide
ggggtctttgctcagggcgggctgggtgctcaggtagtgg

SEQ ID NO: 170 - DNA, 40 bp, Artificial Sequence
  Description of Artificial Sequence Synthetic oligonucleotide
ggggtctttgctcagggcgaactgggtgctcaggtagtgg

SEQ ID NO: 171 - DNA, 40 bp, Artificial Sequence
  Description of Artificial Sequence Synthetic oligonucleotide
ggggtctttgctcagggcagactgggtgctcaggtagtgg

SEQ ID NO: 172 - DNA, 40 bp, Artificial Sequence
  Description of Artificial Sequence Synthetic oligonucleotide
ggggtctttgctcagggtggactgggtgctcaggtagtgg

SEQ ID NO: 173 - DNA, 40 bp, Artificial Sequence
  Description of Artificial Sequence Synthetic oligonucleotide
ggggtctttgctcaggacggactgggtgctcaggtagtgg

SEQ ID NO: 174 - DNA, 40 bp, Artificial Sequence
  Description of Artificial Sequence Synthetic oligonucleotide
ggggtctttgctcagagcggactgggtgctcaggtagtgg

SEQ ID NO: 175 - DNA, 40 bp, Artificial Sequence
  Description of Artificial Sequence Synthetic oligonucleotide
ggggtctttgctcaaggcggactgggtgctcaggtagtgg

SEQ ID NO: 176 - DNA, 40 bp, Artificial Sequence
  Description of Artificial Sequence Synthetic oligonucleotide
ggggtctttgctcggggcggactgggtgctcaggtagtgg

SEQ ID NO: 177 - DNA, 40 bp, Artificial Sequence
  Description of Artificial Sequence Synthetic oligonucleotide
ggggtctttgcttagggcggactgggtgctcaggtagtgg

SEQ ID NO: 178 - DNA, 40 bp, Artificial Sequence
  Description of Artificial Sequence Synthetic oligonucleotide
ggggtctttgcccagggcggactgggtgctcaggtagtgg

SEQ ID NO: 179 - DNA, 40 bp, Artificial Sequence
  Description of Artificial Sequence Synthetic oligonucleotide
ggggtctttgttcagggcggactgggtgctcaggtagtgg

SEQ ID NO: 180 - DNA, 40 bp, Artificial Sequence
  Description of Artificial Sequence Synthetic oligonucleotide
ccactacctggacacccagtccgccctgagcaaagacccc

SEQ ID NO: 181 - DNA, 40 bp, Artificial Sequence
  Description of Artificial Sequence Synthetic oligonucleotide
ccactacctgaatacccagtccgccctgagcaaagacccc

SEQ ID NO: 182 - DNA, 40 bp, Artificial Sequence
  Description of Artificial Sequence Synthetic oligonucleotide
ccactacctgagtgcccagtccgccctgagcaaagacccc

SEQ ID NO: 183 - DNA, 40 bp, Artificial Sequence
  Description of Artificial Sequence Synthetic oligonucleotide
ccactacctgagcgtccagtccgccctgagcaaagacccc

SEQ ID NO: 184 - DNA, 40 bp, Artificial Sequence
  Description of Artificial Sequence Synthetic oligonucleotide
ccactacctgagcattcagtccgccctgagcaaagacccc

SEQ ID NO: 185 - DNA, 40 bp, Artificial Sequence
  Description of Artificial Sequence Synthetic oligonucleotide
ccactacctgagcacttagtccgccctgagcaaagacccc

SEQ ID NO: 186 - DNA, 40 bp, Artificial Sequence
  Description of Artificial Sequence Synthetic oligonucleotide
ccactacctgagcacctggtccgccctgagcaaagacccc

SEQ ID NO: 187 - DNA, 40 bp, Artificial Sequence
  Description of Artificial Sequence Synthetic oligonucleotide
ccactacctgagcacccgatccgccctgagcaaagacccc

SEQ ID NO: 188 - DNA, 40 bp, Artificial Sequence
  Description of Artificial Sequence Synthetic oligonucleotide
ccactacctgagcacccaacccgccctgagcaaagacccc

SEQ ID NO: 189 - DNA, 40 bp, Artificial Sequence
  Description of Artificial Sequence Synthetic oligonucleotide
ccactacctgagcacccagctcgccctgagcaaagacccc

SEQ ID NO: 190 - DNA, 40 bp, Artificial Sequence
  Description of Artificial Sequence Synthetic oligonucleotide
ccactacctgagcacccagtttgccctgagcaaagacccc

SEQ ID NO: 191 - DNA, 40 bp, Artificial Sequence
  Description of Artificial Sequence Synthetic oligonucleotide
ccactacctgagcacccagtctaccctgagcaaagacccc

SEQ ID NO: 192 - DNA, 40 bp, Artificial Sequence
  Description of Artificial Sequence Synthetic oligonucleotide
ccactacctgagcacccagtccatcctgagcaaagacccc

SEQ ID NO: 193 - DNA, 40 bp, Artificial Sequence
  Description of Artificial Sequence Synthetic oligonucleotide
ccactacctgagcacccagtccgttctgagcaaagacccc

SEQ ID NO: 194 - DNA, 40 bp, Artificial Sequence
  Description of Artificial Sequence Synthetic oligonucleotide
ccactacctgagcacccagtccgctttgagcaaagacccc

SEQ ID NO: 195 - DNA, 40 bp, Artificial Sequence
  Description of Artificial Sequence Synthetic oligonucleotide
ccactacctgagcacccagtccgcctcgagcaaagacccc

SEQ ID NO: 196 - DNA, 40 bp, Artificial Sequence
  Description of Artificial Sequence Synthetic oligonucleotide
ccactacctgagcacccagtccgccccaagcaaagacccc

SEQ ID NO: 197 - DNA, 40 bp, Artificial Sequence
  Description of Artificial Sequence Synthetic oligonucleotide
ccactacctgagcacccagtccgccctaggcaaagacccc

SEQ ID NO: 198 - DNA, 40 bp, Artificial Sequence
  Description of Artificial Sequence Synthetic oligonucleotide
ccactacctgagcacccagtccgccctggacaaagacccc

SEQ ID NO: 199 - DNA, 40 bp, Artificial Sequence
  Description of Artificial Sequence Synthetic oligonucleotide
ggggtctttgctcagggcggactgggtgtccaggtagtgg

SEQ ID NO: 200 - DNA, 40 bp, Artificial Sequence
  Description of Artificial Sequence Synthetic oligonucleotide
ggggtctttgctcagggcggactgggtattcaggtagtgg

SEQ ID NO: 201 - DNA, 40 bp, Artificial Sequence
  Description of Artificial Sequence Synthetic oligonucleotide
ggggtctttgctcagggcggactgggcactcaggtagtgg

SEQ ID NO: 202 - DNA, 40 bp, Artificial Sequence
  Description of Artificial Sequence Synthetic oligonucleotide
ggggtctttgctcagggcggactggacgctcaggtagtgg

SEQ ID NO: 203 - DNA, 40 bp, Artificial Sequence
  Description of Artificial Sequence Synthetic oligonucleotide
ggggtctttgctcagggcggactgaatgctcaggtagtgg

SEQ ID NO: 204 - DNA, 40 bp, Artificial Sequence
  Description of Artificial Sequence Synthetic oligonucleotide
ggggtctttgctcagggcggactaagtgctcaggtagtgg

SEQ ID NO: 205 - DNA, 40 bp, Artificial Sequence
  Description of Artificial Sequence Synthetic oligonucleotide
ggggtctttgctcagggcggaccaggtgctcaggtagtgg

SEQ ID NO: 206 - DNA, 40 bp, Artificial Sequence
  Description of Artificial Sequence Synthetic oligonucleotide
ggggtctttgctcagggcggatcgggtgctcaggtagtgg

SEQ ID NO: 207 - DNA, 40 bp, Artificial Sequence
  Description of Artificial Sequence Synthetic oligonucleotide
ggggtctttgctcagggcgggttgggtgctcaggtagtgg

SEQ ID NO: 208 - DNA, 40 bp, Artificial Sequence
  Description of Artificial Sequence Synthetic oligonucleotide
ggggtctttgctcagggcgagctgggtgctcaggtagtgg

SEQ ID NO: 209 - DNA, 40 bp, Artificial Sequence
  Description of Artificial Sequence Synthetic oligonucleotide
ggggtctttgctcagggcaaactgggtgctcaggtagtgg

SEQ ID NO: 210 - DNA, 40 bp, Artificial Sequence
  Description of Artificial Sequence Synthetic oligonucleotide
ggggtctttgctcagggtagactgggtgctcaggtagtgg

SEQ ID NO: 211 - DNA, 40 bp, Artificial Sequence
  Description of Artificial Sequence Synthetic oligonucleotide
ggggtctttgctcaggatggactgggtgctcaggtagtgg

SEQ ID NO: 212 - DNA, 40 bp, Artificial Sequence
  Description of Artificial Sequence Synthetic oligonucleotide
ggggtctttgctcagaacggactgggtgctcaggtagtgg

SEQ ID NO: 213 - DNA, 40 bp, Artificial Sequence
  Description of Artificial Sequence Synthetic oligonucleotide
ggggtctttgctcaaagcggactgggtgctcaggtagtgg

SEQ ID NO: 214 - DNA, 40 bp, Artificial Sequence
  Description of Artificial Sequence Synthetic oligonucleotide
ggggtctttgctcgaggcggactgggtgctcaggtagtgg

SEQ ID NO: 215 - DNA, 40 bp, Artificial Sequence
  Description of Artificial Sequence Synthetic oligonucleotide
ggggtctttgcttggggcggactgggtgctcaggtagtgg

SEQ ID NO: 216 - DNA, 40 bp, Artificial Sequence
  Description of Artificial Sequence Synthetic oligonucleotide
ggggtctttgcctagggcggactgggtgctcaggtagtgg

SEQ ID NO: 217 - DNA, 40 bp, Artificial Sequence
  Description of Artificial Sequence Synthetic oligonucleotide
ggggtctttgtccagggcggactgggtgctcaggtagtgg

SEQ ID NO: 218 - DNA, 40 bp, Artificial Sequence
  Description of Artificial Sequence Synthetic oligonucleotide
ggttactgccagttgaatctgagggtccaccaaacgtaat

SEQ ID NO: 219 - DNA, 40 bp, Artificial Sequence
  Description of Artificial Sequence Synthetic oligonucleotide
ggttactgccggttgaatctgagggtccaccaaacgtaat

SEQ ID NO: 220 - DNA, 40 bp, Artificial Sequence
  Description of Artificial Sequence Synthetic oligonucleotide
ggttactgccaattgaatctgagggtccaccaaacgtaat

SEQ ID NO: 221 - DNA, 40 bp, Artificial Sequence
  Description of Artificial Sequence Synthetic oligonucleotide
ggttactgccagctgaatctgagggtccaccaaacgtaat

SEQ ID NO: 222 - DNA, 40 bp, Artificial Sequence
  Description of Artificial Sequence Synthetic oligonucleotide
ggttactgccagtcgaatctgagggtccaccaaacgtaat

SEQ ID NO: 223 - DNA, 40 bp, Artificial Sequence
  Description of Artificial Sequence Synthetic oligonucleotide
ggttactgccagttaaatctgagggtccaccaaacgtaat

SEQ ID NO: 224 - DNA, 40 bp, Artificial Sequence
  Description of Artificial Sequence Synthetic oligonucleotide
ggttactgccagttggatctgagggtccaccaaacgtaat

SEQ ID NO: 225 - DNA, 40 bp, Artificial Sequence
  Description of Artificial Sequence Synthetic oligonucleotide
ggttactgccagttgagtctgagggtccaccaaacgtaat

SEQ ID NO: 226 - DNA, 40 bp, Artificial Sequence
  Description of Artificial Sequence Synthetic oligonucleotide
ggttactgccagttgaacctgagggtccaccaaacgtaat

SEQ ID NO: 227 - DNA, 40 bp, Artificial Sequence
  Description of Artificial Sequence Synthetic oligonucleotide
ggttactgccagttgaatttgagggtccaccaaacgtaat

SEQ ID NO: 228 - DNA, 40 bp, Artificial Sequence
  Description of Artificial Sequence Synthetic oligonucleotide
ggttactgccagttgaatccgagggtccaccaaacgtaat

SEQ ID NO: 229 - DNA, 40 bp, Artificial Sequence
  Description of Artificial Sequence Synthetic oligonucleotide
ggttactgccagttgaatctaagggtccaccaaacgtaat

SEQ ID NO: 230 - DNA, 40 bp, Artificial Sequence
  Description of Artificial Sequence Synthetic oligonucleotide
ggttactgccagttgaatctgggggtccaccaaacgtaat

SEQ ID NO: 231 - DNA, 40 bp, Artificial Sequence
  Description of Artificial Sequence Synthetic oligonucleotide
ggttactgccagttgaatctgaaggtccaccaaacgtaat

SEQ ID NO: 232 - DNA, 40 bp, Artificial Sequence
  Description of Artificial Sequence Synthetic oligonucleotide
ggttactgccagttgaatctgagagtccaccaaacgtaat

SEQ ID NO: 233 - DNA, 40 bp, Artificial Sequence
  Description of Artificial Sequence Synthetic oligonucleotide
ggttactgccagttgaatctgaggatccaccaaacgtaat

SEQ ID NO: 234 - DNA, 40 bp, Artificial Sequence
  Description of Artificial Sequence Synthetic oligonucleotide
ggttactgccagttgaatctgagggcccaccaaacgtaat

SEQ ID NO: 235 - DNA, 40 bp, Artificial Sequence
  Description of Artificial Sequence Synthetic oligonucleotide
ggttactgccagttgaatctgagggttcaccaaacgtaat

SEQ ID NO: 236 - DNA, 40 bp, Artificial Sequence
  Description of Artificial Sequence Synthetic oligonucleotide
ggttactgccagttgaatctgagggtctaccaaacgtaat

SEQ ID NO: 237 - DNA, 40 bp, Artificial Sequence
  Description of Artificial Sequence Synthetic oligonucleotide
ggttactgccagttgaatctgagggtccgccaaacgtaat

SEQ ID NO: 238 - DNA, 40 bp, Artificial Sequence
  Description of Artificial Sequence Synthetic oligonucleotide
ggttactgccagttgaatctgagggtccatcaaacgtaat

SEQ ID NO: 239 - DNA, 40 bp, Artificial Sequence
  Description of Artificial Sequence Synthetic oligonucleotide
attacgtttggtggaccctcagattcaactggcagtaacc

SEQ ID NO: 240 - DNA, 40 bp, Artificial Sequence
  Description of Artificial Sequence Synthetic oligonucleotide
attacgtttggtggaccctcagattcaaccggcagtaacc

SEQ ID NO: 241 - DNA, 40 bp, Artificial Sequence
  Description of Artificial Sequence Synthetic oligonucleotide
attacgtttggtggaccctcagattcaattggcagtaacc

SEQ ID NO: 242 - DNA, 40 bp, Artificial Sequence
  Description of Artificial Sequence Synthetic oligonucleotide
attacgtttggtggaccctcagattcagctggcagtaacc

SEQ ID NO: 243 - DNA, 40 bp, Artificial Sequence
  Description of Artificial Sequence Synthetic oligonucleotide
attacgtttggtggaccctcagattcgactggcagtaacc

SEQ ID NO: 244 - DNA, 40 bp, Artificial Sequence
  Description of Artificial Sequence Synthetic oligonucleotide
attacgtttggtggaccctcagatttaactggcagtaacc

SEQ ID NO: 245 - DNA, 40 bp, Artificial Sequence
  Description of Artificial Sequence Synthetic oligonucleotide
attacgtttggtggaccctcagatccaactggcagtaacc

SEQ ID NO: 246 - DNA, 40 bp, Artificial Sequence
  Description of Artificial Sequence Synthetic oligonucleotide
attacgtttggtggaccctcagactcaactggcagtaacc

SEQ ID NO: 247 - DNA, 40 bp, Artificial Sequence
  Description of Artificial Sequence Synthetic oligonucleotide
attacgtttggtggaccctcaggttcaactggcagtaacc

SEQ ID NO: 248 - DNA, 40 bp, Artificial Sequence
  Description of Artificial Sequence Synthetic oligonucleotide
attacgtttggtggaccctcaaattcaactggcagtaacc

SEQ ID NO: 249 - DNA, 40 bp, Artificial Sequence
  Description of Artificial Sequence Synthetic oligonucleotide
attacgtttggtggaccctcggattcaactggcagtaacc

SEQ ID NO: 250 - DNA, 40 bp, Artificial Sequence
  Description of Artificial Sequence Synthetic oligonucleotide
attacgtttggtggacccttagattcaactggcagtaacc

SEQ ID NO: 251 - DNA, 40 bp, Artificial Sequence
  Description of Artificial Sequence Synthetic oligonucleotide
attacgtttggtggacccccagattcaactggcagtaacc

SEQ ID NO: 252 - DNA, 40 bp, Artificial Sequence
  Description of Artificial Sequence Synthetic oligonucleotide
attacgtttggtggaccttcagattcaactggcagtaacc

SEQ ID NO: 253 - DNA, 40 bp, Artificial Sequence
  Description of Artificial Sequence Synthetic oligonucleotide
attacgtttggtggactctcagattcaactggcagtaacc

SEQ ID NO: 254 - DNA, 40 bp, Artificial Sequence
  Description of Artificial Sequence Synthetic oligonucleotide
attacgtttggtggatcctcagattcaactggcagtaacc

SEQ ID NO: 255 - DNA, 40 bp, Artificial Sequence
  Description of Artificial Sequence Synthetic oligonucleotide
attacgtttggtgggccctcagattcaactggcagtaacc

SEQ ID NO: 256 - DNA, 40 bp, Artificial Sequence
  Description of Artificial Sequence Synthetic oligonucleotide
attacgtttggtgaaccctcagattcaactggcagtaacc

SEQ ID NO: 257 - DNA, 40 bp, Artificial Sequence
  Description of Artificial Sequence Synthetic oligonucleotide
attacgtttggtagaccctcagattcaactggcagtaacc

SEQ ID NO: 258 - DNA, 40 bp, Artificial Sequence
  Description of Artificial Sequence Synthetic oligonucleotide
attacgtttggcggaccctcagattcaactggcagtaacc

SEQ ID NO: 259 - DNA, 40 bp, Artificial Sequence
  Description of Artificial Sequence Synthetic oligonucleotide
attacgtttgatggaccctcagattcaactggcagtaacc

SEQ ID NO: 260 - DNA, 40 bp, Artificial Sequence
  Description of Artificial Sequence Synthetic oligonucleotide
ggttactgccgattgaatctgagggtccaccaaacgtaat

SEQ ID NO: 261 - DNA, 40 bp, Artificial Sequence
  Description of Artificial Sequence Synthetic oligonucleotide
ggttactgccaactgaatctgagggtccaccaaacgtaat

SEQ ID NO: 262 - DNA, 40 bp, Artificial Sequence
  Description of Artificial Sequence Synthetic oligonucleotide
ggttactgccagccgaatctgagggtccaccaaacgtaat

SEQ ID NO: 263 - DNA, 40 bp, Artificial Sequence
  Description of Artificial Sequence Synthetic oligonucleotide
ggttactgccagtcaaatctgagggtccaccaaacgtaat

SEQ ID NO: 264 - DNA, 40 bp, Artificial Sequence
  Description of Artificial Sequence Synthetic oligonucleotide
ggttactgccagttagatctgagggtccaccaaacgtaat

SEQ ID NO: 265 - DNA, 40 bp, Artificial Sequence
  Description of Artificial Sequence Synthetic oligonucleotide
ggttactgccagttgggtctgagggtccaccaaacgtaat

SEQ ID NO: 266 - DNA, 40 bp, Artificial Sequence
  Description of Artificial Sequence Synthetic oligonucleotide
ggttactgccagttgagcctgagggtccaccaaacgtaat

SEQ ID NO: 267 - DNA, 40 bp, Artificial Sequence
  Description of Artificial Sequence Synthetic oligonucleotide
ggttactgccagttgaacttgagggtccaccaaacgtaat

SEQ ID NO: 268 - DNA, 40 bp, Artificial Sequence
  Description of Artificial Sequence Synthetic oligonucleotide
ggttactgccagttgaattcgagggtccaccaaacgtaat

SEQ ID NO: 269 - DNA, 40 bp, Artificial Sequence
  Description of Artificial Sequence Synthetic oligonucleotide
ggttactgccagttgaatccaagggtccaccaaacgtaat

SEQ ID NO: 270 - DNA, 40 bp, Artificial Sequence
  Description of Artificial Sequence Synthetic oligonucleotide
ggttactgccagttgaatctaggggtccaccaaacgtaat

SEQ ID NO: 271 - DNA, 40 bp, Artificial Sequence
  Description of Artificial Sequence Synthetic oligonucleotide
ggttactgccagttgaatctggaggtccaccaaacgtaat

SEQ ID NO: 272 - DNA, 40 bp, Artificial Sequence
  Description of Artificial Sequence Synthetic oligonucleotide
ggttactgccagttgaatctgaaagtccaccaaacgtaat

SEQ ID NO: 273 - DNA, 40 bp, Artificial Sequence
  Description of Artificial Sequence Synthetic oligonucleotide
ggttactgccagttgaatctgagaatccaccaaacgtaat

SEQ ID NO: 274 - DNA, 40 bp, Artificial Sequence
  Description of Artificial Sequence Synthetic oligonucleotide
ggttactgccagttgaatctgaggacccaccaaacgtaat

SEQ ID NO: 275 - DNA, 40 bp, Artificial Sequence
  Description of Artificial Sequence Synthetic oligonucleotide
ggttactgccagttgaatctgagggctcaccaaacgtaat

SEQ ID NO: 276 - DNA, 40 bp, Artificial Sequence
  Description of Artificial Sequence Synthetic oligonucleotide
ggttactgccagttgaatctgagggtttaccaaacgtaat

SEQ ID NO: 277 - DNA, 40 bp, Artificial Sequence
  Description of Artificial Sequence Synthetic oligonucleotide
ggttactgccagttgaatctgagggtctgccaaacgtaat

SEQ ID NO: 278 - DNA, 40 bp, Artificial Sequence
  Description of Artificial Sequence Synthetic oligonucleotide
ggttactgccagttgaatctgagggtccgtcaaacgtaat

SEQ ID NO: 279 - DNA, 40 bp, Artificial Sequence
  Description of Artificial Sequence Synthetic oligonucleotide
attacgtttggtggaccctcagattcaatcggcagtaacc

SEQ ID NO: 280 - DNA, 40 bp, Artificial Sequence
  Description of Artificial Sequence Synthetic oligonucleotide
attacgtttggtggaccctcagattcagttggcagtaacc

SEQ ID NO: 281 - DNA, 40 bp, Artificial Sequence
  Description of Artificial Sequence Synthetic oligonucleotide
attacgtttggtggaccctcagattcggctggcagtaacc

SEQ ID NO: 282 - DNA, 40 bp, Artificial Sequence
  Description of Artificial Sequence Synthetic oligonucleotide
attacgtttggtggaccctcagatttgactggcagtaacc

SEQ ID NO: 283 - DNA, 40 bp, Artificial Sequence
  Description of Artificial Sequence Synthetic oligonucleotide
attacgtttggtggaccctcagatctaactggcagtaacc

SEQ ID NO: 284 - DNA, 40 bp, Artificial Sequence
  Description of Artificial Sequence Synthetic oligonucleotide
attacgtttggtggaccctcagacccaactggcagtaacc

SEQ ID NO: 285 - DNA, 40 bp, Artificial Sequence
  Description of Artificial Sequence Synthetic oligonucleotide
attacgtttggtggaccctcaggctcaactggcagtaacc

SEQ ID NO: 286 - DNA, 40 bp, Artificial Sequence
  Description of Artificial Sequence Synthetic oligonucleotide
attacgtttggtggaccctcaagttcaactggcagtaacc

SEQ ID NO: 287 - DNA, 40 bp, Artificial Sequence
  Description of Artificial Sequence Synthetic oligonucleotide
attacgtttggtggaccctcgaattcaactggcagtaacc

SEQ ID NO: 288 - DNA, 40 bp, Artificial Sequence
  Description of Artificial Sequence Synthetic oligonucleotide
attacgtttggtggacccttggattcaactggcagtaacc

SEQ ID NO: 289 - DNA, 40 bp, Artificial Sequence
  Description of Artificial Sequence Synthetic oligonucleotide
attacgtttggtggacccctagattcaactggcagtaacc

SEQ ID NO: 290 - DNA, 40 bp, Artificial Sequence
  Description of Artificial Sequence Synthetic oligonucleotide
attacgtttggtggacctccagattcaactggcagtaacc

SEQ ID NO: 291 - DNA, 40 bp, Artificial Sequence
  Description of Artificial Sequence Synthetic oligonucleotide
attacgtttggtggactttcagattcaactggcagtaacc

SEQ ID NO: 292 - DNA, 40 bp, Artificial Sequence
  Description of Artificial Sequence Synthetic oligonucleotide
attacgtttggtggattctcagattcaactggcagtaacc

SEQ ID NO: 293 - DNA, 40 bp, Artificial Sequence
  Description of Artificial Sequence Synthetic oligonucleotide
attacgtttggtgggtcctcagattcaactggcagtaacc

SEQ ID NO: 294 - DNA, 40 bp, Artificial Sequence
  Description of Artificial Sequence Synthetic oligonucleotide
attacgtttggtgagccctcagattcaactggcagtaacc

SEQ ID NO: 295 - DNA, 40 bp, Artificial Sequence
  Description of Artificial Sequence Synthetic oligonucleotide
attacgtttggtaaaccctcagattcaactggcagtaacc

SEQ ID NO: 296 - DNA, 40 bp, Artificial Sequence
  Description of Artificial Sequence Synthetic oligonucleotide
attacgtttggcagaccctcagattcaactggcagtaacc

SEQ ID NO: 297 - DNA, 40 bp, Artificial Sequence
  Description of Artificial Sequence Synthetic oligonucleotide
attacgtttgacggaccctcagattcaactggcagtaacc

(19) United States
(12) Patent Application Publication
(10) Pub. No.: US 2022/0380835 A1
(43) Pub. Date: Dec. 1, 2022

(54) CRISPR COMPLEX-BASED DETECTION SYSTEM AND METHOD

(71) Applicant: University of Florida Research Foundation, Inc., Gainesville, FL (US)

(72) Inventors: Piyush K. Jain, Gainesville, FL (US); Long T. Nguyen, Gainesville, FL (US); Brianna M Smith, Gainesville, FL (US); Brianna Pawlyshyn, Gainesville, FL (US); Marco Downing, Gainesville, FL (US)

(21) Appl. No.: 17/775,206

(22) PCT Filed: Nov. 7, 2020
(86) PCT No.: PCT/US2020/059577

§ 371 Date: May 6, 2022

Related U.S. Application Data
(60) Provisional application No. 63/078,708, filed on Sep. 15, 2020.
(60) Provisional application No. 63/031,865, filed on May 29, 2020.
(60) Provisional application No. 63/010,382, filed on Apr. 15, 2020.
(60) Provisional application No. 63/001,056, filed on Mar. 27, 2020.
(60) Provisional application No. 62/988,679, filed on Mar. 12, 2020.
(60) Provisional application No. 62/952,762, filed on Dec. 23, 2019.
(60) Provisional application No. 62/934,217, filed on Nov. 12, 2019.
(60) Provisional application No. 62/932,823, filed on Nov. 8, 2019.

(51) Int. Cl. C12Q 1/6816; C12N 15/11; C12N 9/22

(52) U.S. Cl. CPC C12Q 1/6816; C12N 15/11; C12N 9/22; C12N 2310/20; C12N 2310/315

(57) ABSTRACT

The present disclosure provides systems that include a CRISPR-associated (Cas) enzyme with trans cleavage activity; a guide CRISPR RNA (crRNA) including a guide sequence and a polynucleotide extension sequence, wherein the guide sequence is configured to bind to a target polynucleotide, the polynucleotide extension sequence is linked to 3′-end of the guide sequence; and a probe including an oligonucleotide element labeled with a detectable label, wherein a detectable signal or a detectable molecule is generated when the probe is cleaved by the CRISPR-associated enzyme. The present disclosure also provides modified Cas complexes having a crRNA including a guide sequence and a polynucleotide extension sequence as well as modified CRISPR-Cas complexes having a guide crRNA including a guide sequence, an optional extension sequence, a linker sequence, and a complementary sequence, such that a portion of the crRNA sequence forms a toehold conformation at the 3′ end of the guide sequence.

### RELATED APPLICATION

This application claims the benefit of U.S. Provisional Application Ser. No. 62/932,823, filed Nov. 8, 2019; U.S. Provisional Application Ser. No. 62/934,217, filed Nov. 12, 2019; U.S. Provisional Application Ser. No. 62/952,762, filed Dec. 23, 2019; U.S. Provisional Application Ser. No. 62/988,679, filed Mar. 12, 2020; U.S. Provisional Application Ser. No. 63/001,056, filed Mar. 27, 2020; U.S. Provisional Application Ser. No. 63/010,382, filed Apr. 15, 2020; U.S. Provisional Application Ser. No. 63/031,865, filed May 29, 2020; and U.S. Provisional Application Ser. No. 63/078,708, filed Sep. 15, 2020. Each of these applications is incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 5, 2020, is named 222107-2590_SL.txt and is 73,708 bytes in size.

### FIELD

The present disclosure relates to CRISPR complex-based systems and methods.

### BACKGROUND

The breakthrough of CRISPR/Cas (clustered regularly interspaced short palindromic repeats/CRISPR-associated) systems has transformed the slow-progressing field of genome engineering with astronomical applications in biology, agriculture, biotechnology, diagnostics and treatment of genetic disorders. Originally derived from bacterial adaptive immune systems, the CRISPR/Cas technology works by introducing a Cas nuclease that acts like molecular scissors and a short crRNA that serves as a guide and binds with Cas and directs the crRNA/Cas complex to the target site to create double-stranded cuts in the DNA or sometimes a single-stranded cut in the RNA. This specific target recognition and cleavage are also referred to as ‘cis cleavage’. Some CRISPR/Cas systems also exhibit collateral non-specific cleavage or ‘trans-cleavage’ of single-stranded nucleic acid immediately after the specific target recognition or ‘cis-cleavage’ of the target.

CRISPR is currently classified into two classes based on whether they require multiple Cas effector proteins (Class 1) or a single Cas effector (Class 2). Class 2 systems are of high interest as they are based on single Cas effector proteins and are further divided into type II, V, and VI. The type V and VI CRISPR/Cas systems such as CRISPR/Cas12, CRISPR/Cas13, and CRISPR/Cas14 (abbreviated as CRISPR/Cas12-14) are emerging as powerful tools for nucleic acid detection in addition to the applications in gene and RNA editing. The CRISPR/Cas12-14 systems exhibit collateral non-specific cleavage or ‘trans-cleavage’ of single-stranded nucleic acids after the specific target recognition or ‘cis-cleavage’ of the target. Both type V and VI systems are of special interest as they are based on single Cas effector proteins and can cleave dsDNA and ssRNA, some without requiring a longer tracrRNA, and they possess a trans-cleavage activity that can be applied for diagnostics. Using single-stranded nucleic acid-based FRET reporters, the trans-cleavage activity of type V and VI systems has been applied for the detection of target dsDNA and ssRNA targets at low nM (1-10 nM) concentrations without further amplification. However, no trans-cleavage signal could be observed below 10 nM of dsDNA without any DNA amplification using the Acidaminococcus sp. derived AsCas12a or below 1 nM (100 fmols) using Lachnospiraceae bacterium derived LbCas12a. However, by coupling these detection systems with isothermal amplification, low aM concentration of cell-free tumor DNA in lung cancer and viral RNA in human saliva and blood samples have been achieved.

When the CRISPR/Cas12-14 systems are combined with a FRET-based reporter, a fluorophore connected to a quencher via a short oligonucleotide sequence, the presence of the target activator can be confirmed. This phenomenon has been efficiently harnessed by SHERLOCK (Specific High-sensitivity Enzymatic Reporter unLOCKing) and DETECTR (DNA Endonuclease Targeted CRISPR Trans Reporter) to reliably detect nucleic acids. It would be useful to improve the sensitivity and specificity of the CRISPR/Cas system.

### SUMMARY

In one aspect, the present disclosure provides a nucleic acid detection system including a CRISPR-associated (Cas) enzyme with trans cleavage activity; a guide CRISPR RNA (crRNA) comprising a guide sequence and a polynucleotide extension sequence, wherein the guide sequence is configured to bind to a target polynucleotide, the polynucleotide extension sequence is linked to a 3′-end of the guide sequence, and the polynucleotide extension sequence includes a ssDNA or ssRNA having 1-19 nucleotides; and a plurality of probes, each probe including an oligonucleotide element labeled with a detectable label, wherein the probe is configured to be cleaved by the Cas enzyme when the guide sequence binds the target polynucleotide to generate a detectable signal or a detectable molecule.

In another aspect, the present disclosure provides a modified guide CRISPR RNA (crRNA) adapted to form a ribonucleoprotein complex with a CRISPR-associated Cas12 enzyme, wherein the guide crRNA comprises a guide CRISPR RNA (crRNA) including a guide sequence and a polynucleotide extension sequence, wherein the guide sequence is configured to bind to a target polynucleotide, the polynucleotide extension sequence is linked to 3′-end of the guide sequence, and the polynucleotide extension sequence comprises a ssDNA or ssRNA having 1-19 nucleotides.

In another aspect, the present disclosure provides a method of detecting a target polynucleotide in a sample obtained from a subject. The method includes the step of contacting the sample with the detection system disclosed herein, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and detecting the signal or the molecule, wherein detection of the signal or the molecule indicates presence of the target polynucleotide in the sample.

In another aspect, the present disclosure provides a method of detecting a target polynucleotide in a sample obtained from a subject. The method includes the steps of contacting a sample from the subject with a detection system, wherein the detection system includes a CRISPR-associated (Cas) enzyme with trans cleavage activity, a guide CRISPR RNA (crRNA) comprising a guide sequence, and a probe including an oligonucleotide element labeled with a detectable label, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and detecting the signal or the molecule, wherein detection of the signal or the molecule indicates presence of the target polynucleotide in the sample; wherein the sample and the detection system are incubated at a temperature of 40-60° C. for a period of time.

Other systems, methods, features, and advantages of the present disclosure will be or will become apparent to one with skill in the art upon examination of the following drawings and detailed description. It is intended that all such additional systems, methods, features, and advantages be included within this description, and be within the scope of the present disclosure.

### BRIEF DESCRIPTION OF THE DRAWINGS

Further aspects of the present disclosure will be more readily appreciated upon review of the detailed description of its various embodiments, described below, when taken in conjunction with the accompanying drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-1H show trans-cleavage activity of a system of the present disclosure with Cas12a and modified crRNA via fluorescence-quencher-based reporter assay with TA rich fluorophore-quencher systems tested. (FIG. 1A) Schematic diagram comparing a system of the present disclosure using Cas12a with modified crRNAs to Cas12a with wild type crRNA with detection probes. The modified crRNA is extended on either the 3′- or 5′-ends with ssDNA, ssRNA, or phosphorothioate ssDNA. (FIG. 1B) A representation of a fluorescence-quencher-based trans-cleavage reporter assay image taken by GE Amersham Typhoon. (FIG. 1C), (FIG. 1D), and (FIG. 1E) 3′-end ssDNA, ssRNA and phosphorothioate ssDNA extensions of crRNA, respectively. (FIG. 1F), (FIG. 1G), and (FIG. 1H) 5′-end ssDNA, ssRNA and phosphorothioate ssDNA extensions of crRNA, respectively. The fold in fluorescence was normalized by taking the ratio of background-corrected fluorescence signals of sample with activator to the corresponding sample without activator. For FIGS. 1C-1H, error bars represent mean±SEM, where n=6 replicates (three technical replicates examined over two independent experiments). Statistical analysis was performed using two-way ANOVA test with Dunnett's multiple comparison test, where ns=not significant with p>0.05, and the asterisks (*, **, ***, ****) denote significant differences with p values listed above.

FIGS. 2A-2G show effect of reporter sequences on trans-cleavage activity, proposed mechanism of crRNA end processing, enzyme kinetics, and binding affinity of LbCas12a with modified crGFP. (FIG. 2A) Effect of different types of fluorophore-quencher systems on trans-cleavage activity with various modifications of crRNA. (FIG. 2B) Interactions of fluorescently labeled crRNAs with LbCas12a and dsDNA activator, characterized by PAGE analysis. In the absence of the activator, the modified crRNA (pre-crRNA) is trimmed by LbCas12a on its 5′-end (the first Uracil is cleaved, so-called truncated-crRNA or tru-crRNA). In the presence of the activator, the crRNA extensions are further trimmed, possibly leaving a 3′ overhang. (FIG. 2C) Schematic diagram of putative processing of crRNA cleavage sites in the presence and absence of activator GFP. (FIG. 2D) Enzyme kinetic data of LbCas12a with crGFP vs. crGFP+3′DNA7. (FIG. 2E) Michaelis-Menten kinetic study of the wild-type crGFP vs. crGFP+3′DNA7. For this assay, 100 nM of LbCas12a, 100 nM of crRNA, and 7.4 nM of GFP fragment were used. Error bars represent mean±SD, where n=3 technical replicates. (FIG. 2F) Time-dependent cis-cleavage of LbCas12a on GFP in the presence of nonspecific ssDNA M13mp18. The reaction mixture was taken out every five minutes and quenched with 6×SDS-containing loading dye. (FIG. 2G) Dissociation constants of crGFP vs. crGFP+3′DNA7. The K_{d} was determined by the biolayer interferometry binding kinetic assay with R²>0.9. Error bars represent ±SD, where n=5 independent dilutions.

FIGS. 3A-3E show characterization of a system of the present disclosure (also referred to herein and in the Examples as “ENHANCE” or “CRISPR ENHANCE”) with various crRNA modifications and different Cas12a systems. (FIG. 3A) Comparison of trans-cleavage activity between precursor crRNA (pre-crRNA) and mature crRNA (tru-crRNA, where the first Uracil on the 5′-end of the crRNA is cleaved by LbCas12a in the absence of the activator). (FIG. 3B) Comparison of trans-cleavage activity between AT-rich extensions and GC-rich 7-nt DNA 3′-end extensions on the crRNA+3′DNA7. For (a) and (b), error bars denote mean±SD, where n=3 technical replicates. (FIG. 3C) Trans-cleavage activity of LbCas12a with non-fully phosphorothioate (PS) modified crRNA targeting GFP fragment. Sequence representation of 6 non-fully PS extension on the 3′-end of crGFP ranging from 1 to 6 PS. The asterisk symbol (*) signifies the phosphorothioated nucleotide. The graph below the sequence representation shows fold change of the LbCas12a fluorescence-based reporter assay with the activator normalized to the corresponding samples without the activator at t=20 minutes. (FIG. 3D) kinetics of the LbCas12a fluorescence-based reporter assay in (FIG. 3C). FIG. 3E) Trans-cleavage activity of different variants of Cas12a. The prefix Lb, As, and Fn stand for Lachnospiraceae bacterium, Acidaminococcus, and Francisella novicida, respectively. For (FIG. 3C), (FIG. 3D), and (FIG. 3E), n=6 replicates (three technical replicates examined over two independent experiments), where error bars in (e) represent mean±SEM.

FIGS. 4A-4D show improved specificity of LbCas12a trans-cleavage with ENHANCE. (FIG. 4A) Single-point mutations (S1-S20) and (FIG. 4B) double-point mutations (D1-D19) on the target strand of a dsDNA GFP activator. (FIG. 4A) Superimposed bar graphs indicating fold change in fluorescence of the S 1-S20 mutant GFP activators normalized to the corresponding wild-type activator, and each of the S1-S20 mutant GFP activators has a single-point mutation. (FIG. 4B) Superimposed bar graphs indicating fold change in fluorescence of the D1-D19 mutant GFP activators normalized to the corresponding wild-type activator, each of the D1-D19 mutant GFP activators has a double-point mutation. (FIG. 4C) Single-point mutations (S1-S20) and (FIG. 4D) double-point mutations (D1-D19) on the target strand of a dsDNA SARS-CoV-2 activator. (FIG. 4C) Superimposed bar graphs indicating fold change in fluorescence of the S 1-S20 mutant SARS-CoV-2 activators normalized to the corresponding wild-type activator, and each of the S1-S20 mutant SARS-CoV-2 activators has a single-point mutation. (FIG. 4D) Superimposed bar graphs indicating fold change in fluorescence of the D1-D19 mutant SARS-CoV-2 activators normalized to the corresponding wild-type activator, each of the D1-D19 SARS-CoV-2 activator has a double-point mutation. All the values (FIG. 4A-4D) were plotted after 20 minutes of incubation of various activators with wild-type CRISPR or ENHANCE. For (FIG. 4A), n=6 (three technical replicates examined over two independent experiments). For FIGS. 4B-4D, n=4 (two technical replicates examined over two independent experiments). For FIGS. 4A-4D, values indicate mean±SEM and the statistical analysis was performed using two-way ANOVA test with Dunnett's multiple comparison test and only significant (p<0.05) values were marked with an asterisk (*) indicated as follows: *p<0.05, **p<0.01, ***p<0.001, and ****P<0.0001. A fold change in specificity was calculated and reported for only statistically significant mutants by taking the ratio of the normalized data for crRNA-WT to crRNA-3′DNA7.

FIGS. 5A-5I show improved detection of various targets using modified crRNA/LbCas12a system. (FIG. 5A) Effect of Magnesium ion on the trans-cleavage activity. Error bars represent mean±SD, where n=3 technical replicates. Statistical analysis was performed using two-way ANOVA test with Sidak's multiple comparison test, where ns=not significant, and the asterisk (*) denotes p values. (FIG. 5B) Limit of detection in femtomolar targeting PCA3 in simulated human urine at optimized Mg²⁺ concentration. (FIG. 5C) Raw fluorescence data showing detectable signal difference at 1 pM of PCA3 DNA. (FIG. 5D) Fold change in fluorescence signal of the modified crRNA+3′DNA7 targeting GFP after the recombinase polymerase amplification (RPA) step. (FIG. 5E) Effect of heteroduplex DNA-RNA and methylated activators on the trans-cleavage activity of LbCas12a. (FIG. 5F) trans-cleavage activity of different DNA targets. GFP, PCA3, COVID-19, and HCV are dsDNA activators, and their fluorescence shown were taken after 3 hours. The HIV target is ssDNA or cDNA/RNA heteroduplex, and its fluorescence signal shown was taken after 1 hour. (FIG. 5G) Limit of detection targeting HIV cDNA fragment after 15 and 30 minutes. Results in (FIG. 5B) and (FIG. 5G) are based on limit of detection calculations. (FIG. 5H) Fold change in trans-cleavage activity with LbCas12a in presence of 100 pM (10 fmols) of target HCV ssDNA. Using modified crRNA, the limit of detection of HCV target ssDNA was found to be 290 fM (29 amoles) at 30 min, without target amplification. (i) Fold change in trans-cleavage activity with LbCas12a in presence of 100 pM (10 fmols) of target SARS-CoV-2 cDNA (dsDNA). For (FIG. 5H), error bars represent mean±SEM, where n=9 replicates (three technical replicates examined over three independent experiments). For (FIG. 5E), (FIG. 5F), and (FIG. 5I), error bars represent mean±SEM, where n=6 replicates (three technical replicates examined over two independent experiments). For (FIG. 5E), (FIG. 5F), and (FIG. 5I), statistical analysis was performed using two-way ANOVA test with Dunnett's multiple comparison test, where ns=not significant with p>0.05, and the asterisks (*, **, ***, ****) denote significant differences with p values listed above.

FIGS. 6A-6E show improved detection of COVID-19 in a lateral flow assay using ENHANCE. (FIG. 6A) Schematic diagram showing how a lateral flow assay works. Briefly, the dipstick uses gold-labeled FITC-specific antibodies that bind to FITC-biotin reporter and travel through membrane. Only cleaved reporter will reside at the positive line. (FIG. 6B) Lateral flow assay detecting SARS-CoV-2 RNA N gene using crCov-2 and crCoV-2+3′DNA7 with RT-LAMP, and (FIG. 6C) band-intensity analysis of (FIG. 5B) using ImageJ. (FIG. 6D) Lateral flow assay detecting SARS-CoV-2 cDNA using crCov-2 and crCoV-2+3′DNA7 without a pre-amplification step, “T” indicates test (with SARS-CoV-2 RNA) and “C” indicates control (No SARS-CoV-2 RNA). (FIG. 6E) band-intensity analysis of (FIG. 6D) using ImageJ.

FIGS. 7A-7E show enhanced sensitivity and specificity with ENHANCE for detecting SARS-CoV-2 genomic RNA. (FIG. 7A) crRNA specificity towards SARS-CoV-2 and other highly similar pathogens from the same family. The targets were dsDNA amplified from plasmid controls 2019-nCoV_N_Positive Control, MERS-CoV Control, and SARS-CoV Control (IDT). (FIG. 7B) Detection reaction in (FIG. 7A) scanned by Typhoon (Amersham, GE healthcare). (FIG. 7C) crRNA specificity towards genomic RNA of SARS-CoV-2 and other genomic RNAs of highly similar pathogens from the same family. The targets were genomic RNA obtained from BEI Resources. (FIG. 7D) Lateral flow assay of (FIG. 7C). (FIG. 7E) Detection reaction in (c) scanned by Typhoon (Amersham, GE healthcare). For (FIG. 7A), and (FIG. 7C), error bars represent mean±SEM, where n=6 replicates (three technical replicates examined over two independent experiments).

FIGS. 8A-8C show trans-cleavage activity of LbCas12a with modified 3′ crRNA via fluorescence-quencher reporter assay with FAM-GC, where FAM-GC is a reporter shown above. F stands for fluorophore, and Q stands for quencher. (FIG. 8A) DNA, (FIG. 8B) RNA, and (FIG. 8C) PSDNA extensions of crRNA. The fold in fluorescence was normalized by taking the ratio of background-corrected fluorescence signals of the samples with the activator to the corresponding samples without the activator. Error bars in FIGS. 8A-8C represent mean±SEM, where n=6 replicates (three technical replicates examined over two independent experiments). Statistical analysis was performed using two-way ANOVA test with Dunnett's multiple comparison test, where ns=not significant, and the asterisk (*) denotes p values.

FIGS. 9A-9C show trans-cleavage activity of LbCas12a with modified 3′ crRNA via fluorescence-quencher reporter assay with HEX-TA, where HEX-TA is a reporter shown above. F stands for fluorophore, and Q stands for quencher. (FIG. 9A) DNA, (FIG. 9B) RNA, and (FIG. 9C) PSDNA extensions of crRNA. The fold in fluorescence was normalized by taking the ratio of background-corrected fluorescence signals of the samples with the activator to the corresponding samples without the activator. Error bars in FIGS. 9A-9C represent mean±SEM, where n=6 replicates (three technical replicates examined over two independent experiments). Statistical analysis was performed using two-way ANOVA test with Dunnett's multiple comparison test, where ns=not significant, and the asterisk (*) denotes p values.

FIGS. 10A-10C show trans-cleavage activity of LbCas12a with modified 5′ crRNA via fluorescence-quencher reporter assay with FAM-GC, where FAM-GC is a reporter shown above. F stands for fluorophore, and Q stands for quencher. (FIG. 10A) DNA, (FIG. 10B) RNA, and (FIG. 10C) PSDNA extensions of crRNA. The fold in fluorescence was normalized by taking the ratio of background-corrected fluorescence signals of the samples with the activator to the corresponding samples without the activator. Error bars in FIG. 10A-10C represent mean±SEM, where n=6 replicates (three technical replicates examined over two independent experiments). Statistical analysis was performed using two-way ANOVA test with Dunnett's multiple comparison test, where ns=not significant, and the asterisk (*) denotes p values.

FIGS. 11A-11C Trans-cleavage activity of LbCas12a with modified 5′ crRNA via fluorescence-quencher reporter assay with HEX-TA, where HEX-TA is a reporter shown above. F stands for fluorophore, and Q stands for quencher. (FIG. 11A) DNA, (FIG. 11B) RNA, and (FIG. 11C) PSDNA extensions of crRNA. The fold in fluorescence was normalized by taking the ratio of background-corrected fluorescence signals of the samples with the activator to the corresponding samples without the activator. Error bars in FIGS. 11A-11C represent mean±SEM, where n=6 replicates (three technical replicates examined over two independent experiments). Statistical analysis was performed using two-way ANOVA test with Dunnett's multiple comparison test, where ns=not significant, and the asterisk (*) denotes p values.

FIGS. 12A-12C show representatives of samples from the fluorescence-quencher reporter assay. (FIG. 12A) Fluorescence measurement in relative fluorescence unit (RFU) of LbCas12a reaction with 3′-end modified crGFP. (FIG. 12B) Fluorescence reading of the Michaelis-Menten study. The substrate used in this experiment was /5HEX/TTATT/3IABkFQ/. (FIG. 12C) Fluorescence image of (FIG. 12B) scanned by Amersham Typhoon.

FIG. 13A-13B show cis-cleavage of LbCas12a with fluorescently-labeled extended crGFP. (FIG. 13A) The 3′-end extensions of crGFP were varied from 0 to 19-mer DNA and tagged with FAM/HEX. (FIG. 13B) The proposed mechanism for LbCas12a processing of modified crGFP as observed from (FIG. 13A). The experiment in (FIG. 13A) was repeated three times with similar results.

FIGS. 14A-14B show gel images of cis-cleavage assay of LbCas12a with different crRNAs carrying a fluorophore-quencher pair on either (FIG. 14A) 3′ end or (FIG. 14B) 5′ end. crCon (scrambled crRNA), crGFP (GFP targeting crRNA), (FIG. 14A) crGFP+3′ DNA13, and crGFP+3′ DNA7+Cy5+DNA6+Iowa Black RQ or (FIG. 14B) 5′-end modified crRNAs including crGFP+5′ DNA19 and crGFP+5′ DNA13+Cy5+DNA6+Iowa Black RQ. Cy5 is indicated in red and DNA stained with GelRed is shown in blue. The 5′ end modified crRNAs showed cleavage of crRNA immediately after adding the LbCas12a but before adding the activator. However, both 3′ and 5′ end modified crRNAs, showed increase in signal intensity after activator addition indicating trans cleavage of the crRNA. 250 nM of LbCas12a, 250 nM of crRNA, and 7.4 nM of DNA activator fragment were used. The experiments in (FIG. 14A) and (FIG. 14B) were repeated twice with similar results.

FIG. 15 show a representation of the biolayer interferometry (BLI) binding kinetics. The picture shown above is the binding kinetic study of LbCas12a to crGFP+3′DNA7. The experiment was carried in five steps: baseline1, loading, baseline2, association, and dissociation (see materials and methods). The y-axis represents response of LbCas12a and crRNA to the biosensor in nm. Data were trimmed and processed using the Octet Data Analysis 10.0 software, and only K_{D} values with R²>0.9 were selected.

FIGS. 16A-16B show trans-cleavage activity of LbCas12a with modified crGFP+3′DNA7 with either GC rich or TA rich region via fluorescence-quencher reporter assay at varying Mg²⁺ concentration. F stands for fluorophore, and Q stands for quencher. (FIG. 16A) FAM-TA was used, (FIG. 16B) FAM-GC was used. The fold in fluorescence was normalized by taking the ratio of background-corrected fluorescence signals of the samples with the activator to the corresponding samples without the activator. Error bars represent mean±SD, where n=3 technical replicates.

FIG. 17 shows typhoon image (scanned with Amersham Typhoon, GE Healthcare) of the LbCas12a fluorescence-based reporter assay in FIG. 2C,D above.

FIG. 18 show cis-cleavage and trans-cleavage activity of LbCas12 with modified crGFP and crGFP+3′DNA7 with LbCas12a, AsCas12a, and FnCas12a. The cis-cleavage reaction was loaded on 1% native agarose gel. The experiment was repeated twice with similar results.

FIGS. 19A-19B show cis-cleavage of LbCas12a with 3′-end modified crGFP. For this assay, 100 nM of LbCas12a, 100 nM of crRNA, and 7.4 nM of GFP fragment were used. crCon represents nonspecific crRNA. (FIG. 19A) 1% agarose gel image. (FIG. 19B) Percent cleavage of the GFP fragment calculated in (a), where error bars represent mean±SD with n=2 replicates. The experiment was repeated more than twice with similar results.

FIGS. 20A-20B show cis-cleavage of LbCas12a with 5′-end modified crGFP. For this assay, 100 nM of LbCas12a, 100 nM of crRNA, and 7.4 nM of GFP fragment were used. crCon represents nonspecific crRNA. (FIG. 20A) 1% agarose gel image. (FIG. 20B) Percent cleavage of the GFP fragment calculated in (a), where error bars represent mean±SD with n=2 replicates. The experiment was repeated more than twice with similar results.

FIG. 21A shows the effect of a divalent ion on the cis-cleavage assay of LbCas12a with different crRNAs including wild-type crGFP (top-left quadrant), crGFP+3′DNA7 (top-right quadrant), crGFP+3′RNA7 (bottom-left quadrant), and crGFP+3′PSDNA7 (bottom-right quadrant). Neg Ctrl represents the negative control where crCon was used in the reaction mixture. Pos Ctrl represents the positive control where NEBuffer 2.1 was added. In this experiment, 100 nM of LbCas12a, 100 nM of crRNA, and 6.6 nM of DNA activator fragment were used. FIG. 21B shows percent cleavage analyzed in (a). Each metal ion was added to LbCas12a reaction to a final concentration of 3 mM in cleavage buffer (50 mM NaCl, 10 mM Tris-HCl, 10 mM MgCl₂, 100 μg/ml BSA, pH 7.9). The graph indicated mean from a pilot experiment in the graph (n=1) that was not repeated. Only Mg²⁺ and Zn²⁺ were further studied (repeated twice with similar results).

FIGS. 22A-22C show the effect of Zn²⁺ on Cis-cleavage assay of LbCas12a with different crRNAs in the presence of Mg²⁺. 200 nM of LbCas12a, 200 nM of crRNA, 6.6 nM of DNA activator fragment, and 3 mM of Mg²⁺ were used. # stands for non-target dsDNA fragment. (FIG. 22A) crGFP and crGFP+3′DNA7. (FIG. 22B) crGFP+3′DNA13 and crGFP+3′DNA19. (FIG. 22C) Percent cleavage in (FIG. 22A) and (FIG. 22B). Zn²⁺ ion concentration was serial diluted and added to the LbCas12a reaction in the presence cleavage buffer (NEBuffer 2.1 with 3 mM Mg²⁺ final concentration. The experiments in (FIG. 22A) and (FIG. 22B) were repeated once.

FIG. 23 shows time-dependent cis-cleavage of LbCas12a on GFP in the presence of nonspecific ssDNA M13mp18 at varying Mg²⁺ concentration. The reaction mixture was taken out every five minutes and quenched with 6×SDS-containing loading dye.

FIG. 24 shows Michaelis-Menten kinetic study of the wild type crPCA3 vs. crPCA3+3′DNA7. The graph shows initial velocity as a function of substrate concentration. In this case, the substrate used was /5HEX/TTATT/3IABkFQ/.

FIG. 25 shows trans-cleavage activity of LbCas12 with modified 3′ crPCA3 via fluorescence-quencher reporter assay FAM-TA at varying Mg²⁺ concentration. FAM-TA is a reporter shown above. F stands for fluorophore, and Q stands for quencher. The fold in fluorescence was normalized by taking the ratio of background-corrected fluorescence signals of the samples with the activator to the corresponding samples without the activator. Error bars represent mean±SEM, where n=6 replicates (three technical replicates examined over two independent experiments).

FIG. 26 shows limit of detection using modified crRNA/LbCas12a system. Limit of detection in femtomolar at 13 mM Mg²⁺ concentration. The reaction was carried out by adding simulated human urine spiked with either dsDNA GFP or PCA3 fragments to the LbCas12a reaction mixture.

FIGS. 27A-27B show LbCas12a trans-cleavage with different modified crRNAs and double-stranded activators (target and non-target strands annealed in the ratio of 1:1) measured by a fluorescence-quencher reporter assay (FAM-TA) in triplicates (n=3). The data shows results at 81 minutes for (FIG. 27A) dsDNA, dsMeC, sRNA, and (FIG. 27B) dsDNA, DNA/RNA, RNA/DNA. The values were normalized to their respective crRNA without activators. The error bars in (FIG. 27A) and (FIG. 27B) denote mean±SD, where n=3 technical replicates. Statistical analysis was performed using two-way ANOVA test with Dunnett's multiple comparison test, where the asterisk (*) denotes p values.

FIG. 28A shows trans-cleavage activity of regular crRNA vs. 3′DNA7 modified crRNA for detecting HIV ssDNA with LbCas12a over 30 minutes in the presence of various concentrations of HIV target dsDNA. Blank subtracted raw fluorescence intensities are indicated. Error bars represents mean±SD, where n=3 technical replicates. FIG. 28B shows fold change in fluorescence intensity in the presence vs. absence of target is indicated for various HIV ssDNA vs. dsDNA activators, where ssT indicates ssDNA target strand, ssNT indicates ssDNA non-target strand and ds indicates double-stranded DNA target. Error bars represent mean±SEM where n=6 replicates (three technical replicates examined over two independent experiments). (FIG. 28C) Trans-cleavage of ssHIV using engineered CRISPR/LbCas12a. Comparison of single-stranded (ss) vs. double-stranded DNA (ds) targets analysis after 30 minutes is shown in bar graphs. HIV-1 ssDNA from Tat gene (IDT Technologies). The modified crRNA showed much higher sensitivity than he regular crRNA with fM detection limits within 30 minutes. For ssHIV-T, ssHIV-NT, and dsHIV, error bars represent mean±SEM with n=6 replicates (three technical replicates examined over two independent experiments). For HIV RNA, error bars represent mean with n=2 replicates.

FIG. 29A-29B show trans-cleavage activity of LbCas12a over time in the presence of 10 nM (100 pmols) of HCV non-target ssDNA (FIG. 29A) and HCV dsDNA (FIG. 29B). Using engineered crRNA with optimized CRISPR assay, detection of HCV target ssDNA was found to be 29 amols (290 fM, 100 uL) at 30 min, without target amplification. Error bars represent mean±SEM, where n=9 (three technical replicates examined over three independent experiments).

FIG. 30 shows trans-cleavage activity of LbCas12a over time in the presence of 10 nM (100 pmols) of SARS-CoV-2 dsDNA target (bottom). Using engineered crRNA, containing 3′DNA7, and optimized CRISPR assay, the detection of SARS-CoV-2 target ssDNA was found to be significantly faster compared to the wild type crRNA, without target amplification. Error bars represents mean±SEM, where n=6 (three technical replicates examined over two independent experiments).

FIGS. 31A-31B show determination of limit of detection for SARS-CoV-2 RNA using LbCas12a. To detect RNA, first a reverse transcriptase step was formed to convert RNA into DNA/RNA heteroduplex. The heteroduplex was detected by using an optimized CRISPR assay over time using either wild type crRNA (crCoV-WT) or engineered crRNA containing 3′DNA7 modifications (crCoV+3′DNA7). The limit of detection for SARS-CoV-2 target RNA was found to be significantly lower with crCoV+3′DNA7, compared to the wild type crRNA, without target amplification. (FIG. 31A) LoD were plotted from two independent experiments. (FIG. 31B) is representative data with the lowest limit of detection from (FIG. 31A).

FIGS. 32A-32B show determination of lowest limit of detection for SARS-CoV-2 dsDNA using LbCas12a with an optimized CRISPR assay over time using either wild type crRNA (crCoV-WT) or engineered crRNA containing 3′DNA7 modifications (crCoV+3′DNA7). The limit of detection for SARS-CoV-2 target RNA was found to be significantly lower with crCoV+3′DNA7 (130 fM), compared to the crCoV-WT (750 fM) within 45 minutes, without target amplification. (FIG. 32A) LoD were plotted from two independent experiments. (FIG. 32B) is representative data with the lowest limit of detection from (a).

FIG. 33 shows band intensity analysis of paper-strip test of LbCas12a targeting SARS-CoV-2. In this experiment, incubation time and temperature were varied (see materials and methods for more details). The paper-strips were scanned under Typhoon Amersham (GE healthcare) and analyzed using ImageJ.

FIGS. 34A-34D show trans-cleavage activity of LbCas12a targeting SARS-CoV-2 with a RT-LAMP preamplification step via fluorescence-quencher reporter assay with FAM-TA. The data shown above are raw fluorescence signal measured by the fluorescence microplate reader Biotek Synergy 2. (FIG. 34A) and (FIG. 34B) are kinetics data in 15 min of crCoV-2-WT and crCoV-2+3′DNA7. In this experiment, the concentration of FQ reporter was doubled compared to previous experiments (100 nM). (FIG. 34C) is a single-point fluorescence signal extracted from (FIG. 34A) and (FIG. 34B). (FIG. 34D) Lateral flow assay with RT-LAMP preamplification step. The paper strips were scanned and analyzed using imageJ. Error bars represent in FIG. 34A-34C represent mean±SEM, where n=6 (three technical replicates examined over two independent experiments).

FIG. 35 shows a representation of time lapse pictures of the lateral flow assay targeting SARS-CoV-2 RNA N gene. The paper strips were immediately dipped into the LbCas12a reaction after the incubation. The testing concentration was 1 nM SARS-CoV-2 RNA.

FIGS. 36A-B show improved CRISPR sensitivity with improved specificity with modified crRNAs with stem loop (also referred to herein as “toehold”) structures. The scale on the right side of the heat map indicates average fluorescence intensity (n=3), where higher value indicates greater trans-cleavage activity of the reporter. The structure of crRNA (top) with circled data (bottom) demonstrated 257% higher sensitivity for on-target DNA (GFP) while maintaining 21% higher specificity against off-target DNA compared to wild-type crRNA. The on-target data represents the trans-cleavage activity with a perfect-match wild-type activator (gray) while the off-target data represents an average trans-cleavage activity calculated from 20 different single point-mutant activators tested individually.

FIG. 37 shows schema of CRISPR-Mediated Detection utilizing Cas12 highlighting the effect of temperature on fluorescent signal.

FIG. 38 shows comparison of trans-cleavage activity at various temperatures to 37° C. Concentrations are as follows: 500 nM FQ-Reporter, 30 nM LbCas12a, 30 nM crGFP+DNA7-3′, varied dsGFP-ACT, 1×NEB 2.1. Cas, crRNA and FQ-reporter were mixed and allowed to incubate for 5 min at desired temperature. Then Activator was added to the solution and they were further incubated for 15 min. They were then removed from the thermocyclers and pipetted into a 96 well black flat bottom plate and read with a Biotek Fluorescence plate reader. Replicates were performed in triplicates (n=3). Error bars represent standard deviation.

FIG. 39 shows temperature sensitivity study of CRISPR-Cas trans-cleavage at various incubation temperatures. Concentrations are as follows: 500 nM FQ-Reporter, 30 nM LbCas12a, 30 nM crGFP+DNA7-3′, 100 pm dsGFP-ACT, 1×NEB 2.1. Cas, crRNA and FQ-reporter were mixed and allowed to incubate for 5 min at desired temperature. Then Activator was added to the solution and they were further incubated for 15 min. They were then removed from the thermocyclers and pipetted into a 96 well black flat bottom plate and read with a Biotek Fluorescence plate reader. Replicates were performed in triplicates (n=3). Error bars represent standard deviation.

FIG. 40 shows temperature sensitivity study of CRISPR-Cas trans-cleavage at various incubation temperatures. Concentrations are as follows: 500 nM FQ-Reporter, 30 nM LbCas12a, 30 nM crGFP+DNA7-3′, 100 pm dsGFP-ACT, 1×NEB 2.1. Cas, crRNA and FQ-reporter were mixed and allowed to incubate for 5 min at desired temperature. Then Activator was added to the solution and they were further incubated for 15 min. They were then removed from the thermocyclers and pipetted into a 96 well black flat bottom plate and read with a Biotek Fluorescence plate reader. Replicates were performed in triplicates (n=3). Error bars represent standard deviation.

FIG. 41 shows trans-cleavage assay at 100 pM Activator for other Cas12 variants FnCas12a and AsCas12a. Error bars represent standard deviation with n=3.

### DETAILED DESCRIPTION

### Definitions

For convenience, before further description of the present invention, certain terms used in the specification, examples and appended claims are collected here. It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biochemistry, molecular biology, genetics, and the like, which are within the skill of the art. Such techniques are explained fully in the literature.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

All publications and patents cited in this specification are cited to disclose and describe the methods and/or materials in connection with which the publications are cited. Publications and patents that are incorporated by reference, where noted, are incorporated by reference as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. Any terms not specifically defined within the instant application, including terms of art, are interpreted as would be understood by one of ordinary skill in the relevant art; thus, is not intended for any such terms to be defined by a lexicographical definition in any cited art, whether or not incorporated by reference herein, including but not limited to, published patents and patent applications. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

The articles “a,” “an” and “the” are used to refer to one or to more than one (i.e., to at least one) of the grammatical object of the article.

As used herein, “about,” “approximately,” and the like, when used in connection with a numerical variable, generally refers to the value of the variable and to all values of the variable that are within the experimental error (e.g., within the 95% confidence interval for the mean) or within +/−10% of the indicated value, whichever is greater.

The terms “comprise”, “comprising”, “including” “containing”, “characterized by”, and grammatical equivalents thereof are used in the inclusive, open sense, meaning that additional elements may be included. It is not intended to be construed as “consists of only.”

As used herein, “consisting of” and grammatical equivalent thereof exclude any element, step or ingredient not specified in the claim.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “subject” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans).

As used herein “cancer” can refer to one or more types of cancer including, but not limited to, acute lymphoblastic leukemia, acute myeloid leukemia, adrenocortical carcinoma, Kaposi Sarcoma, AIDS-related lymphoma, primary central nervous system (CNS) lymphoma, anal cancer, appendix cancer, astrocytomas, atypical teratoid/Rhabdoid tumors, basa cell carcinoma of the skin, bile duct cancer, bladder cancer, bone cancer (including but not limited to Ewing Sarcoma, osteosarcomas, and malignant fibrous histiocytoma), brain tumors, breast cancer, bronchial tumors, Burkitt lymphoma, carcinoid tumor, cardiac tumors, germ cell tumors, embryonal tumors, cervical cancer, cholangiocarcinoma, chordoma, chronic lymphocytic leukemia, chronic myelogenous leukemia, chronic myeloproliferative neoplasms, colorectal cancer, craniopharyngioma, cutaneous T-Cell lymphoma, ductal carcinoma in situ, endometrial cancer, ependymoma, esophageal cancer, esthesioneuroblastoma, extracranial germ cell tumor, extragonadal germ cell tumor, eye cancer (including, but not limited to, intraocular melanoma and retinoblastoma), fallopian tube cancer, gallbladder cancer, gastric cancer, gastrointestinal carcinoid tumor, gastrointestinal stromal tumors, central nervous system germ cell tumors, extracranial germ cell tumors, extragonadal germ cell tumors, ovarian germ cell tumors, testicular cancer, gestational trophoblastic disease, hary cell leukemia, head and neck cancers, hepatocellular (liver) cancer, Langerhans cell histiocytosis, Hodgkin lymphoma, hypopharyngeal cancer, islet cell tumors, pancreatic neuroendocrine tumors, kidney (renal cell) cancer, laryngeal cancer, leukemia, lip cancer, oral cancer, lung cancer (non-small cell and small cell), lymphoma, melanoma, Merkel cell carcinoma, mesothelioma, metastatic squamous cell neck cancer, midline tract carcinoma with and without NUT gene changes, multiple endocrine neoplasia syndromes, multiple myeloma, plasma cell neoplasms, mycosis fungoides, myelodyspastic syndromes, myelodysplastic/myeloproliferative neoplasms, chronic myelogenous leukemia, nasal cancer, sinus cancer, non-Hodgkin lymphoma, pancreatic cancer, paraganglioma, paranasal sinus cancer, parathyroid cancer, penile cancer, pharyngeal cancer, pheochromocytoma, pituitary cancer, peritoneal cancer, prostate cancer, rectal cancer, Rhabdomyosarcoma, salivary gland cancer, uterine sarcoma, Sézary syndrome, skin cancer, small intestine cancer, large intestine cancer (colon cancer), soft tissue sarcoma, T-cell lymphoma, throat cancer, oropharyngeal cancer, nasopharyngeal cancer, hypoharyngeal cancer, thymoma, thymic carcinoma, thyroid cancer, transitional cell cancer of the renal pelvis and ureter, urethral cancer, uterine cancer, vaginal cancer, cervical cancer, vascular tumors and cancer, vulvar cancer, and Wilms Tumor.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “deoxyribonucleic acid (DNA)” and “ribonucleic acid (RNA)” can generally refer to any polyribonucleotide or polydeoxyribonucleotide, which may be unmodified RNA or DNA or modified RNA or DNA. RNA can be in the form of non-coding RNA such as tRNA (transfer RNA), snRNA (small nuclear RNA), rRNA (ribosomal RNA), anti-sense RNA, RNAi (RNA interference construct), siRNA (short interfering RNA), microRNA (miRNA), or ribozymes, aptamers, guide RNA (gRNA), CRISPR RNA (crRNA), Trans-activating crRNA (tracrRNA), or coding mRNA (messenger RNA).

As used herein, the terms “guide polynucleotide,” “guide sequence,” or “guide RNA” as can refer to any polynucleotide sequence having sufficient complementarity with a target polynucleotide sequence to hybridize with the target sequence and direct sequence-specific binding of a CRISPR complex to the target sequence. The degree of complementarity between a guide polynucleotide and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). A guide polynucleotide (also referred to herein as a guide sequence and includes single guide sequences (sgRNA)) can be about or more than about 5, 10, 11, 12, 13, 14, 15, 16, 17, 18, 19, 20, 21, 22, 23, 24, 25, 26, 27, 28, 29, 30, 35, 40, 45, 50, 75, 90, 100, 110, 112, 115, 120, 130, 140, or more nucleotides in length. The guide polynucleotide can include a nucleotide sequence that is complementary to a target DNA sequence. This portion of the guide sequence can be referred to as the complementary region of the guide RNA. In some contexts, the two are distinguished from one another by calling one the complementary region or target region and the rest of the polynucleotide the guide sequence or tracrRNA. The guide sequence can also include one or more miRNA target sequences coupled to the 3′ end of the guide sequence. The guide sequence can include one or more MS2 RNA aptamers incorporated within the portion of the guide strand that is not the complementary portion. As used herein the term guide sequence can include any specially modified guide sequences, including but not limited to those configured for use in synergistic activation mediator (SAM) implemented CRISPR (Nature 517, 583-588 (29 Jan. 2015) or suppression (Cell Volume 154, Issue 2, 18 Jul. 2013, Pages 442-451). A guide polynucleotide can be less than about 150, 125, 75, 50, 45, 40, 35, 30, 25, 20, 15, 12, or fewer nucleotides in length. The ability of a guide polynucleotide to direct sequence-specific binding of a CRISPR complex to a target sequence may be assessed by any suitable assay. For example, the components of a CRISPR system sufficient to form a CRISPR complex, including the guide polynucleotide to be tested, may be provided to a host cell having the corresponding target sequence, such as by transfection with vectors encoding the components of the CRISPR sequence, followed by an assessment of preferential cleavage within the target sequence. Similarly, cleavage of a target polynucleotide sequence may be evaluated in a test tube by providing the target sequence, components of a CRISPR complex, including the guide polynucleotide to be tested and a control guide polynucleotide different from the test guide polynucleotide, and comparing binding or rate of cleavage at the target sequence between the test and control guide polynucleotide reactions. Other assays are possible, and will occur to those skilled in the art.

As used herein, “nucleic acid,” “nucleotide sequence,” and “polynucleotide” can be used interchangeably herein and can generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphorothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, the terms “optional” or “optionally” means that the subsequently described event or circumstance can or cannot occur, and that the description includes instances where said event or circumstance occurs and instances where it does not.

As used herein, the term “specific binding” or “preferential binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸M or less, 10⁻⁹ M or less, 10⁻¹⁰ M or less, 10⁻¹¹M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

### Overview

In accordance with the purpose(s) of the present disclosure, as embodied and broadly described herein, the present disclosure provides robust CRISPR complex-based detection systems and methods with high sensitivity and specificity. The present disclosure also provides modified crRNAs for use with CRISPR complex-based detection. Embodiments disclosed herein can detect polynucleotides with high sensitivity and specificity. Moreover, the embodiments disclosed herein can be prepared for convenient distribution and point-of-care (POC) applications. Such embodiments are useful in multiple scenarios in human health including, for example, viral detection, bacterial strain typing, sensitive genotyping, and detection of disease-associated cell free DNA.

In one aspect, the present disclosure provides a nuclei acid detection system including a CRISPR-associated (Cas) enzyme with trans cleavage activity; a guide CRISPR RNA (crRNA) including a guide sequence and a polynucleotide extension sequence, wherein the guide sequence is configured to bind to a target polynucleotide, the polynucleotide extension sequence is linked to 3′-end of the guide sequence, and the polynucleotide extension sequence includes a ssDNA or ssRNA with 1-19 nucleotides; and a probe including an oligonucleotide element labeled with a detectable label, wherein a detectable signal or a detectable molecule is generated when the probe is cleaved by the CRISPR-associated (Cas) enzyme.

In one embodiment, the guide sequence is substantially complementary to the target polynucleotide. In another embodiment, the guide sequence is complementary to the target polynucleotide. In one embodiment, the detection system includes two or more crRNA, and two or more guide sequences. In one embodiment, the detection system includes a buffer.

Probe

As used herein, a “probe” refers to a polynucleotide-based molecule that can be cleaved by an activated CRISPR-associated enzyme with a trans-cleavage activity to produce a detectable signal or a detectable molecule. A detectable signal may be any signal that can be detected using optical, fluorescent, chemiluminescent, electrochemical or other detection methods known in the art. The probe comprises an oligonucleotide element. In one embodiment, a first end of the oligonucleotide element in the probe is linked to a fluorophore; and a second end of the oligonucleotide element in the probe is linked to a quencher of the fluorophore. In one embodiment, the probe further comprises biotin.

Quenching of the fluorophore can occur as a result of the formation of a non-fluorescent complex between the fluorophore and another fluorophore or nonfluorescent molecule. This mechanism is known as ground state complex formation, static quenching, or contact quenching. Accordingly, the oligonucleotide element may be designed so that the fluorophore and quencher are in sufficient proximity for contact quenching to occur. Fluorophores and their cognate quenchers are known in the art and can be selected for this purpose by one having ordinary skill in the art. Upon activation of the CRISPR-associated enzyme disclosed herein, the oligonucleotide-based probe is cleaved thereby severing the proximity between the fluorophore and quencher needed to maintain the contact quenching effect. Accordingly, detection of the fluorophore may be used to determine the presence of a target molecule in a sample. In one embodiment, the fluorophore is selected from the group consisting of FITC, HEX and FAM, and the quencher is selected from the group consisting of BHQ1, BHQ2, MGBNFQ, and 3IABkFQ. In one embodiment, a first end of the oligonucleotide element in the probe is linked to a fluorophore; a second end of the oligonucleotide element in the probe is linked to a quencher; and the probe further comprises biotin. In one embodiment, a fluorophore-quencher probe is within the crRNA and the quencher was only cleaved in the presence of a target polynucleotide.

A detectable molecule may be any molecule that can be detected by methods known in the art. In one embodiment, the detectable molecule is one member of a binding pair and can be detected by binding to another member of the binding pair. Examples of binding pairs include, but are not limited to, antibody-antigen pairs, enzyme-substrate pairs, receptor-ligand pairs, and streptavidin-biotin.

In one embodiment, the detectable label is a label selected from the group consisting of FAM-biotin, FITC-biotin, FAM-biotin-quencher, and FITC-biotin-quencher. In one embodiment, a first end of the oligonucleotide element in the probe is linked to FITC, and a second end of the oligonucleotide in the probe is linked to biotin.

In one embodiment, the oligonucleotide element in the probe is ssDNA or RNA. In another embodiment, the oligonucleotide element in the probe is a ssDNA. Since the data in the examples below shows that the CRISPR-Cas system preferentially cleaves A/T rich sequences, in one embodiment, the ssDNA in the probe includes at least 55%, 60%, 65%, 70%, 75%, 80%, 90%, or 95% of A and/or T. In one embodiment, the ssDNA consists of A and/or T. In one embodiment, the oligonucleotide element is TTATT.

In one embodiment, the probe comprises FAM-TTATT-3IABkFQ. In another embodiment, the probe comprises FITC-TTATT-Biotin. In another embodiment, the probe comprises FAM-TTATTA(internal biotin)T-3IABkFQ.

CRISPR-Associated Enzyme

CRISPR-associated enzymes (also known as CRISPR effector protein) are enzymes which can bind to a guide RNA and to a complementary target polynucleotide sequence. Some CRISPR-associated enzymes may possess trans-cleavage activity. In one embodiment, an activated CRISPR-associated enzyme remains active following binding of a target sequence and continues to non-specifically cleave non-target oligonucleotides. This guide molecule-programmed trans-cleavage activity provides an ability to use CRISPR systems to detect the presence of a specific target oligonucleotide to trigger non-specific polynucleotide cleavage that can serve as a readout.

In one embodiment, the CRISPR-associated enzyme is Cas 12, Cas 13, or Cas14. In one embodiment, the CRISPR-associated enzyme is Cas12a, Cas12b, or Cas13a. In another embodiment, the CRISPR-associated enzyme is Cas 12a. In another embodiment, the CRISPR-associated enzyme is one selected from the group consisting of FnCas12a, AsCas12a, LbCas12a, Lb5Cas12a, HkCas12a, OsCas12a, TsCas12a, BbCas12a, BoCas12a, and Lb4Cas12a.

In another embodiment, the CRISPR-associated enzyme is MbCas12a, FnCas12a or LbCas12a. In another embodiment, the CRISPR-associated enzyme is LbCas12a. In one embodiment, the CRISPR-associated enzyme is Cas12 derived from a bacterium of the genus Acidaminococcus or from the genus Lachnospiraceae.

crRNA

The present disclosure provides a modified guide CRISPR RNA (crRNA) including a guide sequence and a polynucleotide extension sequence, wherein the guide sequence is configured to bind to a target polynucleotide, the polynucleotide extension sequence is linked to 3′ end of the guide sequence, and the polynucleotide extension sequence includes a ssDNA or ssRNA with 1-19 nucleotides. In one embodiment, the detection system disclosed herein includes at least two crRNAs. Each of the crRNAs includes a guide sequence and a polynucleotide extension sequence. In one embodiment, the guide sequences in the at least two crRNA are different, and each guide sequence might bind to a different target polynucleotide.

In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In one embodiment, the degree of complementarity between a guide sequence and its corresponding target sequence is about or more than about 50%, 60%, 75%, 80%, 85%, 90%, 95%, 97.5%, 99%, or more.

In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In one embodiment, the guide sequence is 10-30 nucleotides long.

In one embodiment, the polynucleotide extension sequence includes a ssDNA or ssRNA with 1-19 nucleotides. In one embodiment, the polynucleotide extension sequence includes an RNA. In one embodiment the polynucleotide extension sequence includes a ssDNA. In an embodiment, the polynucleotide extension sequence comprises 3-15 nucleotides.

In one embodiment, the polynucleotide extension sequence includes 4-13 nucleotides. In another embodiment, the polynucleotide extension sequence includes 5-9 nucleotides. In another embodiment, the polynucleotide extension sequence includes 7 nucleotides. In one embodiment, the polynucleotide includes 1, 2, 3, 4, 5, 6, 7, 8, 9, 10, 11, 12, 13, 14, 15, 16, 17, 18, or 19 nucleotides. In one embodiment, the polynucleotide extension sequence has a sequence of T ATTATT (residues 42-48 of SEQ ID NO:22). In another embodiment, the polynucleotide extension sequence has a sequence of TATTATTATTATT (residues 42-54 of SEQ ID NO:23). In another embodiment, the polynucleotide extension sequence has a sequence of (residues 42-60 of SEQ ID NO:24). In another embodiment, the polynucleotide extension sequence has a sequence of CGCCGCC (residues 42-48 of SEQ ID NO:26) In one embodiment, the system includes two guide CRISPR RNAs (crRNA) and two polynucleotide targets, wherein a first crRNA including a first guide sequence configured to bind to a first target polynucleotide; and a second crRNA including a second guide sequence configured to bind to a second target polynucleotide. In embodiments, the first and second target polynucleotides can be different sequences associated with the same condition, such as cancer, an infectious disease, and the like. For instance, the first target polynucleotide and second target polynucleotide can be different sequences from the same virus to improve accurate detection of the virus. In embodiments, the first target polynucleotide is a DNA fragment synthesized from a RNA fragment in N1 region of SARS-CoV-2, and the second target polynucleotide is a DNA fragment synthesized from a RNA fragment in N2 region of SARS-CoV-2.

In one embodiment, the crRNA further includes a linker having two ends, one end of the linker linked to 3′-end of the polynucleotide extension sequence, and the other end of the linker linked to a complementary sequence, wherein the complementary sequence is complementary to the extension sequence (the complementary sequence may optionally be complementary to the extension sequence and a portion of the guide sequence), wherein the extension sequence, the linker, and the complementary sequence form a toehold conformation, and the toehold conformation unfolds when the guide sequence binds to the target polynucleotide.

In another embodiment, the polynucleotide extension sequence includes a linker having two ends, one end of the linker linked to 3′-end of the guide sequence, and the other end of the linker linked to a complementary sequence, wherein the complementary sequence is complementary to at least a 3′ portion of the guide sequence, wherein the linker, the complementary sequence, and the 3′ portion of the guide sequence form a toehold conformation, and the toehold conformation unfolds when the guide sequence binds to the target polynucleotide.

Target Polynucleotide

In the context of formation of a CRISPR complex, “target polynucleotide” refers to a polynucleotide to which a guide sequence is designed to have complementarity, where hybridization between a target sequence and a guide sequence promotes the formation of a CRISPR complex.

A target sequence may include ssDNA, RNA, and/or heteroduplex RNA/DNA. The term “target DNA” refers to a DNA polynucleotide being or comprising the target polynucleotide. In other words, the target DNA may be a DNA polynucleotide or a part of a DNA polynucleotide to which a part of the guide RNA, i.e. the guide sequence, is designed to have complementarity and to which the function mediated by the complex comprising CRISPR-associated enzyme and a gRNA is to be directed.

In some embodiments, a target polynucleotide is located in the nucleus or cytoplasm of a cell.

In one embodiment, a target polynucleotide is diagnostic for a disease state. In one embodiment, the disease state is cancer. In another embodiment, the disease state is a genetic disease or disorder. In another embodiment, the disease state is an infection. In one embodiment, the infection is caused by a virus, a bacterium, a fungus, protozoan, or a parasite. In one embodiment, the infection is a viral infection. In one embodiment, the infection is caused by SARS-CoV-2.

In one embodiment, the target polynucleotide includes a modified nucleotide. In another embodiment, the modified nucleotide is a methylated nucleotide.

In one embodiment, the target polynucleotide includes a DNA fragment synthesized from an RNA fragment in N1 region of SARS-CoV-2. In another embodiment, the target polynucleotide includes a DNA fragment synthesized from a RNA fragment in N2 region of SARS-CoV-2. In another embodiment, the target polynucleotide is at least one selected from the group consisting of Control genes: RNase P (P1 & P2), GFP, HIV Tat gene, HCV gene encoding a polyprotein precursor PCA3 (Prostate cancer Antigen 3), and SARS-CoV-2 genes: N (N1 & N2), E gene (E1 & E2), and R (R1 & R2).

In one embodiment, the detection system disclosed here may bind to at least two target polynucleotides. In embodiments, as mentioned above, the detection system disclosed here may include one or more different modified crRNAs, each configured to bind to a different target polynucleotide.

Amplification of a Target Polynucleotide

In one embodiment, target RNAs and/or DNAs may be amplified prior to activating the CRISPR-associated enzyme. Any suitable RNA or DNA amplification technique may be used. In another embodiment, the RNA or DNA amplification is an isothermal amplification. In another embodiment, the isothermal amplification may be nucleic acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR).

In another embodiment, non-isothermal amplification methods may be used which include, but are not limited to, PCR, multiple displacement amplification (MDA), rolling circle amplification (RCA), ligase chain reaction (LCR), or ramification amplification method (RAM).

In another embodiment, the RNA or DNA amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. The NASBA reaction has the additional advantage of being able to proceed under moderate isothermal conditions, for example at approximately 40° C., making it suitable for systems and devices deployed for early and direct detection in the field and far from clinical laboratories.

In another embodiment, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In another embodiment, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the CRISPR system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds.

Accordingly, in one embodiment, the systems disclosed herein may include amplification reagents. Different components or reagents useful for amplification of nucleic acids may be included. For example, an amplification reagent may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1M, or the like. One of skill in the art will be able to determine an appropriate concentration of a buffer such as Tris for use with the present invention.

In one embodiment, the amplification reagent is for an amplification selected from the group consisting of nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), and ramification amplification method (RAM).

In another embodiment, the amplification reagent is for an amplification selected from the group consisting of nucleic acid sequence-based amplification (NASBA), recombinase polymerase amplification (RPA), and loop-mediated isothermal amplification (LAMP).

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH4)2SO4], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application, and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

It will be evident that detection methods of the invention can involve nucleic acid amplification and detection procedures in various combinations. The nucleic acid to be detected can be any naturally occurring or synthetic nucleic acid, including but not limited to DNA and RNA, which may be amplified by any suitable method to provide an intermediate product that can be detected. Detection of the intermediate product can be by any suitable method including but not limited to binding and activation of a CRISPR-associated enzyme which produces a detectable signal moiety by trans-cleavage activity.

Divalent Ion

In one embodiment, the nucleic acid detection system includes a divalent metal cation. A divalent metal cation may improve sensitivity of the CRISPR-associated system. In one embodiment, the divalent metal cation is magnesium. In one embodiment, the detection solution has Mg²⁺ at the concentration of 1-50 mM, 3-40 mM, 5-35 mM, 7-25 mM, 9-20, or 10-15 mM. In another embodiment, the detection solution has Mg²⁺ at the concentration of about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM. In another embodiment, the detection solution has Mg²⁺ at the concentration of about 13 mM.

In another embodiment, the divalent cation is calcium. In one embodiment, the detection solution has Ca²⁺ at the concentration of 1-50 mM, 3-40 mM, 5-35 mM, 7-25 mM, 9-20, or 10-15 mM. In another embodiment, the detection solution has Ca²⁺ at the concentration of about 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, or 20 mM.

Kits

In one aspect, the present disclosure provides a kit for detecting a virus. The kit includes the detection system disclosed herein, and instructions for use of the detection system for detecting the virus. In one embodiment, the kit includes a test strip for detecting a detectable molecule. In one embodiment, the test strip comprises a colorimetric paper-based assay or an electrochemical biosensor.

In another aspect, the present disclosure provides a kit for detecting a target polynucleotide. The kit includes the detection system disclosed herein and instructions for use of the detection system for detecting the target polynucleotide.

Lateral Flow Device/Strip

In one aspect, the present disclosure provides a lateral flow device. The lateral flow device includes a sample loading portion; and the detection system disclosed herein. In one embodiment, the sample loading portion includes an amplification reagent. The amplification reagent may amplify the target polynucleotide.

Substrates suitable for use in lateral flow assays or devices are known in the art. These may include, but are not limited to, membranes or pads made of cellulose and/or glass fiber, polyesters, nitrocellulose, or absorbent pads.

In one embodiment, the lateral flow strip further includes a first capture line, optionally a horizontal line running across the device. The first capture region is proximate to and on the same end of the lateral flow substrate as the sample loading portion. A first binding agent that specifically binds the first molecule of the probe is fixed or otherwise immobilized to the first capture region. The second capture region is located towards the opposite end of the lateral flow substrate from the first binding region. A second binding agent is fixed or otherwise immobilized at the second capture region. The second binding agent specifically binds the second molecule of the probe, or the second binding agent may bind a detectable ligand. For example, the detectable ligand may be a particle, such as a colloidal particle, that when it aggregates can be detected visually. The particle may be modified with an antibody that specifically binds the second molecule on the probe. If the probe is not cleaved it will facilitate accumulation of the detectable ligand at the first binding region. If the probe is cleaved the detectable ligand is released to flow to the second binding region. In such an embodiment, the second binding agent is an agent capable of specifically or non-specifically binding the detectable ligand on the antibody on the detectable ligand. Examples of suitable binding agents for such an embodiment include, but are not limited to, protein A and protein G.

Lateral support substrates may be located within a housing. The housing may include at least one opening for loading samples and a second single opening or separate openings that allow for reading of detectable signal generated at the first and second capture regions.

The CRISPR detection system disclosed herein may be freeze-dried to the lateral flow substrate and packaged as a ready to use device, or the detection system may be added to the reagent portion of the lateral flow substrate at the time of using the device. Samples to be screened are loaded at the sample loading portion of the lateral flow substrate. The samples must be liquid samples or samples dissolved in an appropriate solvent, usually aqueous. The liquid sample begins to flow from the sample portion of the substrate towards the first and second capture regions.

In one embodiment, the lateral flow strip includes two capture regions. The intact probe is bound at the first capture region by binding between the first binding agent and a first molecule in the probe. When target molecule(s) are not present in the sample, the CRISPR-associated enzyme trans-cleavage activity is not activated. Then the probe is not cleaved. Because the probe is not cleaved, the second molecule of the probe does not flow to the second capture region. If target molecule(s) are present in the sample, the CRISPR-associated enzyme trans-cleavage activity is activated. As activated CRISPR-associated enzyme comes into contact with the bound probe, the probe is cleaved, releasing the second molecule to flow further down the lateral flow substrate towards the second binding region. The released second molecule is then captured at the second capture region by binding to the second binding agent, where additional detection agent may also accumulate by binding to the second molecule. Accordingly, if the target molecule(s) is not present in the sample, a detectable signal will appear at the first capture region, and if the target molecule(s) is present in the sample, a detectable signal will appear at the location of the second capture region.

In embodiments the system can also utilize commercially available test strips, such as pregnancy and ovulation test strips. In such an embodiment, the system can include probes detectable by a commercial test strip. In an embodiment, the probe includes a detectable molecule, such as human chorionic gonadotropin (hCG), such that upon cleavage of the probe, the hCG is detectable in a commercially available pregnancy test. In an embodiment the probe includes human luteinizing hormone (LH), such that upon cleavage of the probe, the LH is detectable in a commercially available ovulation test strip. In another embodiment, the probe can include glucose, such that upon cleavage of the probe, the glucose can be detected by commercially available glucose meters.

Ribonucleoprotein Complex

In one aspect, the present disclosure provides a ribonucleoprotein complex. The ribonucleoprotein complex includes a CRISPR-associated enzyme with trans-cleavage activity; and a guide CRISPR RNA (crRNA) including a guide sequence and a polynucleotide extension sequence, wherein the guide sequence is configured to bind to a target polynucleotide, the polynucleotide extension sequence is linked to 3′-end of the guide sequence, and the polynucleotide extension sequence includes a ssDNA or ssRNA with 1-19 nucleotides. In one embodiment, the CRISPR-associated enzyme is Cas12a. In one embodiment, the CRISPR-associated enzyme is MbCas12a, FnCas12a, or LbCas12a.

In one embodiment, the crRNA further includes a linker having two ends, one end of the linker linked to 3′-end of the polynucleotide extension sequence, and the other end of the linker linked to a complementary sequence, wherein the complementary sequence is complementary to the extension sequence, wherein the extension sequence, the linker, and the complementary sequence form a toehold conformation, and the toehold conformation unfolds when the guide sequence binds to the target polynucleotide.

In another embodiment, the polynucleotide extension sequence includes a linker having two ends, one end of the linker linked to 3′-end of the guide sequence, and the other end of the linker linked to a complementary sequence, wherein the complementary sequence is complementary to at least a 3′ portion of the guide sequence, wherein the linker, the complementary sequence, and the 3′ portion of the guide sequence form a toehold conformation, and the toehold conformation unfolds when the guide sequence binds to the target polynucleotide.

Methods of Use of the CRISPR Complex-Based Detection System

In one embodiment, the methods described herein may involve targeting one or more polynucleotide targets of interest. The polynucleotide targets of interest may be targets which are relevant to a specific disease or the treatment thereof, relevant for the generation of a given trait of interest or relevant for the production of a molecule of interest. When referring to the targeting of a “polynucleotide target” this may include targeting one or more of a coding region, an intron, a promoter and any other 5′ or 3′ regulatory regions such as termination regions, ribosome binding sites, enhancers, silencers etc. The gene may encode any protein or RNA of interest. Accordingly, the target may be a coding region which can be transcribed into mRNA, tRNA or rRNA, but also recognition sites for proteins involved in replication, transcription and regulation thereof.

In one aspect, the present disclosure provides a method of detecting a target polynucleotide in a sample obtained from a subject. The method includes the steps of: contacting the sample with the detection system disclosed herein, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and detecting the signal or the molecule, wherein detection of the signal or the molecule indicates presence of the target polynucleotide in the sample.

In one embodiment, the method further includes amplification of the target polynucleotide or a polynucleotide that the target polynucleotide is derived from. In another embodiment, the method does not include a step of amplification. In one embodiment, the guide sequence is complementary to the to the target polynucleotide.

In one embodiment, the detection system is on a substrate, and wherein the substrate is exposed to the sample. In another embodiment, the substrate includes a flexible material. In one embodiment, the substrate is selected from the group consisting of: a paper substrate, a fabric substrate, and a polymer-based substrate.

In one embodiment, the sample includes saliva, respiratory secretions, exudate, blood, plasma, urine, stool, and/or sera. In one embodiment, the method includes amplifying the target polynucleotide in the sample prior to contacting the sample with the detection system. In one embodiment, amplifying the target polynucleotide includes isothermal amplification. In one embodiment, the detectable molecule is detected by a test strip.

In another aspect, the present disclosure provides a method of detecting a condition in a subject. The method includes the steps of: contacting a sample from the subject with the detection system disclosed herein, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and detecting the signal or the molecule, wherein detection of the signal or the molecule indicates presence of the condition.

In one embodiment, the sample is urine, stool, sera, blood, respiratory secretions, exudate, and/or saliva. In another embodiment, the sample and the detection system are incubated at a temperature of 20-60° C., 30-60° C., 40-60° C., 40-55° C. for a period of time. The detection may be performed in one-pot or multiple-pot forms.

In another embodiment, the sample and the detection system are incubated at a temperature of about 40, 41, 42, 43, 44, 45, 46, 47, 48, 49, 50, 51, 52, 53, 54, 55° C. for a period of time. In another embodiment, detecting the signal or the molecule occurs within 5, 10, 15, 20, 25, 30, 35, 40, 45, 50, or 60 minutes of contacting the sample with the detection system.

In one aspect, the present disclosure provides methods of detecting a target polynucleotide in a sample obtained from a subject. The method includes the steps of contacting a sample from the subject with a detection system, wherein the detection system comprises a CRISPR-associated (Cas) enzyme with trans cleavage activity, a guide CRISPR RNA (crRNA) comprising a guide sequence (with or without an extension sequence), and a probe comprising an oligonucleotide element labeled with a detectable label, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and detecting the signal or the molecule, wherein detection of the signal or the molecule indicates presence of the target polynucleotide in the sample; wherein the sample and the detection system is incubated at a temperature of 30-70° C. for a period of time.

In one embodiment, the combination of the sample and the detection system is incubated at a temperature of 30-60° C. for a period of time. In another embodiment, the combination of the sample and the detection system is incubated at a temperature of 35-60° C. for a period of time. In another embodiment, the combination of the sample and the detection system is incubated at a temperature of 40-55° C. for a period of time. In another embodiment, the combination of the sample and the detection system is incubated at a temperature of 40-45° C. for a period of time. In another embodiment, the combination of the sample and the detection system is incubated at a temperature of 45-50° C. for a period of time. In another embodiment, the combination of the sample and the detection system is incubated at a temperature of 50-55° C. for a period of time. In another embodiment, the combination of the sample and the detection system is incubated at a temperature of about 40, 41, 42, 43, 44, 45, 46, 47, 48, 49, 50, 51, 52, 52, 54, 55, 56, 57, 58, 59, 60° C.

Additional details regarding the methods and compositions, of the present disclosure are provided in the Examples below. The specific examples below are to be construed as merely illustrative, and not limitative of the remainder of the disclosure in any way whatsoever. Without further elaboration, it is believed that one skilled in the art can, based on the description herein, utilize the present disclosure to its fullest extent.

It should be emphasized that the embodiments of the present disclosure, particularly, any “preferred” embodiments, are merely possible examples of the implementations, merely set forth for a clear understanding of the principles of the disclosure. Many variations and modifications may be made to the above-described embodiment(s) of the disclosure without departing substantially from the spirit and principles of the disclosure. All such modifications and variations are intended to be included herein within the scope of this disclosure and protected by the following claims.

The following examples are put forth so as to provide those of ordinary skill in the art with a complete disclosure and description of how to perform the methods and use the compositions and compounds disclosed herein. Efforts have been made to ensure accuracy with respect to numbers (e.g., amounts, temperature, etc.), but some errors and deviations should be accounted for.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The present examples demonstrate that engineered crRNAs and optimized conditions could be used to detect various clinically-relevant nucleic acid targets with higher sensitivity, achieving a limit of detection of in femtomolar range with or without any target pre-amplification step. By extending the 3′-end of the crRNA with different lengths of ssDNA, ssRNA, and phosphorothioate ssDNA, a new self-catalytic behavior and an augmented rate of LbCas12a-mediated collateral cleavage activity as high as 3.5-fold compared to the wild-type crRNA and with up to 8.8-fold improvement in specificity for target recognition were discovered. Particularly, the 7-mer DNA extension to crRNA was determined to be universal and spacer-independent for enhancing the sensitivity and specificity of LbCas12a-mediated nucleic acid detection. This system with engineered crRNAs, reporters, and other conditions is referred to herein as “CRISPR ENHANCE” or just “ENHANCE.” Detailed characterization of the engineered ENHANCE system with various crRNA modifications, target types, reporters, and divalent cations was performed. With isothermal amplification of SARS-CoV-2 RNA using RT-LAMP, the modified crRNAs were incorporated in a paper-based lateral flow assay that could detect the target with up to 23-fold higher sensitivity within 40-60 minutes.

The 3′- and 5′-end of the crRNA were extended, and an amplified trans-cleavage activity and improved specificity of LbCas12a was discovered when the 3′-end is extended with DNA or RNA. This modified crRNA/LbCas12a system was used with the optimal conditions to detect PCA3 in simulated urine with high sensitivity. This ENHANCE technology was used to detect the DNA/RNA heteroduplex and methylated DNA with unprecedented sensitivity. This system was employed to test a range of target nucleic acids, including ssDNA, dsDNA, and RNA from HIV, HCV, and SARS-CoV-2 without the need for further optimization. Compared to wild type CRISPR, the ENHANCE demonstrated enhanced detection of SARS-CoV-2 genomic RNA with improved sensitivity and specificity in a fluorescence-based assay and a paper-based lateral flow assay. These findings are a crucial step towards enhancing detection of nucleic acids and assisting in the diagnosis of various diseases including the COVID-19.

The system allows for additional isothermal amplification methods to be utilized which require temperatures above 37° C. The system will be highly sensitive as well as specific. Everything can be mixed in a single pot a single step test. The detection can be done using fluorescence measurement, fluorescence visualization, paper-based test, electrochemical test, or other platforms. The system can be tailored to be activated in the presence of a DNA or RNA and can be extended to a small molecule or protein target. Various CRISPR/Cas systems can be combined and multiplexed. All these systems can be incorporated with a variety of reporter systems based on either or combination of fluorescence, luminescence, color change, product formation, redox reaction, pH change, surface reaction or cleavage, change in electrical conductivity, resistance, or impedance.

### Example 1. Methods and Materials

### Example 1.1. DNA Activator Preparation

Multiple DNA activators were used in this study. The GFP fragment (942 bp) was produced by amplifying the pEGFP-C1 plasmid using polymerase chain reaction in the Proflex PCR system (ThermoFisher Scientific). The PCR product was purified using Monarch® Nucleic Acid Purification Kit (New England Biolabs Inc.).

Additionally, the 40-nt ds-GFP, ds-PCA3, ds-HCV, ds-HIV, ds-CoV2, and their respective mutants activators were generated by annealing two single-stranded TS and NTS fragments at a 1:1 ratio (Integrated DNA Technologies Inc.) in 1× hybridization buffer (20 nM Tris-Cl, pH 7.8, 100 mM KCl, 5 mM MgCl₂). The annealing process was executed in the Proflex PCR system at 90° C. for 2 minutes followed by gradual cooling to 25° C. at a rate of 0.1° C./s.

### Example 1.2. LbCas12a Expression and Purification

The plasmid LbCpf1-2NLS (Addgene #102566, a gift from Jennifer Doudna Lab)³² was transformed into Nico21(DE3) competent cells (New England Biolabs). Colonies were picked and inoculated in Terrific Broth at 37° C. until OD600=0.6. IPTG was then added to the cultures, and they were grown at 18° C. overnight.

Cell pellets were collected from the overnight cultures by centrifugation, resuspended in lysis buffer (2M NaCl, 20 mM Tris-HCl, 20 mM imidazole, 0.5 mM TCEP, 0.25 mg/ml lysozyme, and 1 mM PMSF, PH=8), and broken by sonication. The sonicated solution then underwent high speed centrifugation at 40,000 RCF for 45 minutes. The collected supernatant was then run through a Ni-NTA Hispur column (Thermofisher) pre-equilibrated with wash buffer A (2M NaCl, 20 mM Tris-HCl, 20 mM imidazole, 0.5 mM TCEP, PH=8). The column was then eluted with buffer B (2M NaCl, 20 mM Tris-HCl, 300 mM imidazole, 0.5 mM TCEP, PH=8). The eluted fractions were then pooled together and underwent TEV cleavage overnight at 4° C. (TEV protease was purified using the plasmid pRK793, #8827 from Addgene, a gift from David Waugh Lab).

The resulting fraction was equilibrated with buffer C (100 mM NaCl, 20 mM HEPES, 0.5 mM TCEP, PH=8) at a 1:1 ratio and run through Hitrap Heparin HP 1 ml column (GE Biosciences). The column was washed with buffer C and gradually eluted at a gradient rate with buffer D (100 mM NaCl, 20 mM HEPES, 0.5 mM TCEP, PH=8). The eluted fraction was concentrated down to 500 μL and passed through the Hiload Superdex 200 pg column (GE Biosciences). The purified LbCas12a was then buffer exchanged with storage buffer (500 mM NaCl, 20 mM Na₂CO₃, 0.1 mM TCEP, 50% glycerol, PH=6) and flash frozen at −80° C. until use.

### Example 1.3. Biolayer Interferometry (BLI) Binding Kinetic Assay

The BLI Ni-NTA biosensors were purchased from Fortebio to perform the binding kinetic study with polyhistidine-tagged LbCas12a. In detail, the experiment was carried out in a 96-well plate and included five steps: baseline, loading, baseline2, association, and dissociation. The biosensors were dipped into the baseline containing 1× kinetic buffer (1×PBS, 0.1% BSA, and 0.01% Tween 20). They were then transferred into each loading well containing 10 g/ml of LbCas12a. After processing through loading and baseline2, the protein-tagged biosensor was next allowed to dip into the crRNA sample wells at different dilution (10, 5, 2.5, 1.25, 0.625, 0.3125, 0.15625, and 0 μg/ml) in the association step. The dissociation step occurred when the biosensors were transferred back to baseline2 at a shake speed of 1000 rpm. All the samples were read by the Octet QKe system (Fortebio). K_{d} was determined by software Data Analysis 10.0 (Fortebio), and only K_{d} with R²>0.9 were extracted for comparison between crRNA wild type and modified crRNAs.

### Example 1.4. Cis-Cleavage Assay

In-vitro digestion reactions were carried out with three different types of the Cas12a family (LbCas12a, AsCas12a, and FnCas12a were purchased from New England Biolabs Inc. or purified in lab, Integrated DNA Technologies Inc., and Abm®, respectively) and a wide array of modified crRNA's (purchased from DNA Technologies Inc.). Cas12a and crRNA were mixed with 1:1 ratio (100 nM:100 nM) in 1× NEBuffer 2.1 and pre-incubated at 25° C. for 15 minutes to promote the ribonucleoprotein complex formation. DNA activator (final concentration of 7 nM) was then added to the mixture to produce a total volume of 30 μL and incubated at 37° C. for 30 minutes.¹⁹ The sample was then analyzed in either 1% agarose gel (for GFP fragments amplified from the pEGFP-C1 plasmid) pre-stained with either SYBER Gold (Invitrogen), GelRed (Biotium Inc.), or premade 15% Novex™ TBE-Urea Gel (Invitrogen).

### Example 1.5. M13mp18 Nonspecific Cleavage Assay

Nonspecific cleavage activity of Cpf1 was activated by incubating Cpf1, crRNA, and DNA activator with a concentration of 100 nM:100 nM: 2 nM in 1× NEBuffer 2.1 buffer at 37° C. for 30 minutes. M13mp18 was then added to the 30 μL reaction mixture and incubated for an additional 45 minutes. A fraction of the reaction was taken out every 5 minutes, quenched in 6× purple gel loading dye (New England Biolabs Inc.), and subsequently analyzed in 1% agarose gel (Fisher Scientific)′.

### Example 1.6. Trans-Cleavage Reporter Assay

The fluorophore-quencher reporter assay was carried out following a standard clinical detection protocol. The Cas12a-crRNA ribonucleoprotein complex was assembled by mixing 100 nM Cas12a and 100 nM crRNA in 1× NEBuffer 2.1 in the Proflex PCR system (ThermoFisher Scientific) at 25° C. for 15 minutes (volume 28.5 μL). The activator (1 nM final concentration), FQ reporter (500 nM final concentration), and UltraPure™ DNase/RNase-Free distilled water (Invitrogen) were pre-added to a 96-well plate (Greiner Bio-One) to a volume of 71.5 μL. The reaction was initiated by adding the Cas12a-crRNA mixture to the 96-well plate preloaded with activator and FQ reporter (Integrated DNA Technologies Inc). The plate was quickly transferred to a plate reader (ClarioStar or BioTek), and fluorescence intensity was measured every 3 minutes for 3 or 12 hours (detection limit assay) (FAM FQ: λₑₓ: 483/30 nm, λₑₘ: 530/30 nm; HEX: λₑₓ: 430/20 nm, λₑₘ: 555/30 nm). After 3 or 12 hours (detection limit assay), the sample was scanned for images using the Amersham Typhoon (GE Healthcare).

For Michaelis-Menten kinetic study, 30 nm LbCas12a: 30 nM crRNA: 1 nM activator were mixed in NEBuffer 2.1 and incubated at 37° C. for 30 minutes. The reaction mixture was then transferred to a 96-well plate (Greiner Bio-One) preloaded with different concentrations of FQ reporter (HEXor FAM FQ reporter: 0 M, 0.05 μM, 0.1 μM, 0.25 μM, 0.5 μM, and 1 μM) and UltraPure™ DNase/RNase-Free distilled water (Invitrogen)′.

To find limit of detection (LoD), the fluorophore-quencher reporter assay was carried out with various concentrations of activator. The LoD calculations were based on the following formula:

$$LoD = \frac{3.3 * std of RFU in the absence of activator}{slope of RFU vs . Activator concentration}$$

### Example 1.7. Effects of Metal Ions on Cas12a Cleavage Study

The metal ions (Mg²⁺, Zn²⁺, Mn²⁺, Cu²⁺, Co²⁺, Ca²⁺) were prepared by diluting chloride salt in different concentrations. For cis-cleavage, the Cas12a-crRNA-metal iron duplex was mixed with 100 nM: 100 nM: varying nM ratio in 1× annealing buffer (100 mM Tris-HCl, pH 7.9 @ 25° C., 500 mM NaCl, 1 mg/ml BSA) and pre-incubated at 25° C. for 15 minutes. DNA activator (GFP or PCA3 fragments) was then added to the mixture to a total volume of 30 μL and incubated at 37° C. for 30 minutes.

### Example 1.8. Paper Strip Test

To minimize the testing time, the following reagent was assembled in a one-pot reaction:
a. 10× NEBuffer: 5 (1× final concentration)
b. 3 μM LbCas12a: 1 (60 nM final concentration)
c. 3 μM crRNA: 2 (120 nM final concentration)
d. 5 μM FAM-biotin reporter: 1.5 μL (150 nM final concentration)
e. Various concentration of Activator: 2
f Nuclease-free water: 38.5 μL (total reaction volume=50 μL)

The reaction mixture was incubated at either 37° C. or 25° C. for different time periods (5, 10, and 20 minutes). A Milenia HybriDetect (TwistDx) strip was dipped in each reaction and allowed for rapid visualization.

For experiment involving a recombinase polymerase amplification (RPA) step, the reaction mix was prepared in the following order:
a. Forward primer (10 μM): 2.4 μL
b. Reverse primer (10 μM): 2.4 μL
c. Primer free rehydration buffer: 29.5 μL
d. Template and nuclease-free water: 13.2 μL
e. (CH₃COO)₂Mg (280 mM): 2.5 μL (total volume=50 μL)

The RPA reaction was incubated at 39° C. for 20-30 minutes prior to LbCas12a reaction.

For experiments involving a RT-LAMP preamplification step of target RNA, the mixture was prepared in the following order (except for the RNA and primer mix samples (IDT Technologies), everything was purchased from New England Biolabs):
a. 10× isothermal amplification buffer: 2.5 μL
b. 100 mM MgSO₄: 1 μL
c. 10 mM dNTP: 3.5 μL
d. 20× primer mix (4 μM F3, 4 μM B3, 32 μM FIP, 32 μM BIP, 8 μM LF and 8 μM BF):
1.25 μL
e. Bst 2.0 polymerase: 1 μL
f Warmstart RTx: 0.5 μL
g. RNase inhibitor, murine (40,000 U/ml): 0.625 μL
h. Nuclease-free water: 9.625 μL
i. RNA sample: 5 (total volume=25 μL)
   The RT-LAMP reaction was incubated at 63° C. for 20-30 minutes prior to LbCas12a reaction.

### Example 2. The Trans-Cleavage Activity is Drastically Improved by 7-Mer ssDNA Extensions to the 3′-End of crRNA

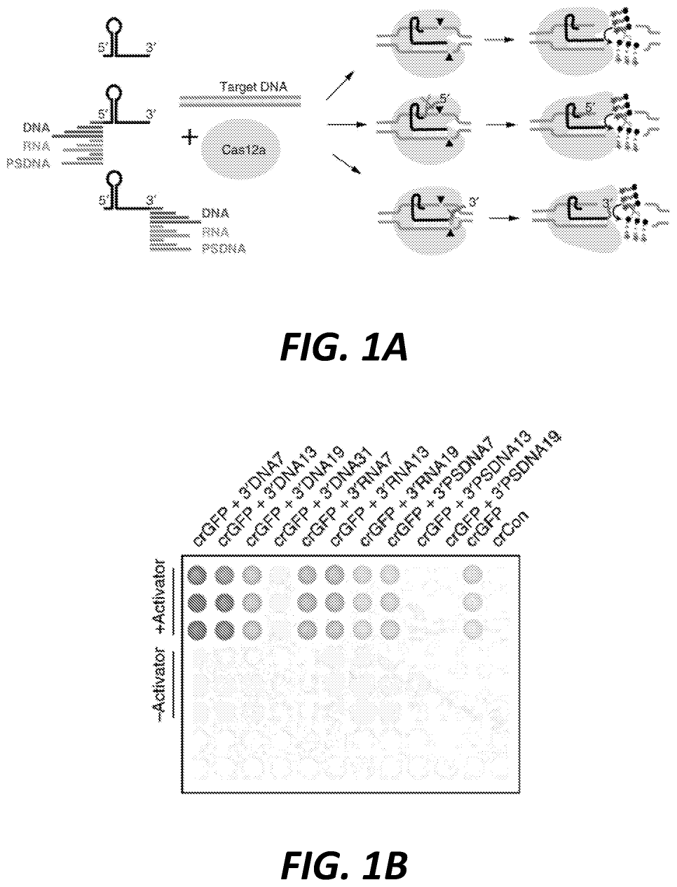

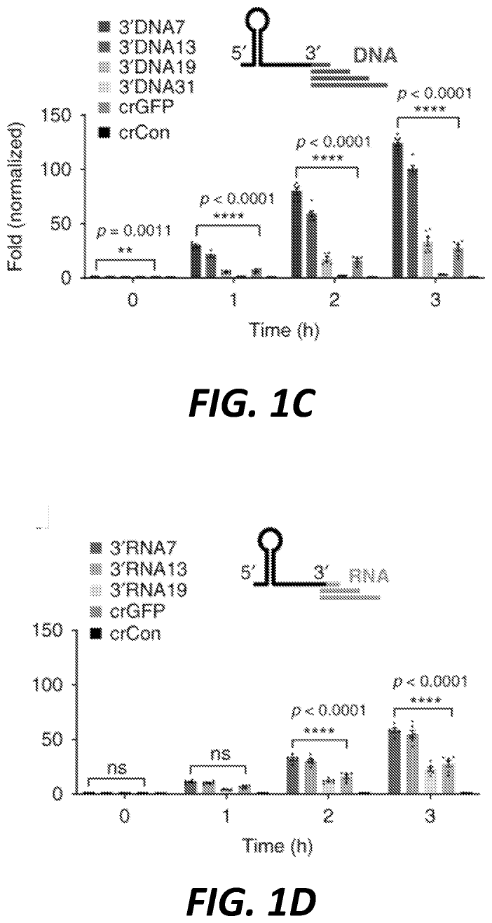

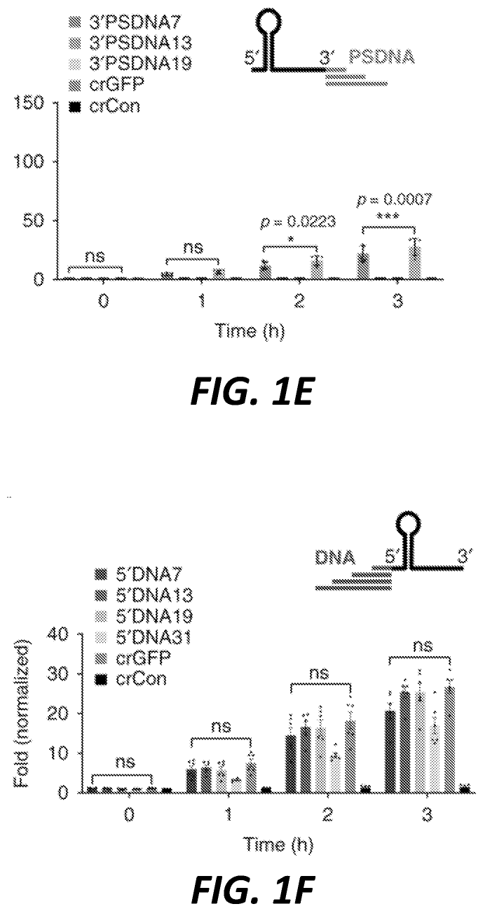

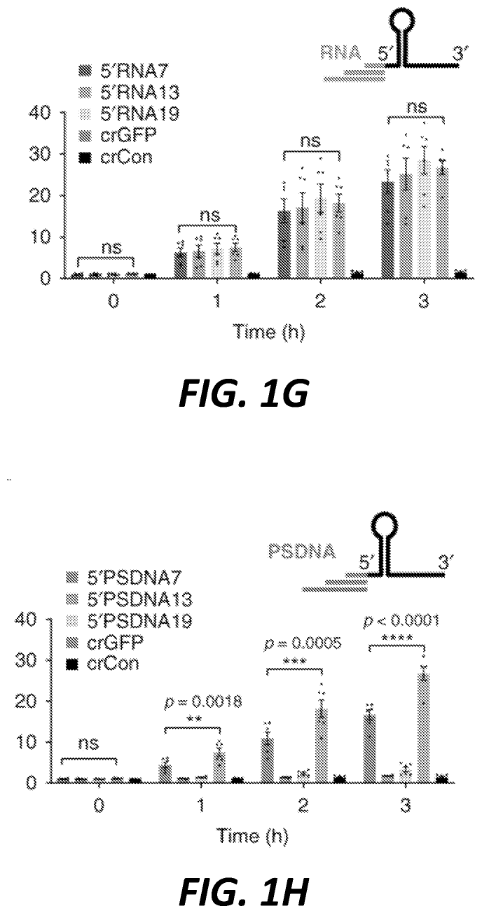

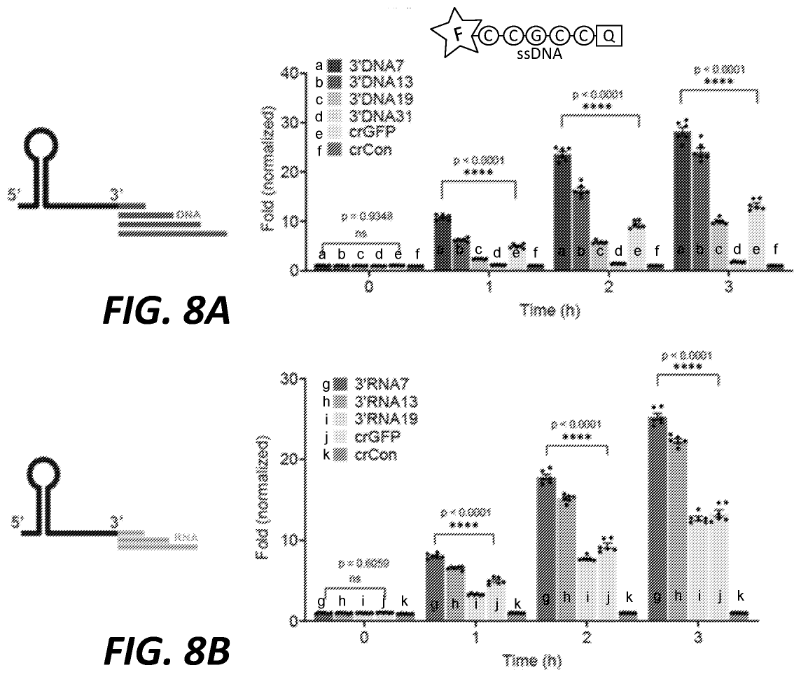

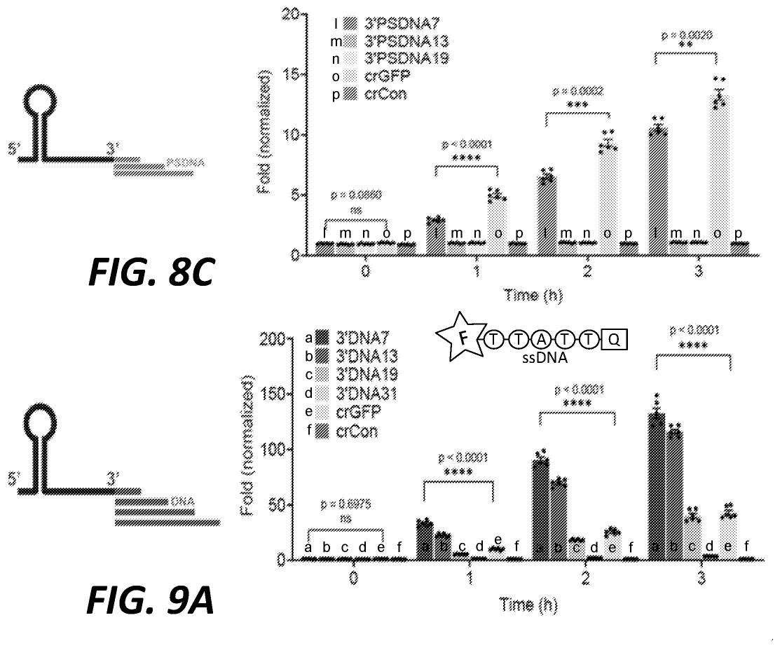

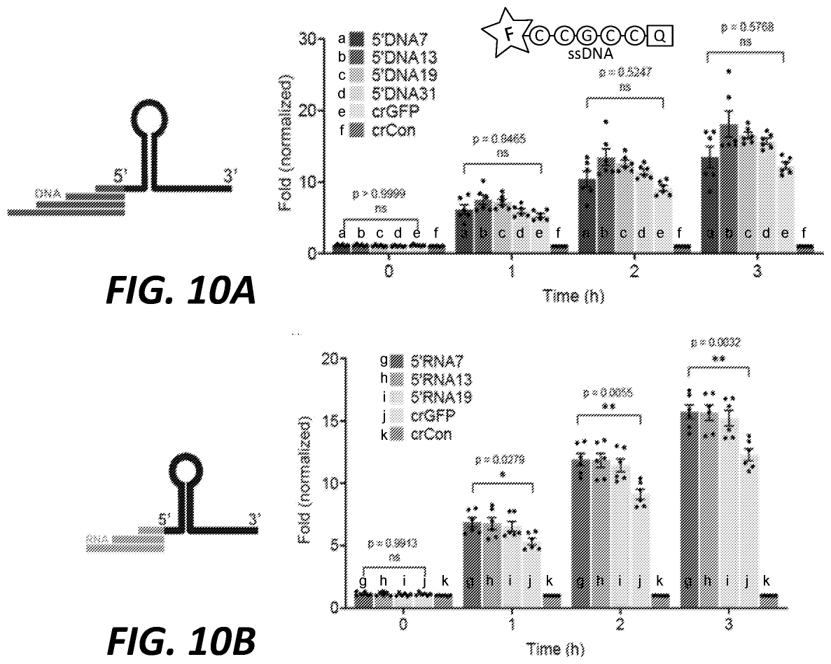

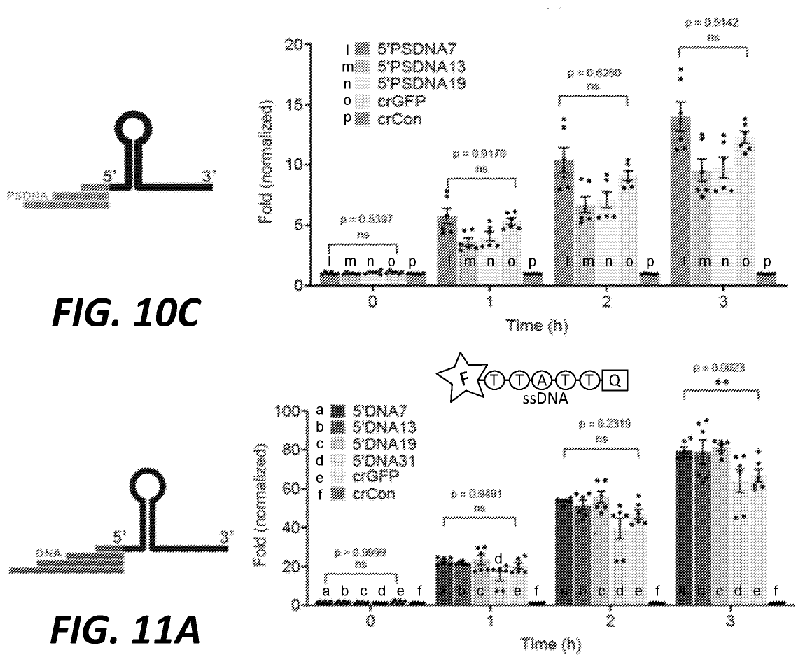

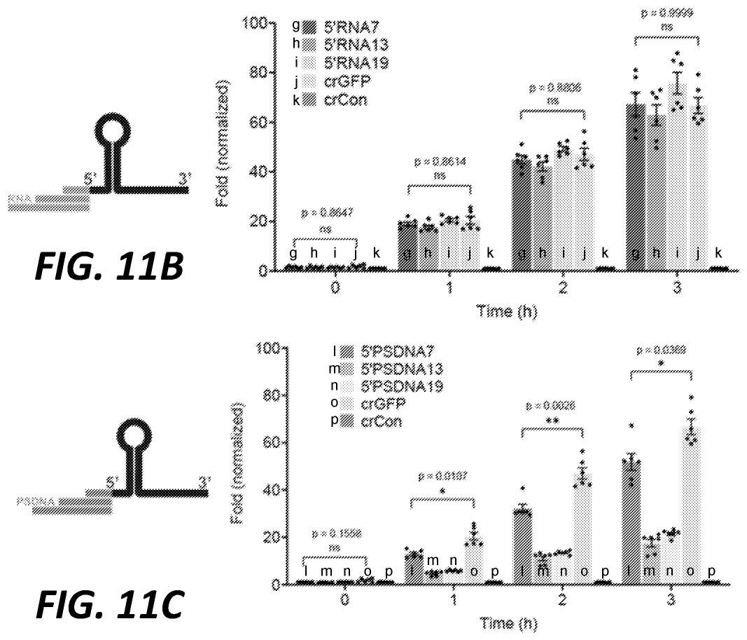

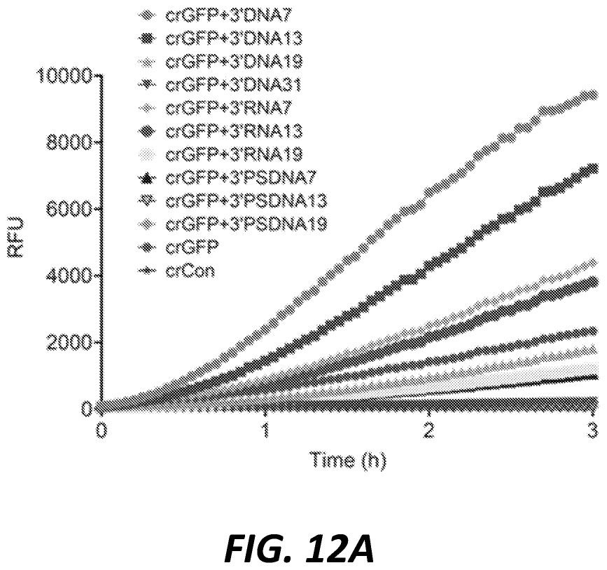

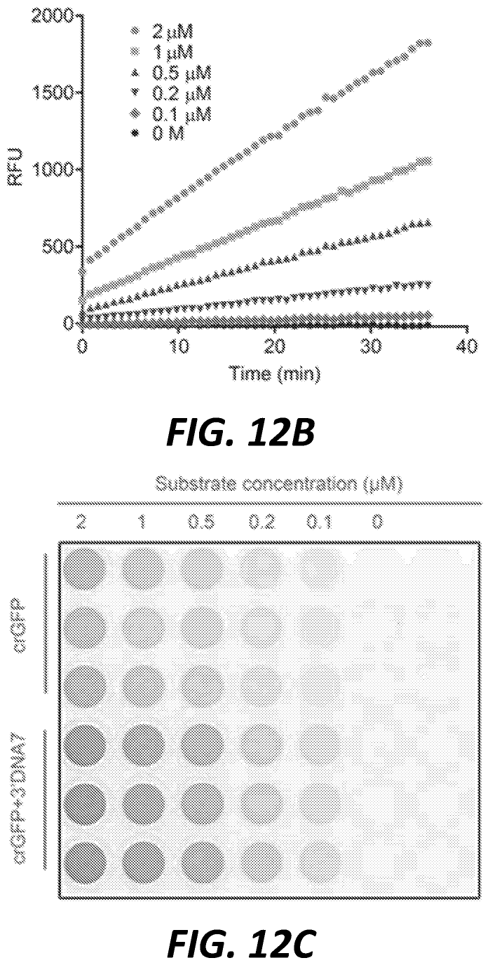

crRNA extensions affect Cas12a trans-cleavage activity. ssDNA, ssRNA, and phosphorothioate ssDNA extensions of various lengths ranging from 7 to 31 nucleotides were placed on either the 3′- or 5′-ends of the crRNA targeting GFP (green fluorescent protein), referred to here as crGFP (FIG. 1B-H). In order to measure the collateral or trans-cleavage activity of Cas12a, a FRET-based reporter used in DETECTR was employed¹, composed of a fluorophore (FAM) and a quencher (3IABkFQ) connected by a 5-nucleotide sequence (TTATT), which displays increased fluorescence upon cleavage. When using wild-type crRNAs, it was observed that the LbCas12a exhibited higher trans-cleavage activity than the AsCas12a or the FnCas12a. Using the same reporters, it was discovered that ssDNA and ssRNA extensions on the 3′-end of crGFP markedly enhanced the trans-cleavage ability of target-activated LbCas12a. Comparing the two types, the ssDNA extensions demonstrated higher activity than the corresponding ssRNA (FIGS. 1b-d,f and FIGS. 8A-12C). On the other hand, the phosphorothioate ssDNA extensions at the 3′- or 5′-end displayed minimal or no activity, showing decreased fluorescence intensity as modification length increased (FIGS. 1E,H and FIGS. 8A-12C).

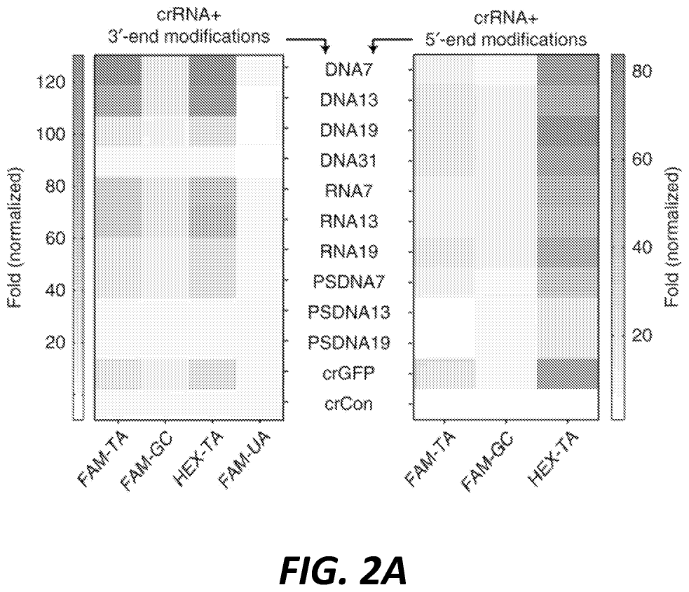

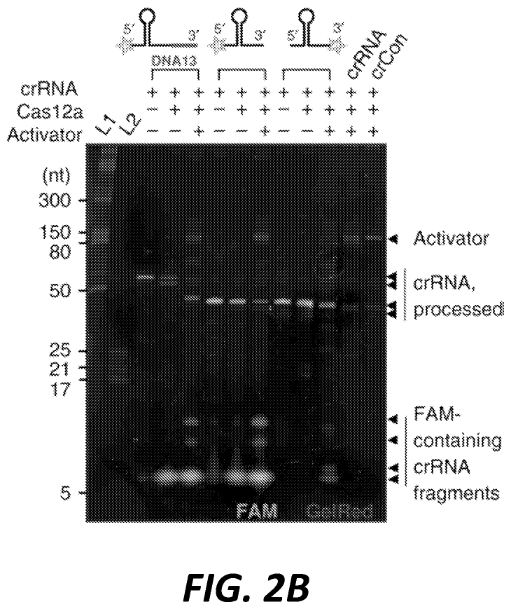

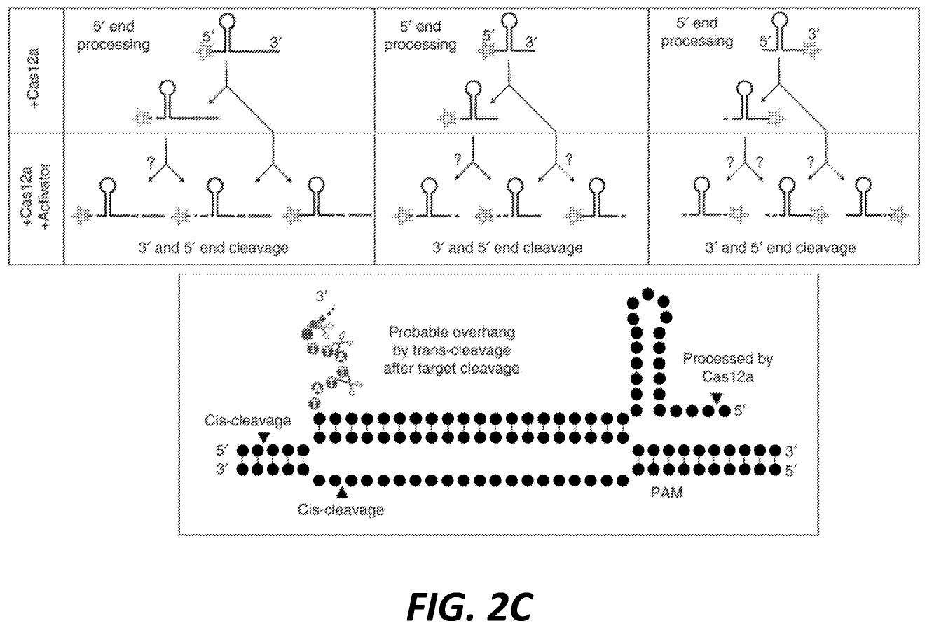

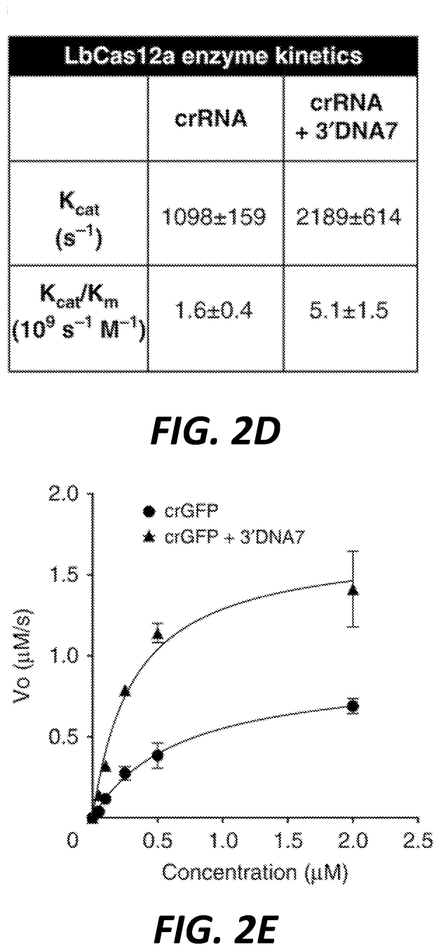

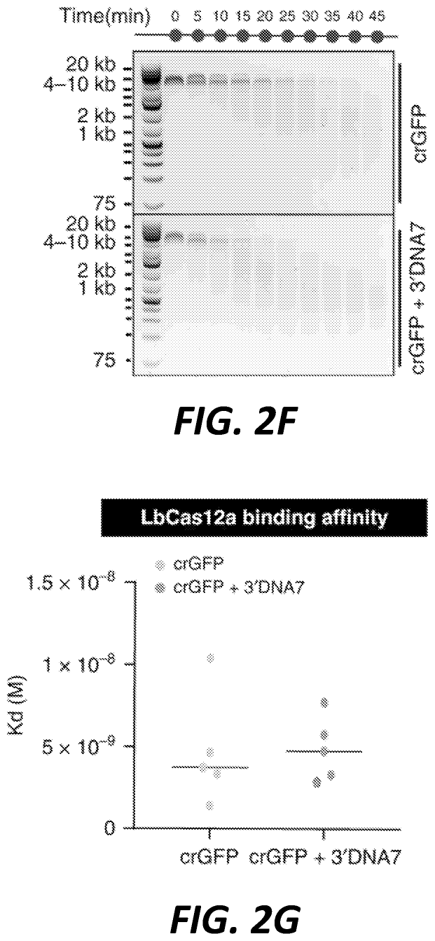

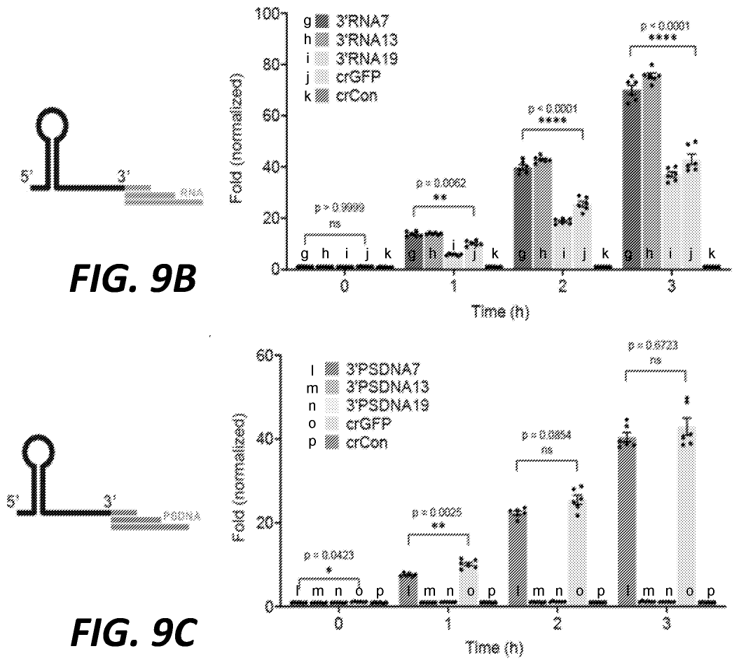

Notably, the 3′-DNA with 7-mer extensions on the crGFP, referred as crGFP+3′DNA7, yielded the highest fluorescence signal compared to other modifications, measuring approximately 3.5-fold higher intensity than the wild-type crGFP (FIG. 1C, FIGS. 8A, 9A). By investigating the conformational changes from the crystal structure of the binary LbCas12a:crRNA complex 12,15,16, it was observed that the 3′-end modifications on crRNA is proximal to the RuvC region of the LbCas12a. This supports the observation that the 3′-end extensions lead to higher trans-cleavage activity than the 5′-end. It is possible that the reporter composition itself may affect the LbCas12a collateral cleavage activity. Therefore, various nucleotides (GC and TA-rich) and fluorophores (FAM, HEX, and Cy5) within the reporter were tested. It was observed that the LbCas12a achieved maximal trans-cleavage activity with FAM or HEX and TA-rich reporter (FIG. 2A and FIGS. 8A-12C).

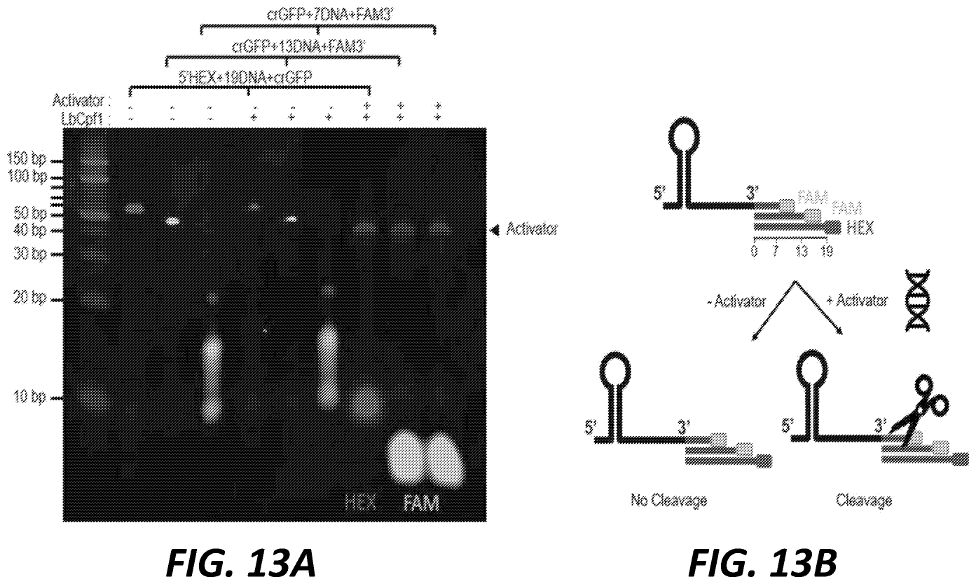

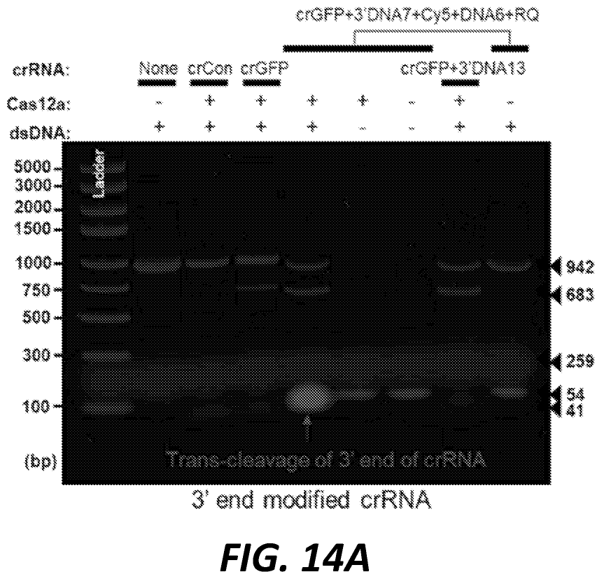

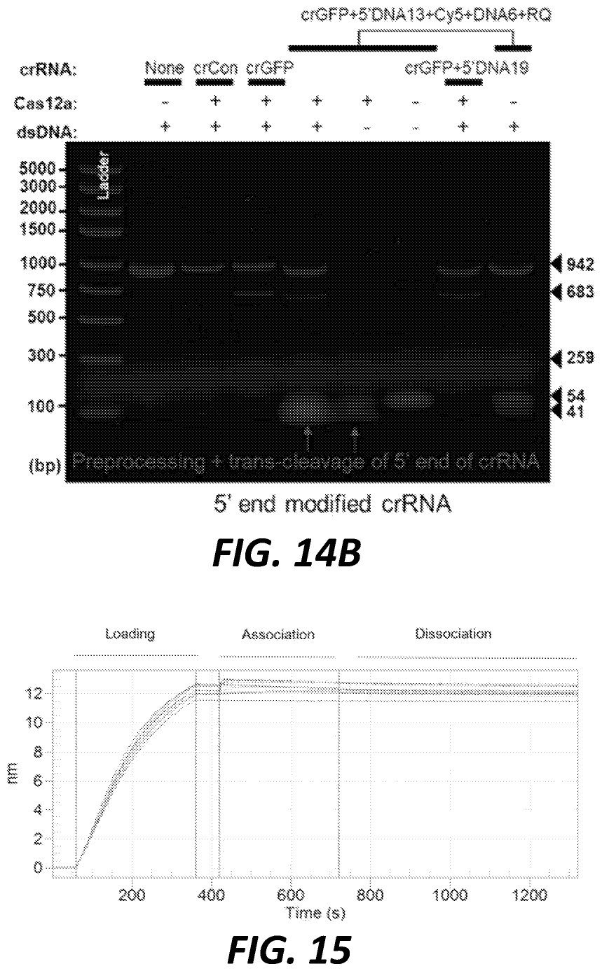

DNA-extended crRNA enhances the rate of trans-cleavage. It was speculated that once an R-loop is formed between crRNA and dsDNA or ssDNA activator, the LbCas12a executes a partial trans-cleavage of the 3′-end of crRNA, leaving an overhang. These remaining extensions may further expand the nuclease domain in the LbCas12a, resulting in conformational changes and allowing more access for nonspecific ssDNA cleavage. Different fluorophores, or fluorophore-quencher pairs separated by DNA linkers, were attached to either the 3′- or 5′-end of the crGFP with 7-mer DNA extensions and analyzed by denaturing gel electrophoresis. Surprisingly, it was discovered that the 3′-end of the crRNA is processed by LbCas12a only in the presence of an activator while the 5′-end is cleaved by LbCas12a even in the absence of the activator (FIG. 2B,C and FIGS. 13A-B,14A-B).

To further understand the LbCas12a enhanced enzymatic activity, a Michaelis-Menten kinetic study was performed on the wild-type crGFP and the crGFP+3′DNA7 was performed, and it was observed that the ratio Kcat/Km was 3.2-fold higher for crGFP+3′DNA7 than the unmodified crGFP (FIGS. 2D,E). The time-dependent gel electrophoresis analysis of nonspecific cleavage of ssDNA M13mp18 phage (˜7 kb) reconfirmed the fluorophore-quencher-based reporter assay results (FIG. 2F).

It was found that the trans-cleavage activity is drastically improved by 7-mer ssDNA extensions to the 3′-end of crGFP. It was studied whether the binding of crRNA with LbCas12a itself is influenced by such modifications. A biolayer interferometry binding kinetic assay revealed that the dissociation constant, K_{d}, between the binary complex LbCas12a:crRNA and LbCas12a:crRNA+3′DNA7 are comparable within a low nM scale (FIG. 2G and FIG. 15). These binding results suggest that the 3′DNA7 modification on crRNA does not affect the binary complex formation between the LbCas12a and the crRNA.

LbCas12a shows highest activity with TA-rich extended crRNAs. By placing the fluorophore FAM on the 5′-end and a 7-mer DNA extension on the 3′-end of the crGFP, it was found that the first Uracil on the 5′-end of the crGFP gets trimmed by LbCas12a in the absence of an activator, which corroborated previous studies reported for FnCas12a′² (FIG. 2B). It is possible that the 5′-end modifications are eliminated and converted back to the wild-type crRNA before complexing with the activator. This finding reinforces the observation that the 5′-extended crRNA has similar collateral cleavage activity as the wild-type crRNA.

### Example 3. The Modified Pre-crRNA and Modified Mature crRNA (Tru-crRNA) Exhibited Comparable Trans-Cleavage Efficiency

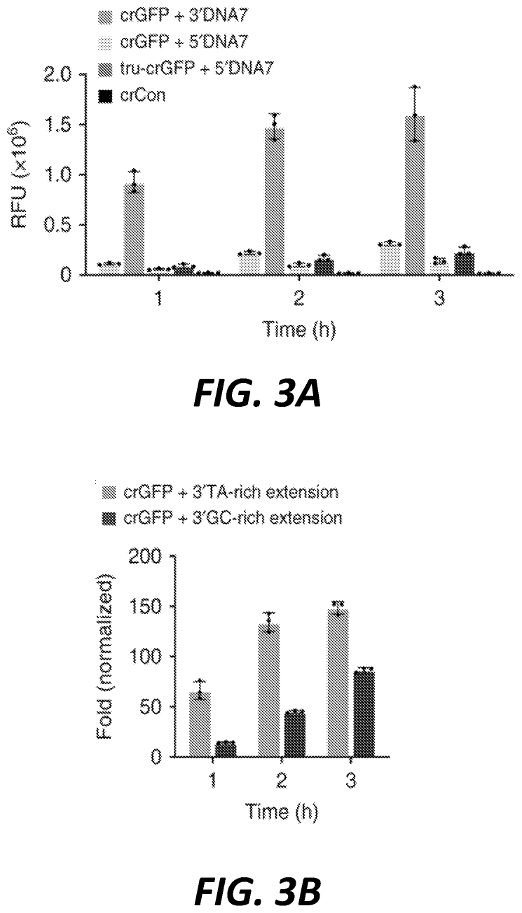

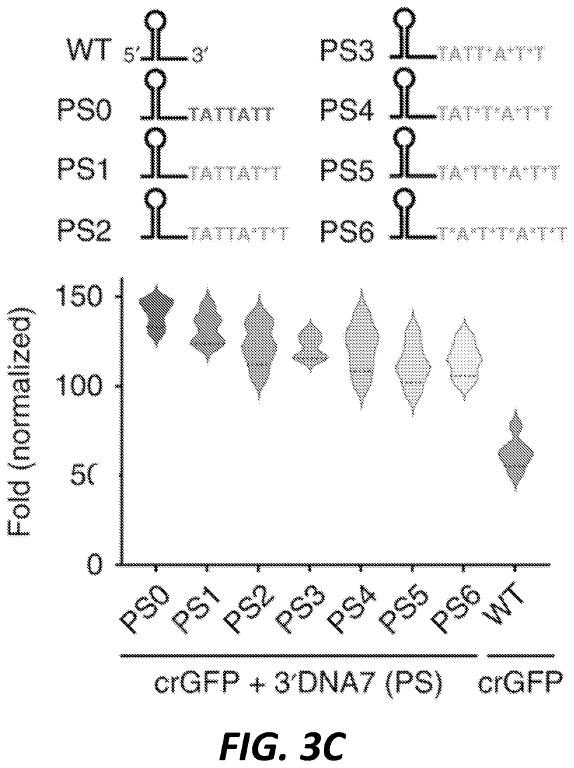

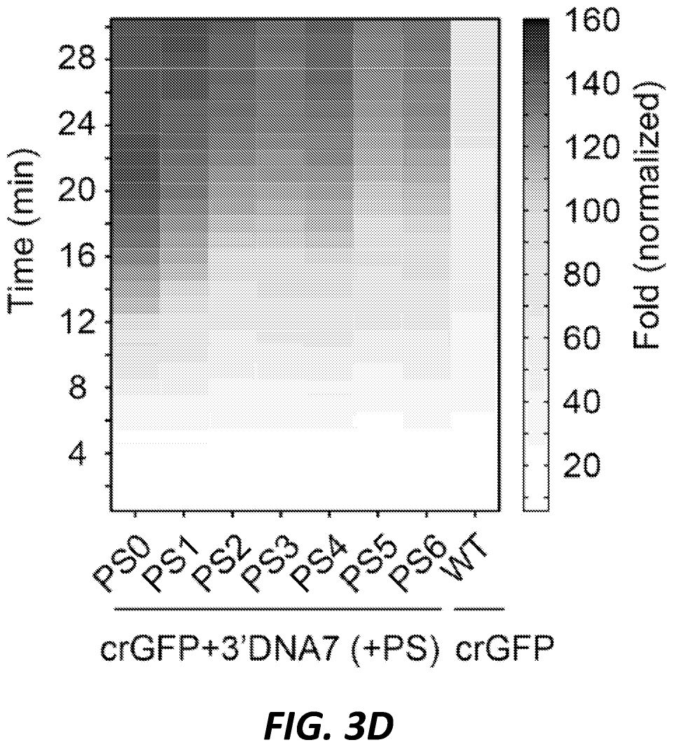

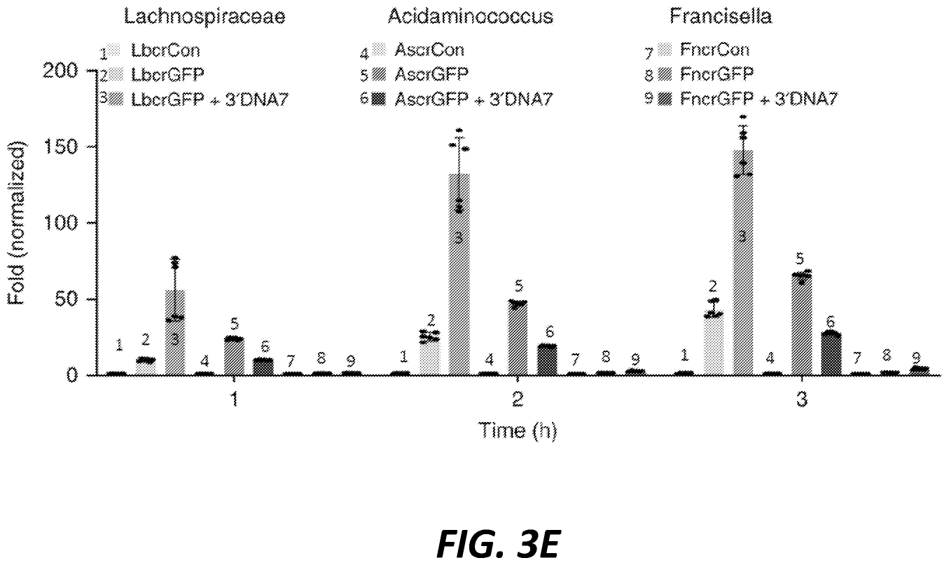

How extensions of the mature crRNA would influence the trans-cleavage activity compared to the corresponding extended pre-crRNA was investigated. It was discovered that the modified pre-crRNA and modified mature crRNA (tru-crRNA) exhibited comparable trans-cleavage efficiency (FIG. 3A). Furthermore, when a dsDNA or an ssDNA activator was present, the 3′- and 5′-end DNA-extended crRNA were cleaved (FIGS. 2B,C and FIGS. 13A-B, 14A-B).

### Example 4. The crRNA with TA-Rich Extensions Carried Out More Trans-Cleavage than Those with GC-Rich Regions

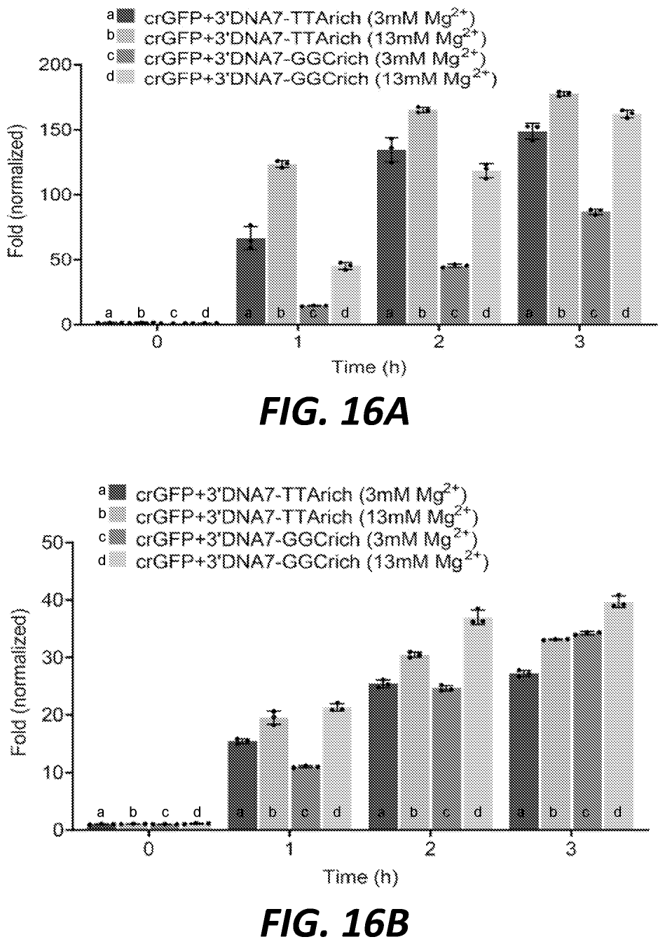

The nucleotide content of the extended regions of the crGFP was altered to test whether the trans-cleavage activity is dependent on the sequence of ssDNA extensions on 3′-end of the crRNA. It turned out that the crGFP with TA-rich extensions carried out significantly more collateral cleavage than those with GC-rich regions (FIG. 3B and FIG. 16A-B).

### Example 5. The LbCas12a Trans-Cleavage Activity Decreased as More Phosphorothioate Modifications were Added to the Extension

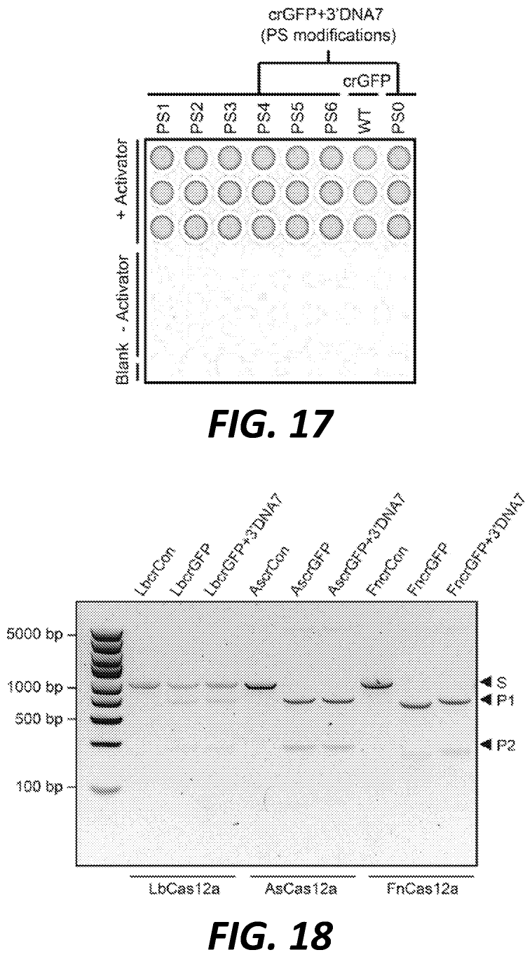

Non-fully phosphorothioate of the crGFP+3′DNA7 with 1 to 6 PS substitutions starting from the 3′-end of the extension inwards was studied. Whether the trans-cleavage activity of LbCas12a could be enhanced further by protecting the DNA extension with phosphorothioate modifications was studied. Interestingly, fluorescence-based reporter assays showed that the LbCas12a trans-cleavage activity decreased as more phosphorothioate modifications were added to the extension, with the non-phosphorothioated crRNA+3′DNA7 exhibiting highest fluorescence signal (FIG. 3C,D and FIG. 17).

### Example 6. LbCas12a Showed the Higher Fluorescence Signal than AsCas12a and FnCas12a

Other orthologs of Cas12a were studied. An in vitro cis-cleavage and trans-cleavage assay of AsCas12a and FnCas12a with an extended crGFP was compared to a wild-type crGFP (FIG. 3E and FIG. 18). Interestingly, the crGFP+3′DNA7 showed similar results with FnCas12a; however, it exhibited an opposite effect with AsCas12a. However, the cis-cleavage activity was found to be comparable between the crGFP and crGFP+3′DNA7 for all the orthologs tested. Overall, LbCas12a showed the highest fluorescence signal, which is consistent with previous studies^{13,18}. Through observation of the fluorophore-quencher-based reporter assay and time-dependent gel electrophoresis, we hypothesized that the various extensions of ssDNA on the crRNA induce conformational changes on LbCas12a that result in enhanced endonuclease activity.

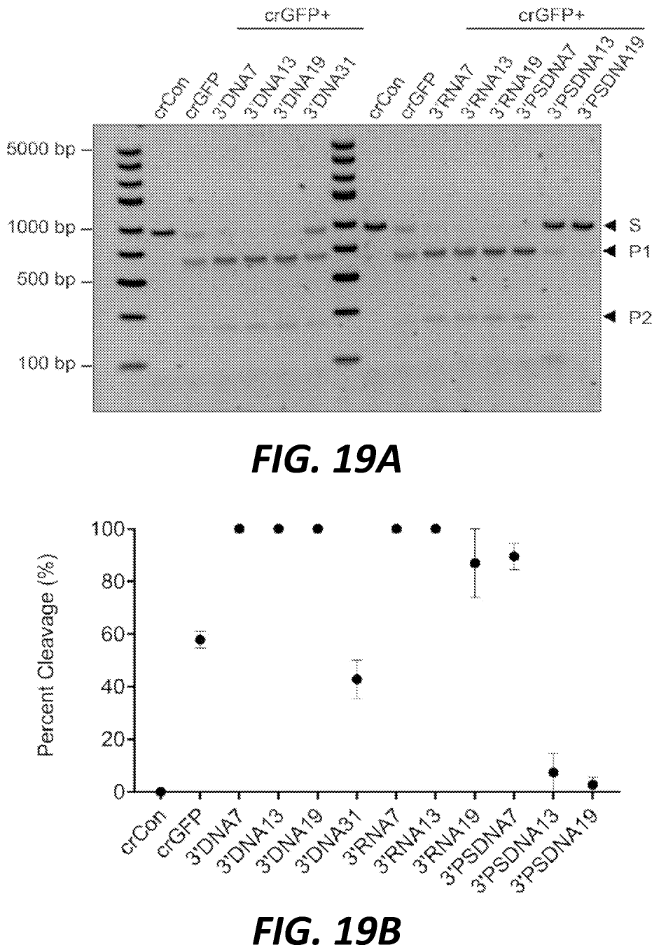

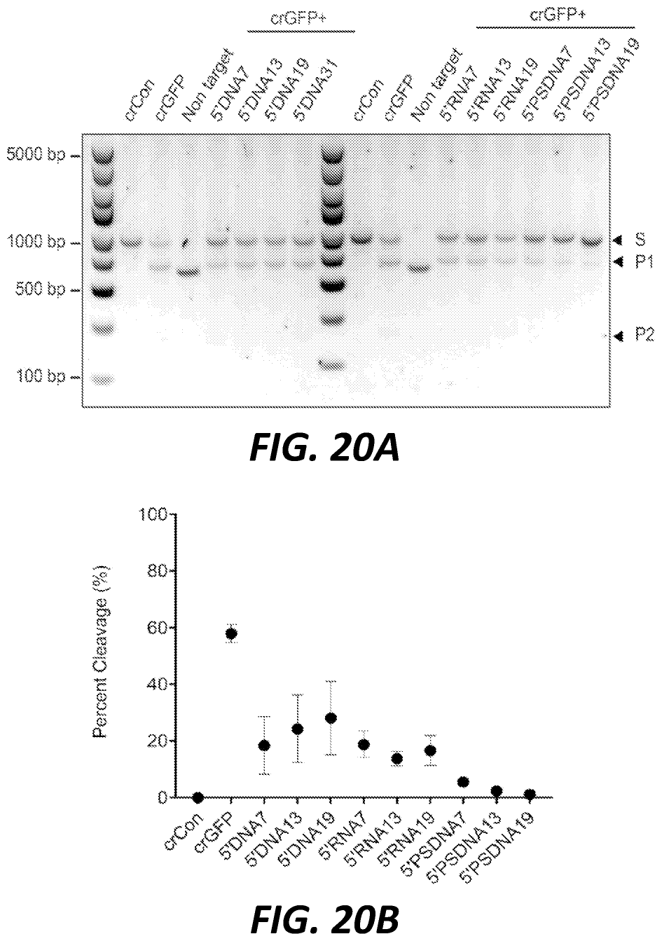

Structural analysis of LbCas12a shows that it contains a single RuvC domain, which processes precursor crRNA into mature crRNA, cleaves target dsDNA or ssDNA (referred here as activators), and executes nonspecific cleavage afterwards^{19,20}. The effects of these modified crRNAs on cis-cleavage compared to the wild-type crRNA, as well as how cis-cleavage activity correlates to the trans-cleavage activity, were investigated. An in vitro cis-cleavage assay for various 3′-end and 5′-end modifications was performed. It was noticed that the cis-cleavage activity was either similar or marginally improved with most 3′-end modifications while the 5′-end modifications showed either similar or slightly reduced activity. This phenomenon suggests that the trans-cleavage activity is commensurate with the cis-cleavage activity (FIGS. 19A-20B).

### Example 7. The crRNA+3′DNA7 Enhances the Specificity of Detection when Compared to crRNA-WT

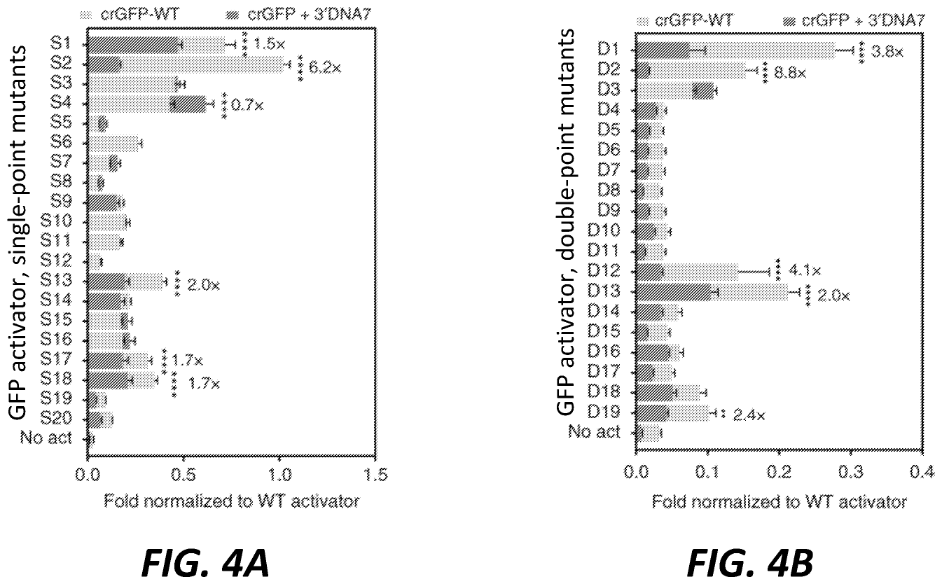

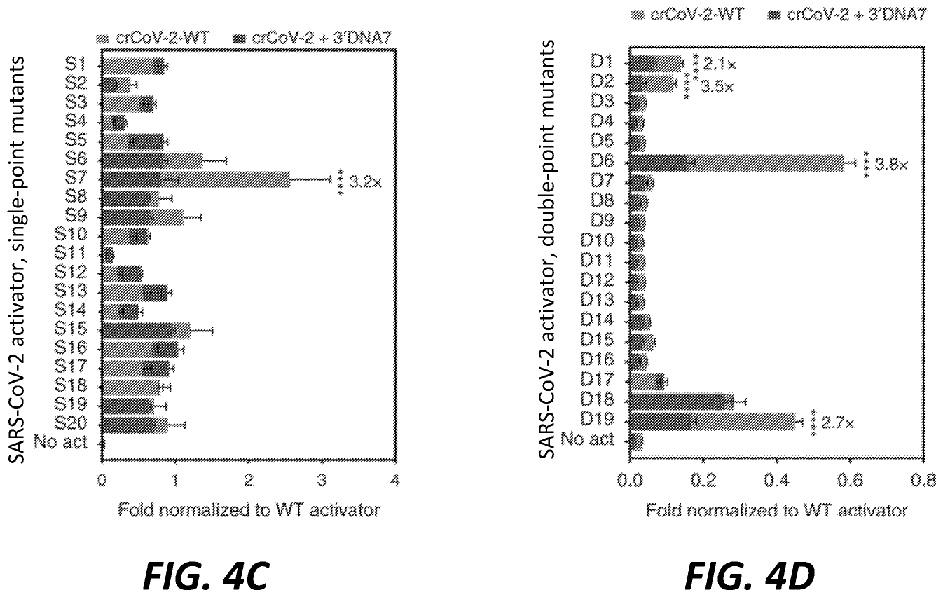

ENHANCE improves specificity of target detection. The specificity of these extended crRNAs was investigated in discriminating point mutations across dsDNA. By mutating either a single nucleotide or two consecutive nucleotides at each position across the target-binding region, it was observed that the crRNA+3′DNA7 tolerated mutations and produced a stronger fluorescence signal than the wild-type crRNA for both GFP and SARS-CoV-2 targets. Single point mutants were more easily tolerated than double mutants by LbCas12a. Nevertheless, it was exciting to note that the fluorescence intensity ratio or the fold-change normalized to the wild-type dsDNA targets was significantly lower for the crRNA+3′DNA7 compared to wild-type crRNA (FIG. 4A-D) across both the genes tested. It was observed that the 3′DNA7 modifications on crRNAs enhance specificity by up to 8.8-fold across various off-targets when compared to crRNA-WT. Furthermore, based on the statistical analysis, crRNA+3′DNA7 did not significantly reduce the specificity of detection for the tested targets.

### Example 8. Divalent Cations are Crucial for ENHANCE

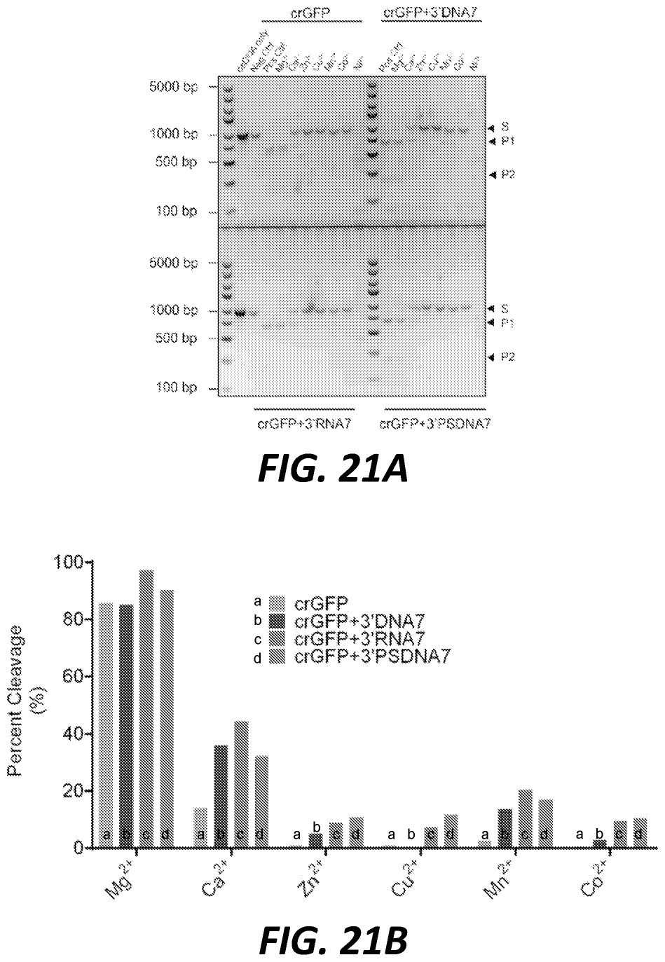

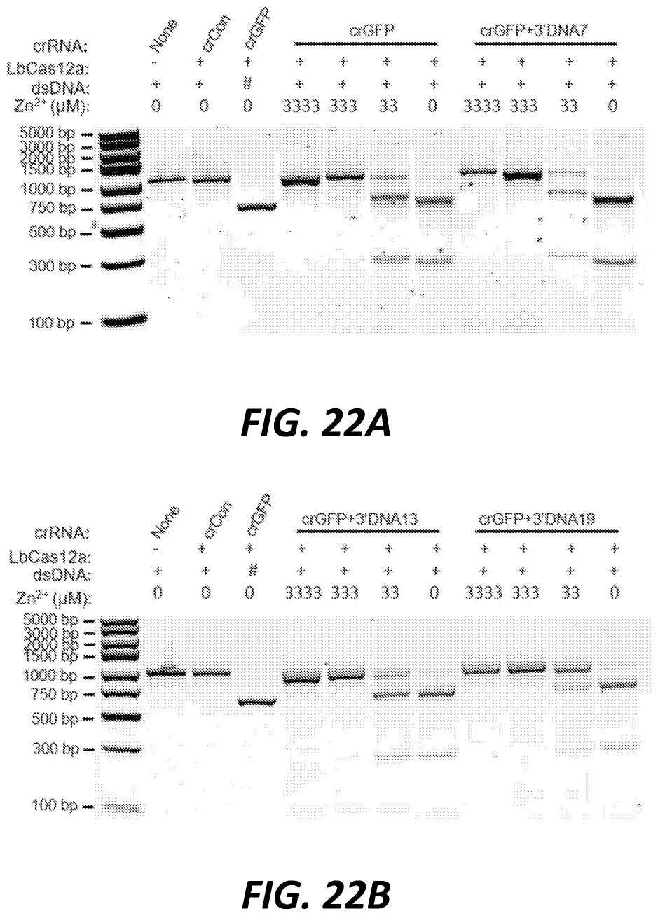

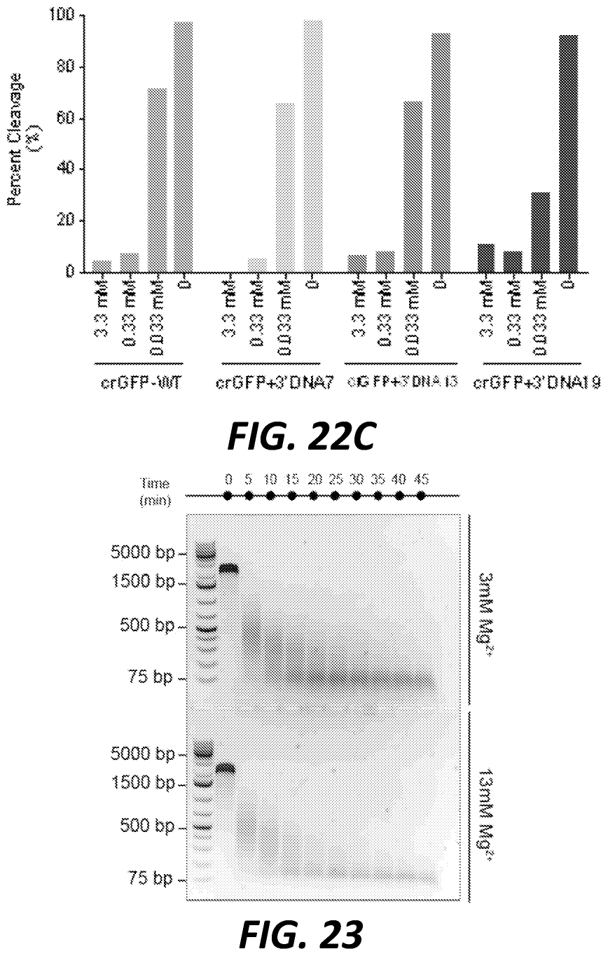

FnCas12a is a metal-dependent endonuclease, and magnesium ions are required for FnCas12a-mediated self-processing of precursor crRNA. Different metal ions may significantly affect the trans-cleavage activity of LbCas12a. A range of divalent metal cations was tested, and it was discovered that most ions including Ca²⁺, Co²⁺, Zn²⁺, Cu²⁺, and Mn²⁺ significantly inhibited the LbCas12a activity (FIG. 21A-B). The Zn²⁺ mediated inhibition of LbCas12a was investigated, it was found that the inhibition was dose-dependent (FIG. 22A-B). Interestingly, Ni²⁺ ions showed an unusual cis-cleavage activity possibly due to its interactions with the His tags on LbCas12a (FIG. 21A-B).

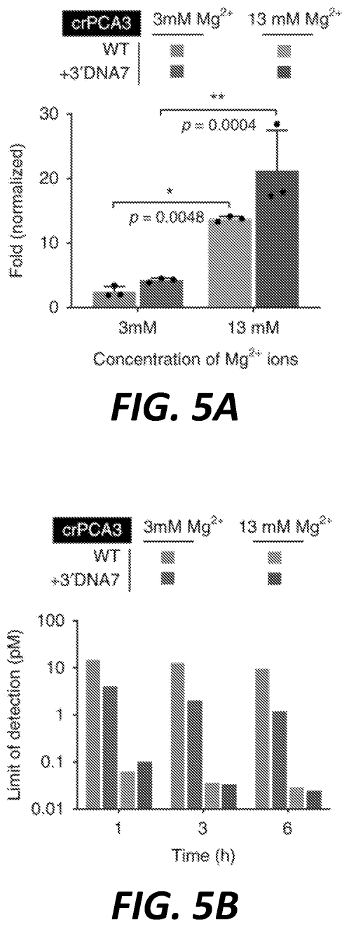

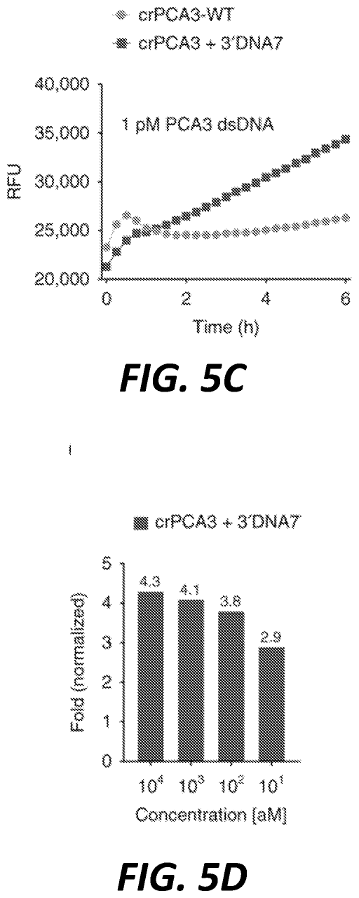

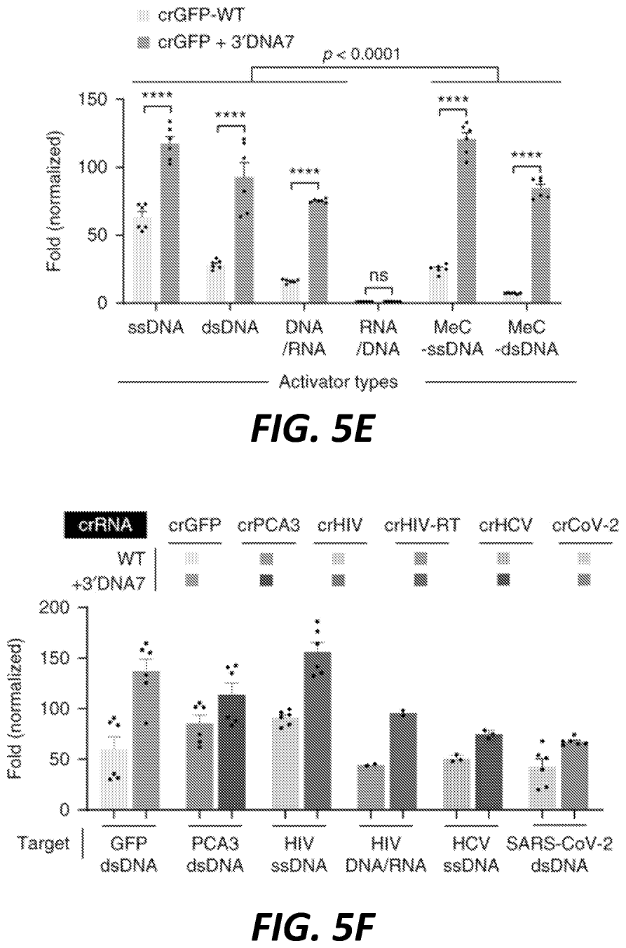

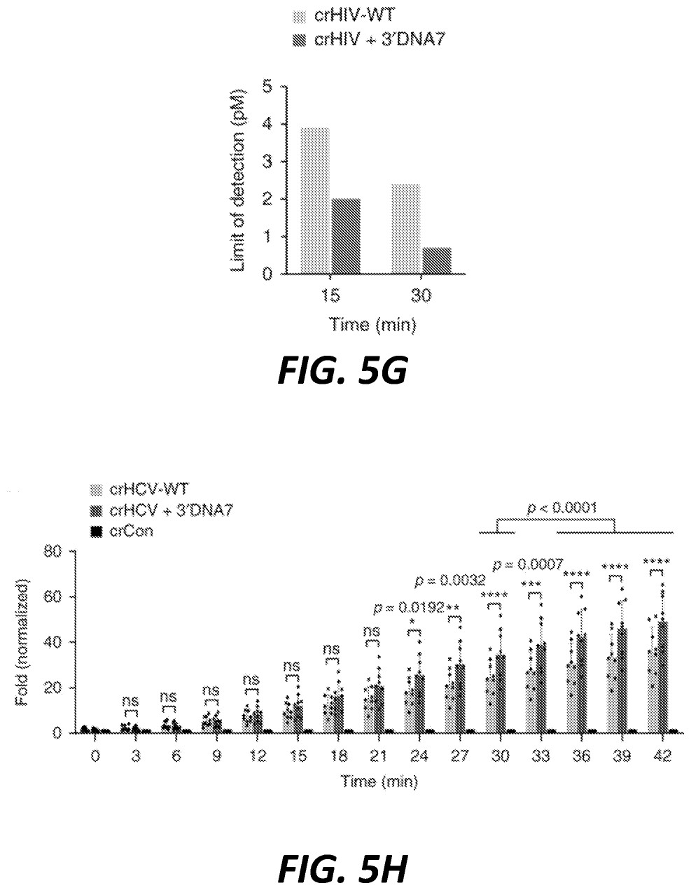

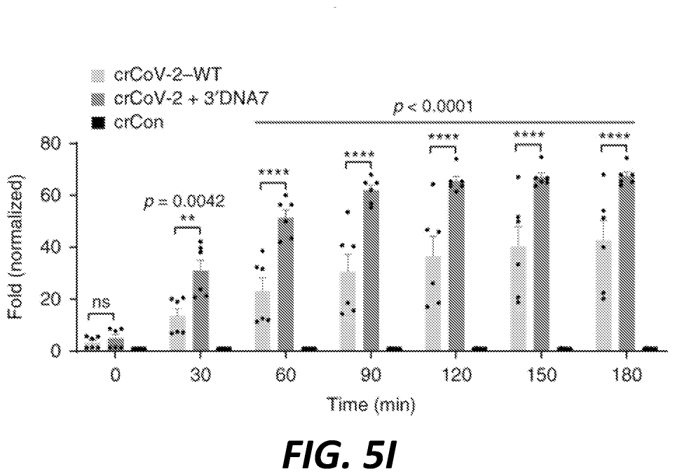

Among the tested divalent metal ions, the Mg²⁺ ions showed the highest in vitro cis-cleavage activity. Therefore, the effect of Mg²⁺ ions on trans-cleavage activity of LbCas12a was investigated. With increasing the concentration of Mg²⁺ ions, a significant increase in fluorescence signal was observed in an in vitro trans-cleavage assay. By varying the amount of Mg²⁺ in the Cas12a reaction, it was identified that the optimal condition of Mg²⁺ was around 13 mM (FIG. 5a-b and FIGS. 23-26).

ENHANCE works robustly towards a broad range of targets. To validate the CRISPR-ENHANCE technology, a clinically relevant nucleic acid biomarker, Prostate Cancer Antigen 3 (PCA3/DD3), was first selected, which is one of the most overexpressed genes in prostate cancer tissue and excreted in patients' urine. Consequently, elevated PCA3 levels during prostate cancer progression has become a widely targeted biomarker for detection²¹⁻²⁴. To determine the limit of detection of PCA3 using our ENHANCE technology, the PCA3 cDNA into synthetic urine was spiked, and how this clinically relevant environment affects the activity of Cas12a was investigated.

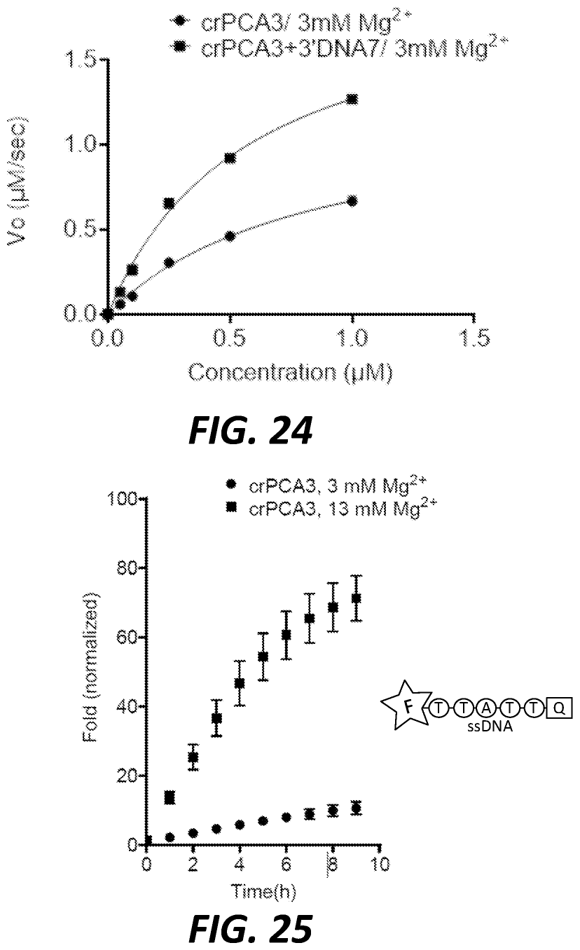

Using ENHANCE for detecting the PCA3 cDNA, the limit of detection was determined to be as low as 25 fM in the urine at 13 mM Mg²⁺ concentration compared to ˜1 pM at 3 mM Mg²⁺ concentration after 6 hours (FIGS. 5A-C and FIGS. 24-26). In contrast, the wild-type crRNA also showed a similar 29 fM limit of detection at 13 mM Mg²⁺ concentration while the limit of detection was ˜10 pM at 3 mM Mg²⁺ concentrations after 6 hours. Therefore, by combining the crRNA modifications with increased Mg²⁺ ion concentrations, approximately 400-fold increase in sensitivity was achieved, based on limit of detection calculations. Nevertheless, this observation also suggests that the modified crRNA+3′DNA7 significantly improves the limit of detection at low Mg²⁺ but reaches a saturation point that is comparable with the wild-type crRNA at high Mg²⁺ concentration. To understand the importance of divalent ions in the Cas12a trans-cleavage reaction, a Michaelis-Menten kinetic study with various Mg²⁺ concentrations was carried out (FIG. 24). It was observed that the initial reaction rate of Cas12a in the presence of high Mg²⁺ concentrations increased tremendously compared to that in low Mg²⁺.

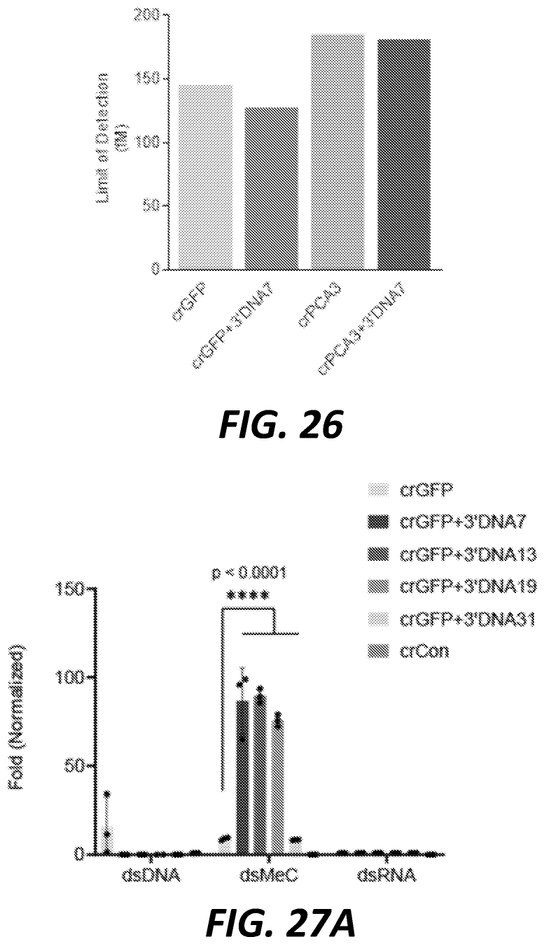

However, the two reaction rates eventually reach a similar saturation point (FIGS. 23-25). This suggests that Mg²⁺ is not only required for the Cas12a reaction, but also accelerates the enzyme's trans-cleavage activity. Regardless, Mg²⁺ plays an important role in lowering the limit of detection in synthetic urine containing PCA3. While as low as 25 fM (equivalent to 2.5 attomoles) of PCA3 cDNA can be detected with ENHANCE without any target amplification (FIG. 26), the clinical concentration of PCA3 mRNA in the urine can be lower and therefore may require target pre-amplification^{25,26}. A recombinase polymerase amplification (RPA) step was used to isothermally amplify the PCA3 cDNA. By combining the RPA step as previously reported^{1,7}, the concentration of PCA3 cDNA in the urine was detectable down to ˜10 aM (1 zmol) with 2.9-fold signal to noise ratio (FIG. 5D).

### Example 9. The crRNA+3′DNA7 Possesses Enhanced Trans-Cleavage Activity in Detecting Methylated DNA Compared to crRNA-WT

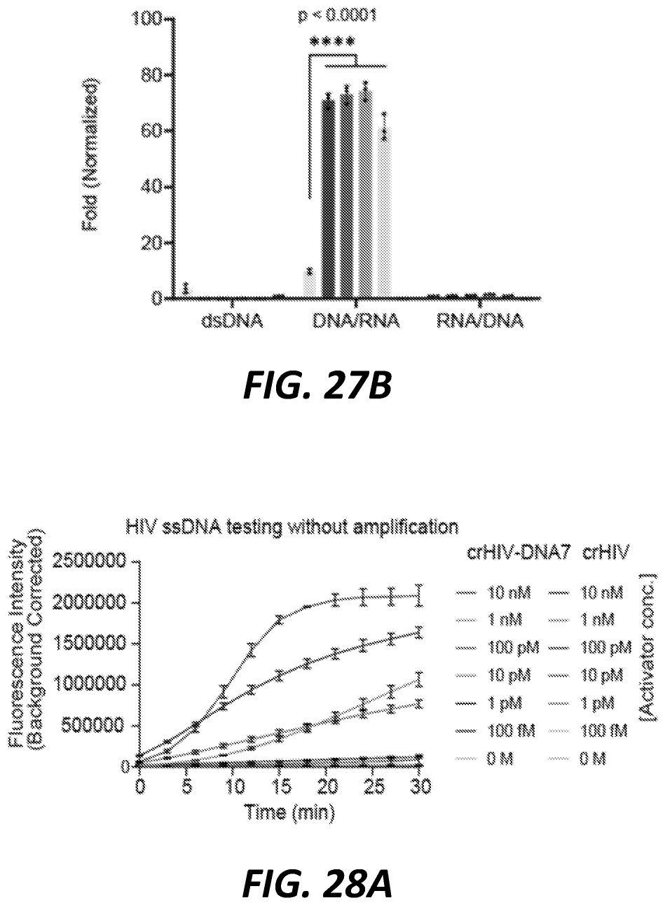

While crRNA/LbCas12a has been traditionally used to detect unmodified DNA, the field is missing the knowledge on how the common epigenetic marker, DNA methylation, affects its trans-cleavage activity. DNA methylation is also one of the bacterial defense systems that fight against outside invaders. It would be fascinating to understand how LbCas12a collateral cleavage is able to recognize methylated DNA targets. It was discovered that the wild-type crRNA had significantly reduced activity in detecting methylated DNA, containing 5-methyl cytosine, compared to the unmethylated DNA. However, the ENHANCE showed 5.4-fold and 3.4-fold and higher trans-cleavage activity compared to the wild-type crRNA for targeting the methylated dsDNA and ssDNA, respectively (FIG. 5E and FIG. 27A).

### Example 10. The crRNA+3′DNA7 Possesses Enhanced Sensitivity in Detecting a Target DNA in ssDNA, dsDNA, or DNA/RNA Heteroduplex

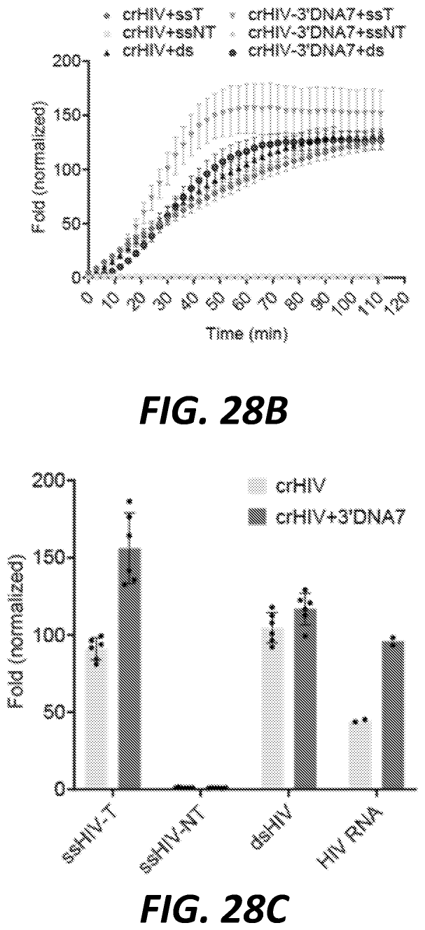

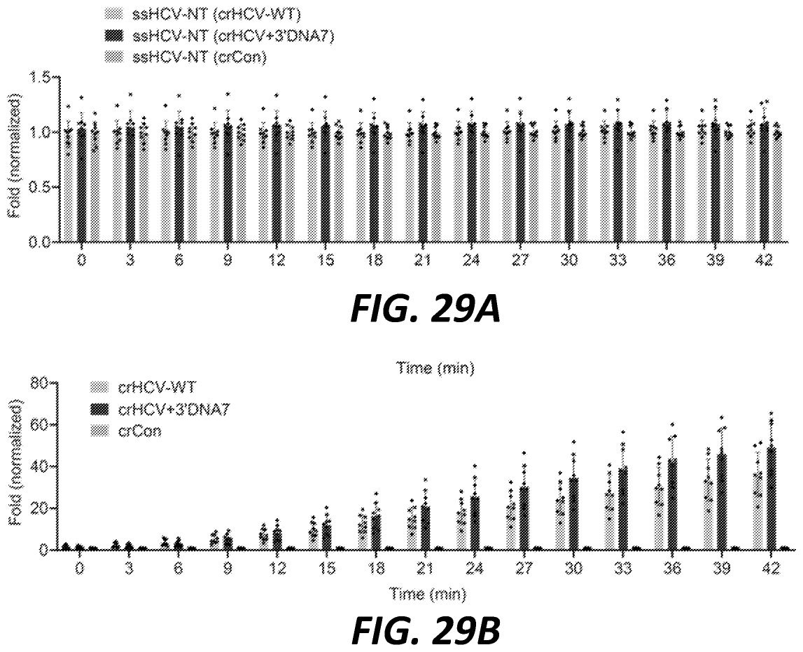

A reverse transcription step to convert RNA into cDNA/RNA heteroduplex was incorporated before detecting the RNA with a trans-cleavage assay. It was discovered that the RNA can only be detected if the target strand for crRNA is a DNA but not an RNA in a heteroduplex. Notably, the efficiency of the trans-cleavage activity for the DNA/RNA heteroduplex was found to be significantly lower than the corresponding ssDNA or dsDNA (FIG. 5E, FIG. 27B). However, the DNA/RNA heteroduplex achieved an improved enzymatic collateral activity when using the crRNA+3′DNA7 compared to the wild-type crRNA. The ENHANCE was used to successfully detect low picomolar concentrations of HIV RNA target encoding Tat gene with our DNA/RNA heteroduplex detection strategy (FIG. 5F). In parallel, ssDNA and dsDNA targets from HIV were also detected with much higher sensitivity compared to the wild-type crRNA within 15 to 30 minutes (FIGS. 5F,G and FIGS. 28A-C). The ENHANCE was further used for detecting HCV ssDNA and HCV dsDNA gene encoding a polyprotein precursor, both of which indicated consistent enhanced collateral activity than the wild-type crRNAs within 24 minutes (FIGS. 5F-H and FIG. 29). The limit of detection for HIV and HCV targets were calculated to be 700 fM cDNA and 290 fM ssDNA, respectively.

### Example 11. The crRNA+3′DNA7 Possesses Enhanced Sensitivity and Specificity in Detecting SARS-CoV-2 Coronavirus

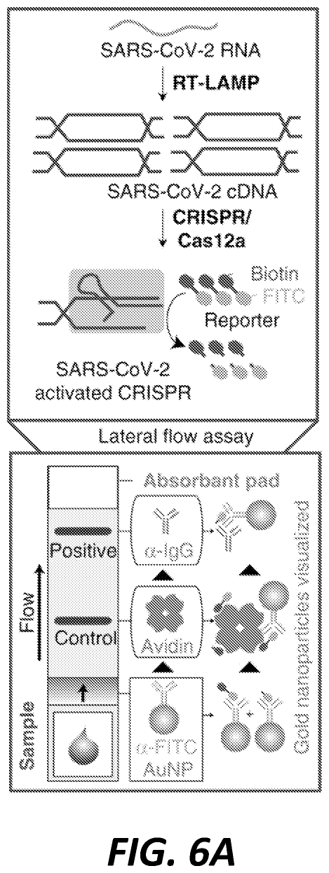

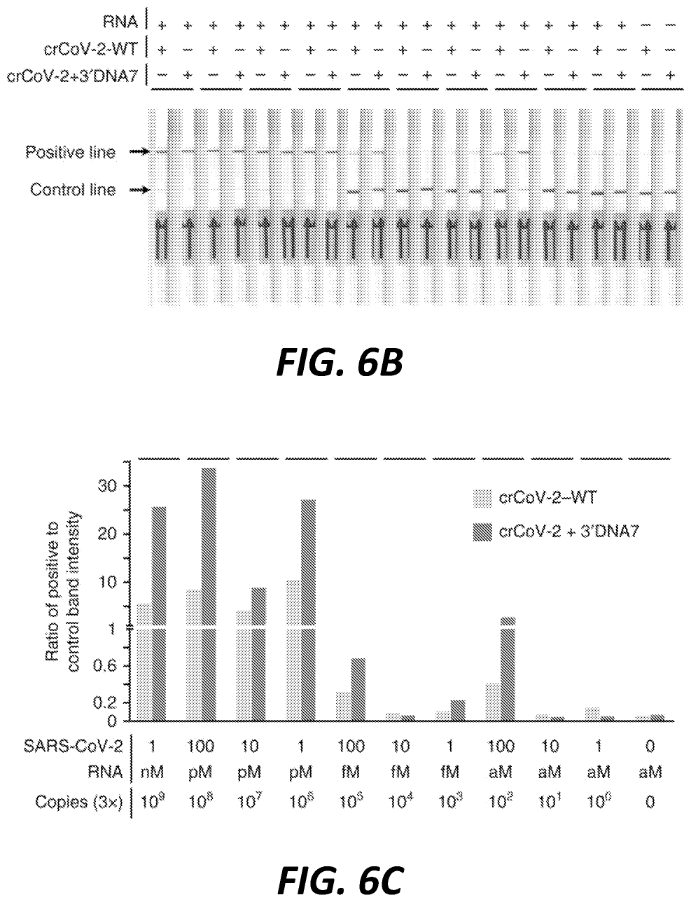

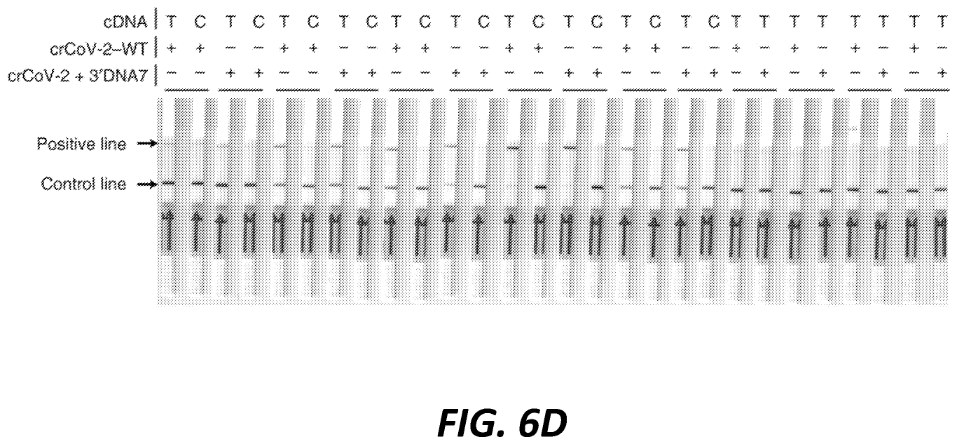

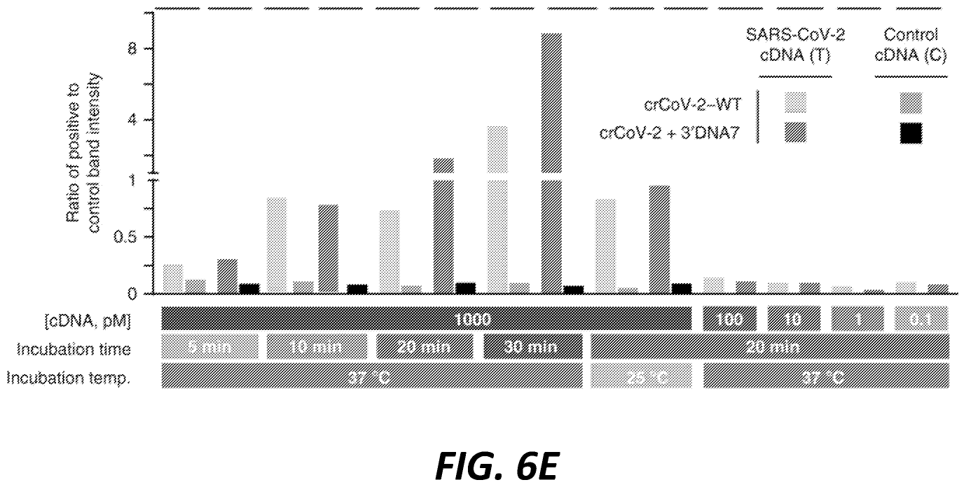

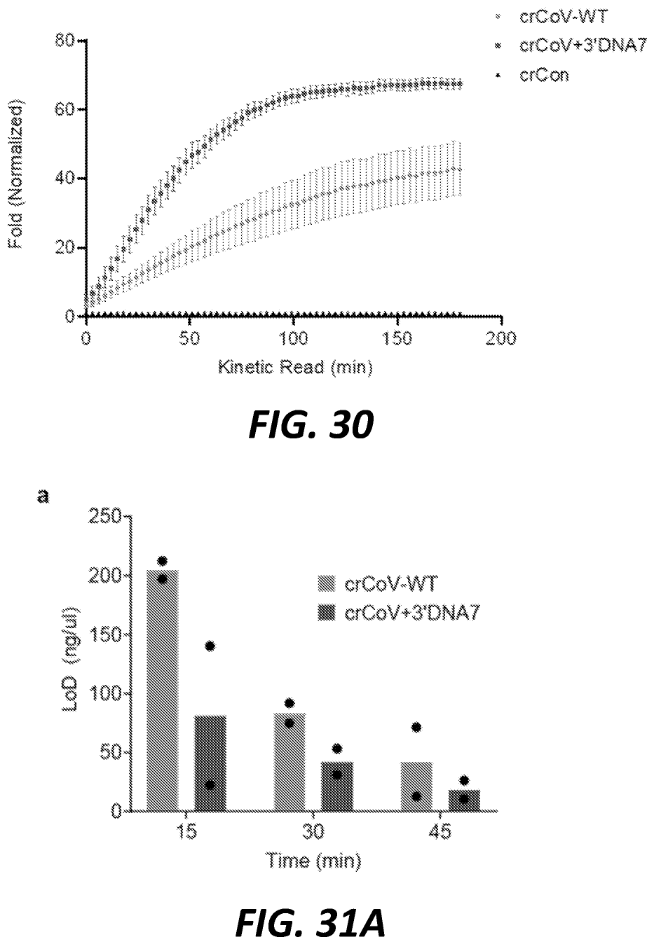

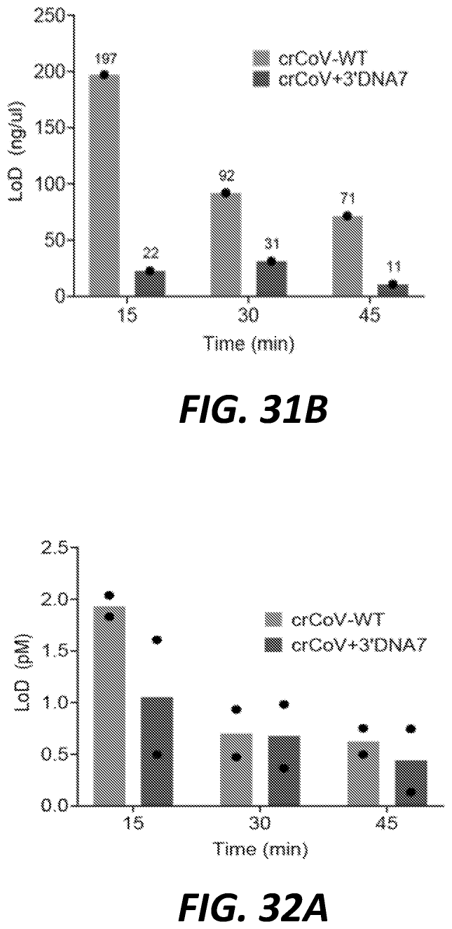

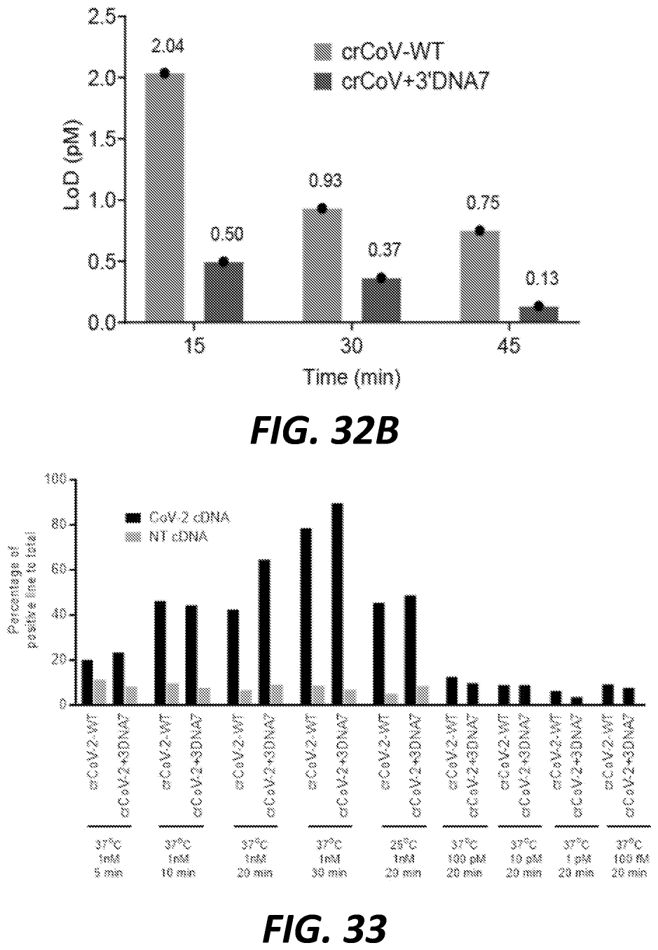

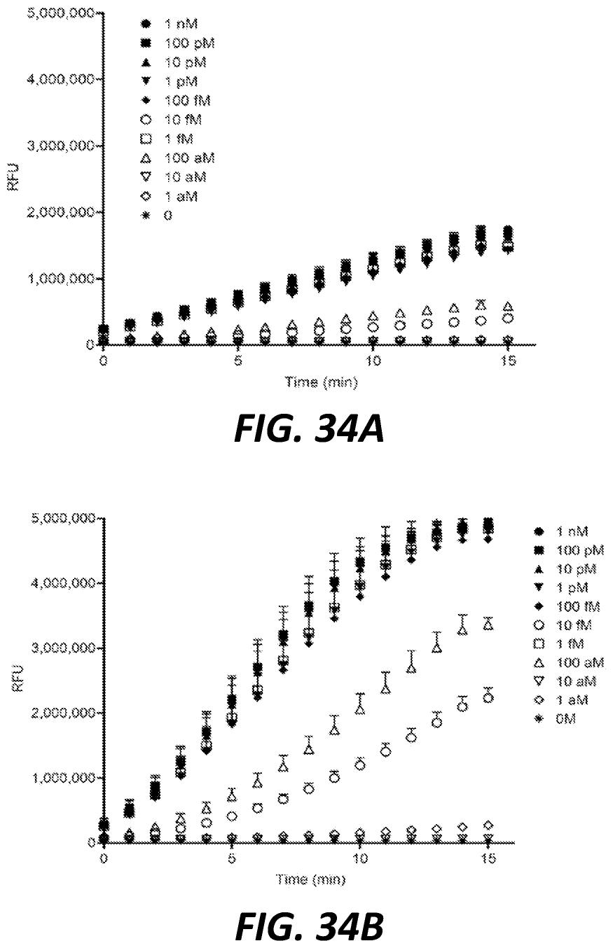

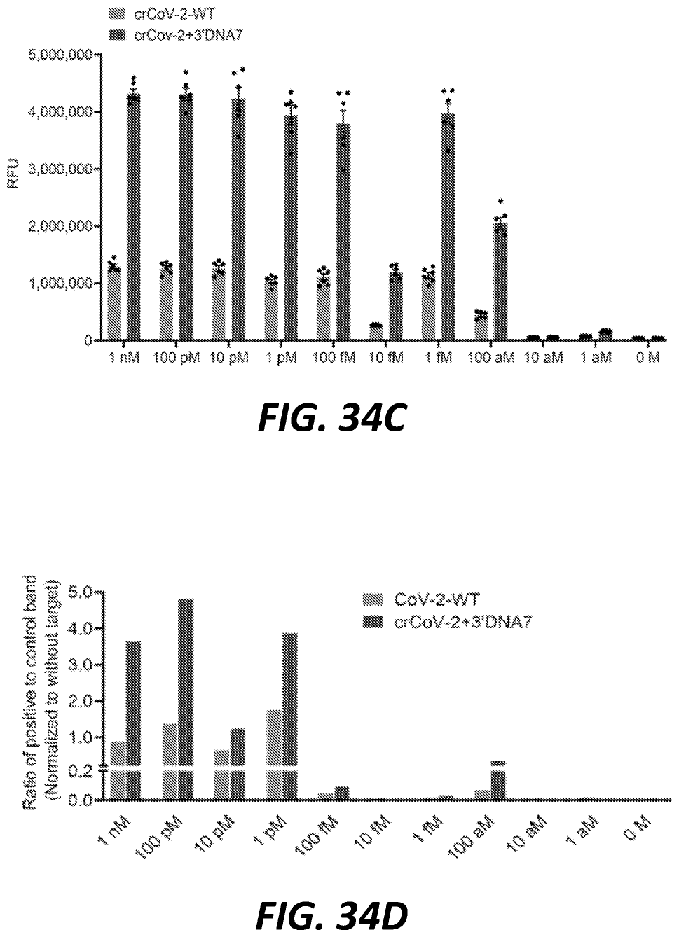

ENHANCE detects SARS-CoV-2 genomic RNA with high sensitivity. In the wake of the recent COVID-19 pandemic, there is an urgent need to rapidly detect the SARS-CoV-2 coronavirus (referred as CoV-2 here for simplicity). The ENHANCE was used to detect CoV-2 dsDNA by designing crRNAs targeting nucleocapsid phosphoprotein encoding N gene (FIGS. 3f,i). While no clinical samples were tested, the results indicated the 3′DNA7-modified crRNA consistently demonstrated higher sensitivity for detecting CoV-2 dsDNA within 30 minutes as compared to the wild-type crCoV-2 (FIGS. 30-32). By incorporating a commercially available paper-based lateral flow assay with a FITC-ssDNA-Biotin reporter^{1,27,28}, 1 nM of CoV-2 cDNA could be visually detected at room 25° C. within 20 minutes of incubation using both wild-type and modified crRNAs without any target amplification (FIG. 6 and FIG. 33). The enzyme trans-cleavage activity exhibited a consistent trend with the crRNA+3′DNA7 among five different targets (FIG. 5F). When incorporating a reverse transcription step and a loop-mediated isothermal amplification (RT-LAMP) strategy into the ENHANCE, both the crCoV-2-WT and the crCoV-2+3′DNA7 demonstrated a limit of detection down to a 3-300 copies of RNA (FIGS. 6A-C). However, in case of crCoV-2-WT, the partial cleavage of the reporter resulted in a darker control line on the paper strip. Band-intensity analysis showed that the ENHANCE exhibited an average of 23-fold higher ratio of positive to control line between 1 nM (3×10⁹ total copies) and 1 pM (3×10⁶ total copies) of target CoV-2 RNA, while the crCoV-2-WT indicated an average of only 7-fold ratio (FIGS. 6A-C and FIG. 34A-D).

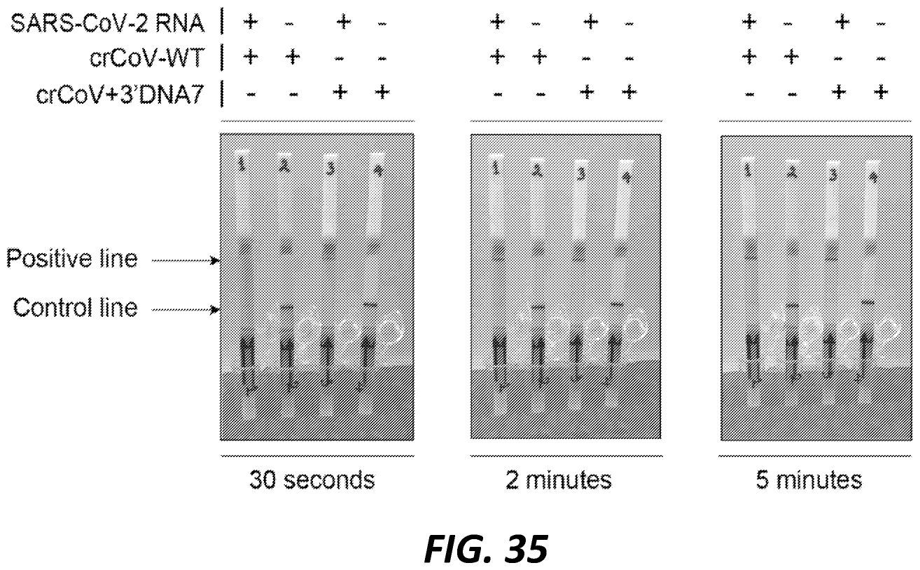

A much higher fluorescence intensity was observed when using ENHANCE than the unmodified CRISPR in a very short amount of time, within 10 minutes, for detecting targets. When the system was applied on a lateral flow assay, the positive band is visible only after 30 seconds whereas it takes over 1 minute to show up when using the unmodified crRNA. The engineered ENHANCE system may be used for a much rapid detection of nucleic acids including SARS-CoV-2 (FIG. 35).

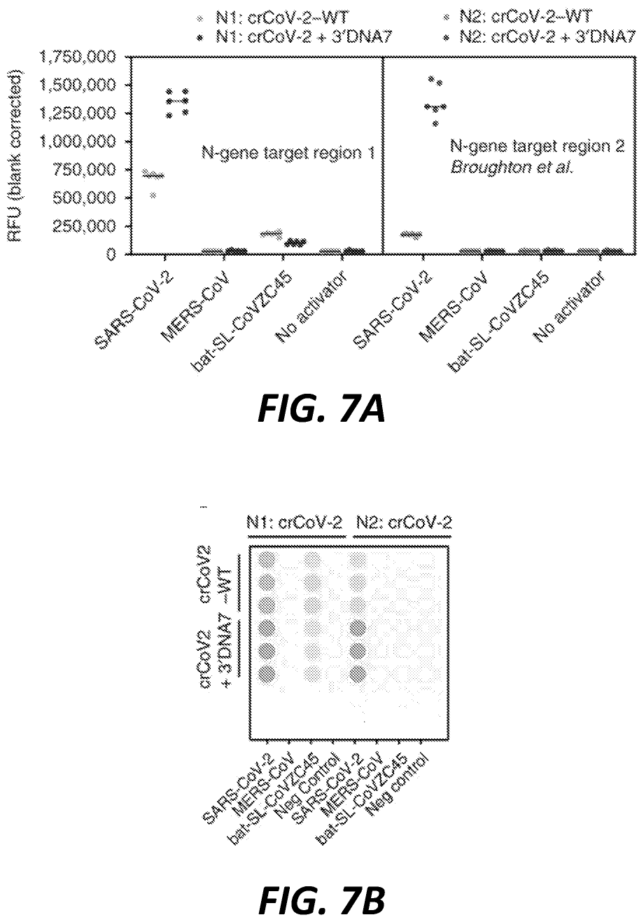

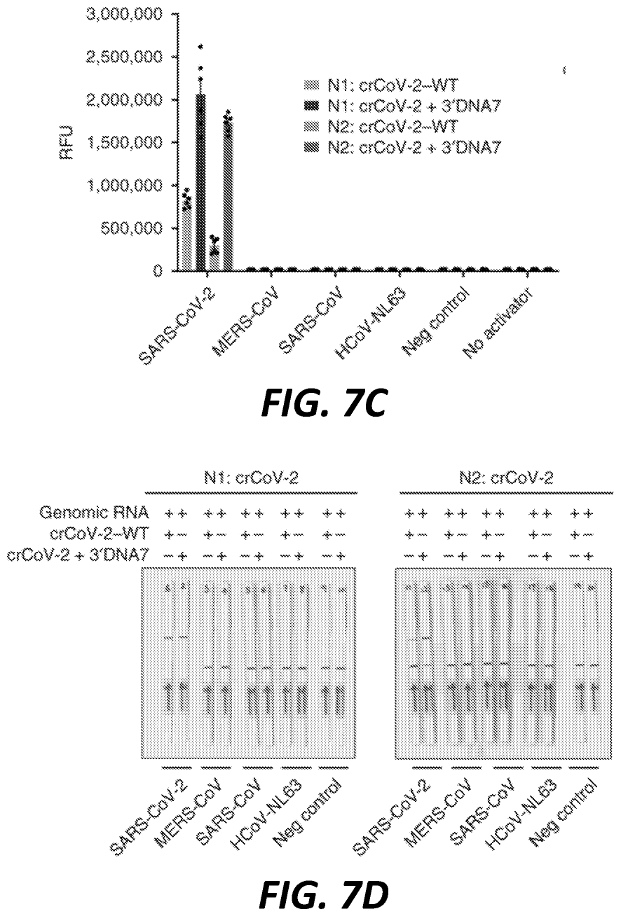

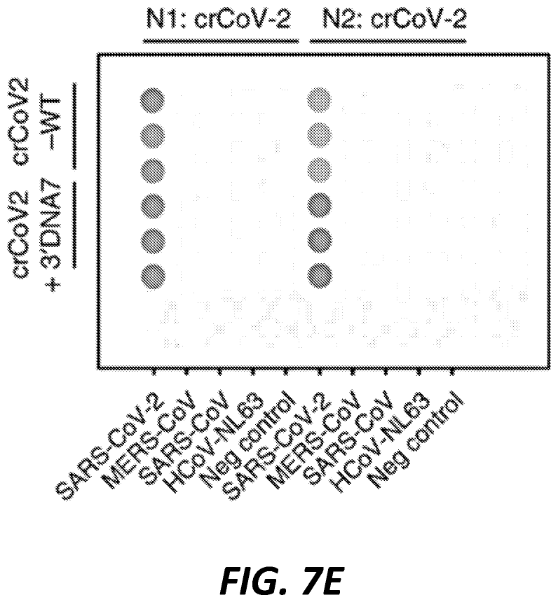

The specificity of the ENHANCE was investigated by testing crRNAs programmed to target SARS-CoV-2 against coronaviruses such as MERS-CoV, SARS-CoV, bat-SL-CoVZC45, and HCoV-NL63. Two guide RNAs were employed to target two different regions of the SARS-CoV-2 N-gene (referred to as N1 and N2 regions). The N1 region of SARS-CoV2 was selected to have ≤2 sequence mismatches with SARS-CoV and bat-SL-CoVZC45. This target region was therefore used to recognize if SARS-like coronaviruses strains are detected. The region N2 was selected from Broughton et al. that was specific for SARS-CoV-2 for exclusivity testing. SARS-CoV-2, MERS-CoV, and bat-SL-CoVZC45 plasmid controls (purchased from IDT) were targeted using these two crRNAs. The engineered N1:crCoV2+3′DNA7 and N2:crCoV2+3′DNA7 showed 3-fold and 7.8-fold higher in fluorescence signal compared to the wild-type N1:crCoV2-WT and N2:crCoV2-WT after 10 minutes of incubation, respectively. Notably, the engineered N1:crCoV2+3′DNA7 exhibited lower in fluorescence signal against MERS-CoV and bat-SL-CoVZC45, demonstrating 74% enhanced specificity towards SARS-CoV-2 (FIGS. 7A-B). Next, the two guides with clinically relevant extracted genomic RNAs of SARS-CoV-2, SARS-CoV Urbani, and HCoV63 (obtained from BEI resources) were tested. Both N1:crCoV+3′DNA7 and N2:crCoV+3′DNA7 showed specificity towards SARS-CoV-2 when an RT-LAMP step was applied (FIGS. 7C-E). This specificity was due to the fact that RT-LAMP primers sets were specific for SARS-CoV-2. Collectively, the ENHANCE system successfully retained the sequence matching fidelity when in complex with LbCas12a with enhanced specificity and significantly higher sensitivity compared to the wild-type crCoV2.

Very low copies of SARS-CoV-2 in both fluorescence-based and paper-based lateral flow assay platforms could be detected. When detecting the samples with low copies, it was observed that unmodified CRISPR exhibited a very small sensitivity ratio between the activator positive and the activator negative samples which led to difficulty in distinguishing if the target dsDNA was present in these samples. However, with the-ENHANCE, the activator positive samples displayed a very intense signal compared to activator negative samples, confirming a higher signal to noise ratio. The 7-mer DNA extension to crRNA is universal and spacer-independent, which means that it can be added to any crRNA without affecting the fidelity of the CRISPR/Cas12a system or significantly affecting the cost of synthesis.

### Example 12. The crRNA with a Toehold Hairpin Loop Possesses Enhanced Sensitivity and Specificity

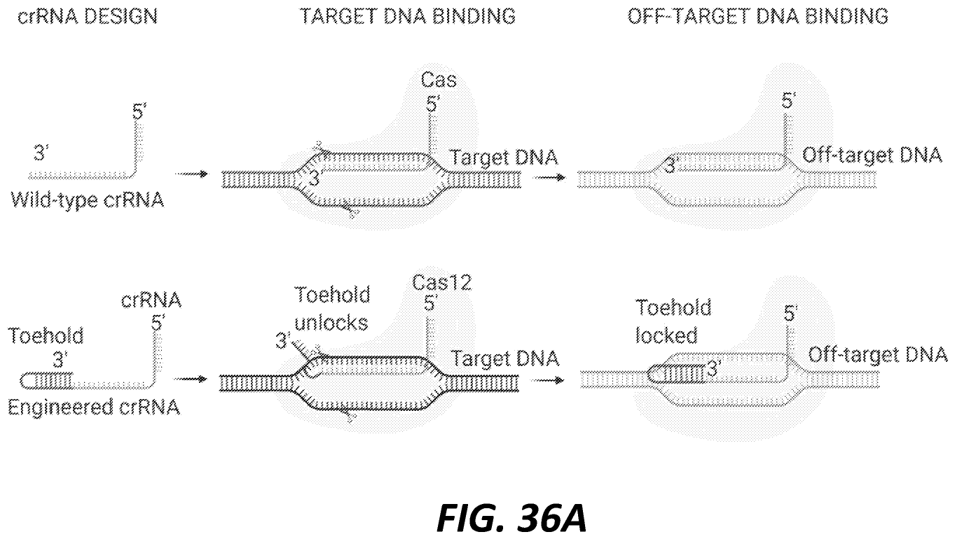

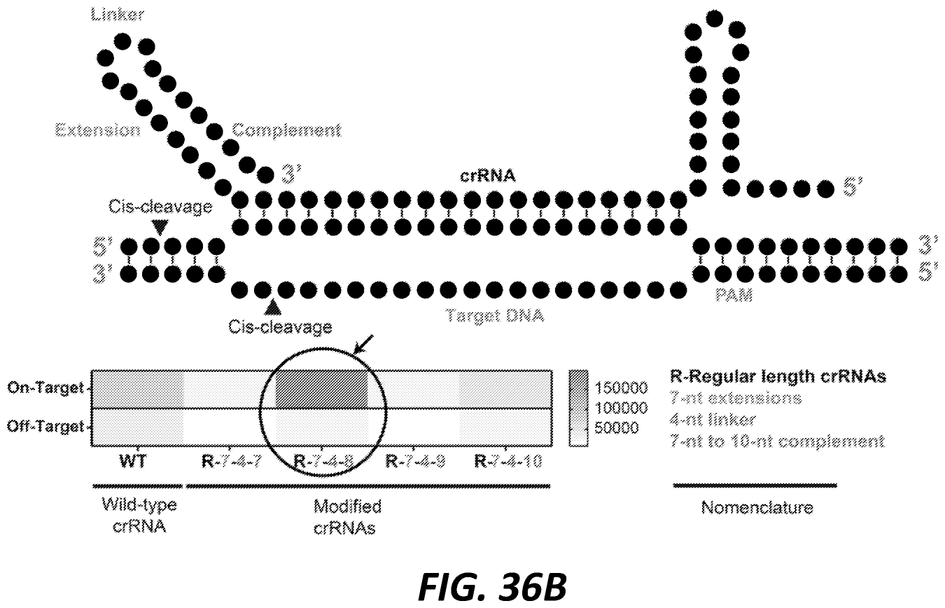

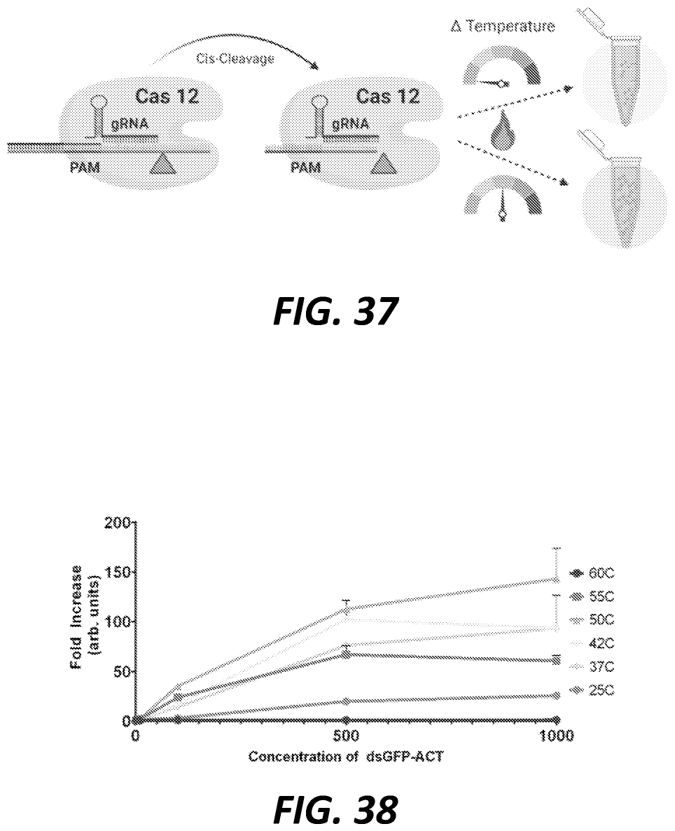

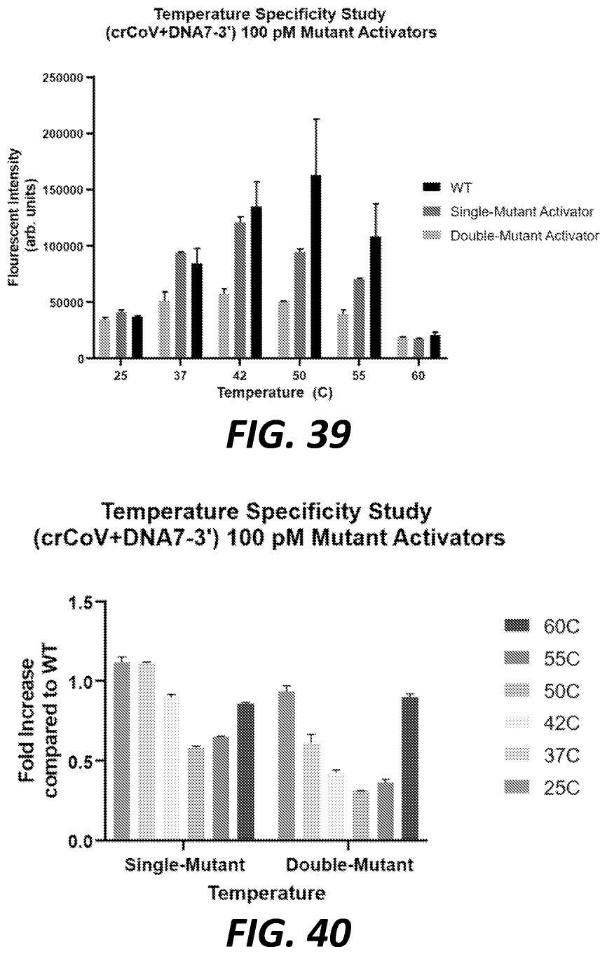

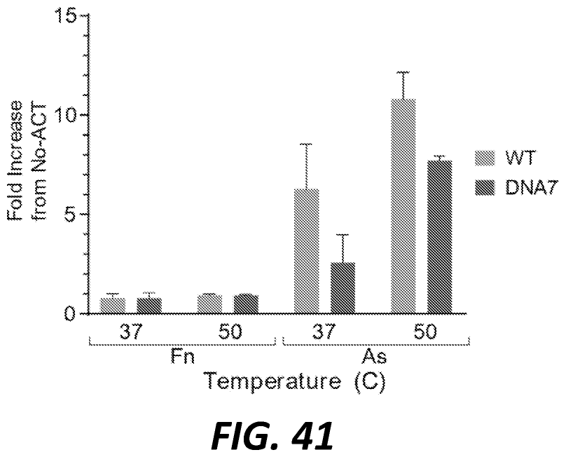

The 3′ end of the crRNA was extended such that they form a toehold hairpin loop (see FIGS. 36A-B). These secondary structures inhibit the off-target DNA interaction but can be unfolded by on-target DNA and can resolve single point mutation in the DNA. It was observed certain RNA extensions on the 3′ end of the crRNA maintains or improves sensitivity toward wildtype DNA but significantly reduces off-target cleavage. This data show that 3′ extensions on the crRNAs can improve specificity while maintaining or improving sensitivity. Furthermore, by adjusting the length of toehold sequences, the ratio of trans- to cis-cleavage can be significantly altered allowing for an increase in sensitivity or specificity. For instance, by increasing the ratio of trans- to cis-cleavage the toehold loops can allow more sensitive detection, while decreasing the trans- to cis-cleavage ratio would allow for more specific detection. Therefore 3′ end modifications will have huge implications for both disease detection as well as in vivo gene editing utilizing the CRISPR/Cas12a system.

The present disclosure further includes the following embodiments.

1A. A nucleic acid detection system comprising:
a CRISPR-associated (Cas) enzyme with trans cleavage activity;
a guide CRISPR RNA (crRNA) comprising a guide sequence and a polynucleotide extension sequence, wherein the guide sequence is configured to bind to a target polynucleotide, the polynucleotide extension sequence is linked to 3′-end of the guide sequence, and the polynucleotide extension sequence comprises a ssDNA or ssRNA with 1-19 nucleotides; and
a probe comprising an oligonucleotide element labeled with a detectable label, wherein a detectable signal or a detectable molecule is generated when the probe is cleaved by the CRISPR-associated enzyme.
   2A. The detection system of paragraph 1A, wherein the guide sequence is substantially complementary to the target polynucleotide.
   3A. The detection system of any one of the proceeding paragraphs, wherein a first end of the oligonucleotide element in the probe is linked to a fluorophore; a second end of the oligonucleotide element in the probe is linked to a quencher, optionally the probe further comprises biotin, optionally the crRNA comprises a fluorophore-quencher probe within the crRNA and the quencher is only cleaved in the presence of the target polynucleotide.
   4A. The detection system of any of the proceeding paragraphs, wherein a first end of the oligonucleotide element in the probe is linked to one fluorophore selected from the group consisting of FITC, HEX and FAM; a second end of the oligonucleotide element in the probe is linked to one quencher selected from the group consisting of BHQ1, BHQ2, MGBNFQ, and 3IABkFQ.
   5A. The detection system of any one of the proceeding paragraphs, wherein the probe comprises FAM-TTATT-3IABkFQ.
   6A. The detection system of paragraphs 1A or 2A, wherein the detectable label is a label selected from the group consisting of FAM-biotin, FITC-biotin, FAM-biotin-quencher, and FITC-biotin-quencher.
   7A. The detection system of paragraphs 3A or 4A, wherein the probe further comprises biotin.
   8A. The detection system of paragraphs 6A or 7A, wherein the probe comprises FAM-TTATTA(internal biotin)T-3IABkFQ.
   9A. The detection system of any one of the proceeding paragraphs, wherein the detectable label is FAM-biotin.
   10A. The detection system of any one of the proceeding paragraphs, wherein the detectable label is FITC-biotin.
   11A. The detection system of any one of the proceeding paragraphs, wherein the polynucleotide extension sequence is a ssDNA.
   12A. The detection system of paragraph 11A, wherein the ssDNA comprises at least 55% of A and/or T.
   13A. The detection system of paragraph 11A, wherein the ssDNA comprises at least 60% of A and/or T.
   14A. The detection system of paragraph 11A, wherein the ssDNA comprises at least 70% of A and/or T.
   15A. The detection system of paragraph 11A, wherein the ssDNA comprises at least 80% of A and/or T.
   16A. The detection system of paragraph 11A, wherein the ssDNA comprises at least 90% of A and/or T.
   17A. The detection system of paragraph 11A, wherein the ssDNA comprises at least 95% of A and/or T.
   18A. The detection system of paragraph 11A, wherein the ssDNA consists of A and/or T.
   19A. The detection system of paragraph 18A, wherein the ssDNA comprises a nucleotide sequence of TATTATT.
   20A. The detection system of any one of paragraphs 1A-10A, wherein the polynucleotide extension sequence comprises a ssRNA.
   21A. The detection system of any one of the proceeding paragraphs, wherein the polynucleotide extension sequence comprises 3-15 nucleotides.
   22A. The detection system of any one of the proceeding paragraphs, wherein the polynucleotide extension sequence comprises 4-13 nucleotides.
   23A. The detection system of any one of the proceeding paragraphs, wherein the polynucleotide extension sequence comprises 5-9 nucleotides.
   24A. The detection system of any one of the proceeding paragraphs, wherein the polynucleotide extension sequence comprises 7 nucleotides.
   25A. The detection system of any one of paragraphs 1A-4A, 6A, 7A, and 9A-24A, wherein the oligonucleotide element in the probe is a ssDNA or RNA.
   26A. The detection system of any one of the proceeding paragraphs, wherein the oligonucleotide element in the probe is a ssDNA.
   27A. The detection system of paragraph 26A, wherein the ssDNA in the probe comprises at least 55% of A and/or T.
   28A. The detection system of paragraph 26A, wherein the ssDNA in the probe comprises at least 60% of A and/or T.
   29A. The detection system of paragraph 26A, wherein the ssDNA in the probe comprises at least 70% of A and/or T.
   30A. The detection system of paragraph 26A, wherein the ssDNA in the probe comprises at least 80% of A and/or T.
   31A. The detection system of paragraph 26A, wherein the ssDNA in the probe comprises at least 90% of A and/or T.
   32A. The detection system of paragraph 26A, wherein the ssDNA in the probe comprises at least 95% of A and/or T.
   33A. The detection system of paragraph 26A, wherein the ssDNA in the probe consists of A and/or T.
   34A. The detection system of paragraph 26A, wherein the ssDNA in the probe comprises a nucleotide sequence of TTATT.
   35A. The detection system of any one of the proceeding paragraphs, wherein the CRISPR-associated enzyme is Cas12a or any enzyme having trans-cleavage activity comparable to Cas12a.
   36A. The detection system of any one of the proceeding paragraphs, wherein the CRISPR-associated enzyme is Cas12a or Cas12b.
   37A. The detection system of any one of the proceeding paragraphs, wherein the CRISPR-associated enzyme is Cas12a.
   38A. The detection system of any one of the proceeding paragraphs, wherein the CRISPR-associated enzyme is selected from the group consisting of PcCas12a, MbCas12a, FnCas12a and LbCas12a, optionally the CRISPR-associated enzyme is FnCas12a or LbCas12a.
   39A. The detection system of any one of the proceeding paragraphs, wherein the CRISPR-associated enzyme is LbCas12a.
   40A. The detection system of any one of the proceeding paragraphs, wherein the target polynucleotide is a DNA.
   41A. The detection system of paragraph 40A, wherein the DNA is a ssDNA, a dsDNA, or a heteroduplex of RNA and DNA.
   42A. The detection system of paragraph 40A, wherein the DNA is a ssDNA.
   43A. The detection system of any one of the proceeding paragraphs, further comprising a nucleic acid amplification reagent.
   44A. The detection system of paragraph 43A, wherein the reagent is for an amplification selected from the group consisting of nucleic acid sequence-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), PCR, multiple displacement amplification (MDA), rolling circle amplification (RCA), ligase chain reaction (LCR), and ramification amplification method (RAM).
   45A. The detection system of paragraph 44A, wherein the reagent is for an amplification selected from the group consisting of recombinase polymerase amplification (RPA), and loop-mediated isothermal amplification (LAMP).
   46A. The detection system of any one of the proceeding paragraphs, wherein the system comprises two guide CRISPR RNAs (crRNA) and two polynucleotide targets, wherein a first crRNA comprising a first target polynucleotide configured to bind to a first target polynucleotide, the first target polynucleotide is a DNA fragment synthesized from a RNA fragment in N1 region of SARS-CoV-2; and a second crRNA comprising a second target polynucleotide configured to bind to a second target polynucleotide, the second target polynucleotide is a DNA fragment synthesized from a RNA fragment in N2 region of SARS-CoV-2.
   47A. The detection system of any one of paragraphs 1A-45A, wherein the target polynucleotide is at least one selected from the group consisting of Control genes: RNase P (P1 & P2), GFP, HIV Tat gene, HCV gene encoding a polyprotein precursor PCA3 (Prostate cancer Antigen 3), and SARS-CoV-2 genes: N (N1 & N2), E gene (E1 & E2), and RdRp gene (R1 & R2).
   48A. The detection system of any one of the proceeding paragraphs, wherein the guide sequence is complementary to a target polynucleotide that is diagnostic for a disease state.
   49A. The detection system of paragraph 48A, wherein the disease state is cancer.
   50A. The detection system of paragraph 48A, wherein the disease state is a genetic disease or disorder.
   51A. The detection system of paragraph 48A, wherein the disease state is an infection.
   52A. The detection system of paragraph 48A, wherein the infection is caused by a virus, a bacterium, a fungus, protozoan, or a parasite.
   53A. The detection system of paragraph 48A, wherein the infection is a viral infection.
   54A. The detection system of paragraph 52A, wherein the infection is caused by SARS-CoV-2.
   55A. The detection system of any one of the proceeding paragraphs, further comprising a divalent metal cation.
   56A. The detection system of paragraph 55A, wherein the divalent metal cation is magnesium.
   57A. The detection system of paragraph 55A, wherein the divalent metal cation is calcium.
   58A. The detection system of any one of the proceeding paragraphs, wherein the target polynucleotide comprises a modified nucleotide.
   59A. The detection system of paragraph 58A, wherein the modified nucleotide is a methylated nucleotide.
   60A. The detection system of any one of the proceeding paragraphs, further comprising a buffer.
   61A. The nucleic acid detection system of any one of paragraphs 1A-60A, wherein the crRNA further comprises a linker having two ends, one end of the linker linked to 3′-end of the polynucleotide extension sequence, and the other end of the linker linked to a complementary sequence, wherein the complementary sequence is complementary to the extension sequence, wherein the extension sequence, the linker, and the complementary sequence form a toehold conformation, and the toehold conformation unfolds when the guide sequence binds to the target polynucleotide.
   62A. The nucleic acid detection system of any one of paragraph 1A-60A, wherein the polynucleotide extension sequence comprises a linker having two ends, one end of the linker linked to 3′-end of the guide sequence, and the other end of the linker linked to a complementary sequence, wherein the complementary sequence is complementary to at least a 3′ portion of the guide sequence, wherein the linker, the complementary sequence, and the 3′ portion of the guide sequence form a toehold conformation, and the toehold conformation unfolds when the guide sequence binds to the target polynucleotide.
   63A. A lateral flow device, comprising
a sample loading portion; and
the detection system of any one of paragraphs 1A-62A.
   64A. The lateral flow device of paragraph 63A, wherein the sample loading portion comprises an amplification reagent to amplify the target polynucleotide.
   65A. A kit for detecting a virus, comprising:
the detection system of any one of paragraphs 1A-62A; and
instructions for use of the detection system for detecting the virus.
   66A. The kit of paragraph 65A, further comprising a test strip for detecting the detectable molecule.
   67A. The kit of paragraph 66A, wherein test strip comprises a colorimetric paper-based assay or an electrochemical biosensor.
   68A. A kit for detecting a target polynucleotide, comprising
the detection system of any one of paragraphs 1A-62A; and
instructions for use of the detection system for detecting the target polynucleotide.
   69A. A ribonucleoprotein complex, comprising:
a CRISPR-associated enzyme with trans cleavage activity; and
a guide CRISPR RNA (crRNA) comprising a guide sequence and a polynucleotide extension sequence, wherein the guide sequence is configured to bind to a target polynucleotide, the polynucleotide extension sequence is linked to 3′-end of the guide sequence, and the polynucleotide extension sequence comprises a ssDNA or ssRNA with 1-19 nucleotides.
   70A. A method of detecting a target polynucleotide in a sample obtained from a subject, the method comprising:
contacting the sample with the detection system of paragraphs 1A-60A, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and
detecting the signal or the molecule, wherein detection of the signal or the molecule indicates presence of the target polynucleotide in the sample.
   71A. The method of paragraph 70A, wherein the detection system is on a substrate, and wherein the substrate is exposed to the sample.
   72A. The method of paragraph 71A, wherein the substrate comprises a flexible material.
   73A. The method of paragraphs 71A or 72A, wherein the substrate is selected from the group consisting of: a paper substrate, a fabric substrate, and a polymer-based substrate.
   74A. The method of any one of paragraphs 70A-73A, wherein the sample comprises saliva, respiratory secretions, exudate, blood, plasma, urine, stool, and sera.
   75A. The method of any one of paragraphs 70A-74A, further comprising amplifying the target polynucleotide in the sample prior to contacting the sample with the detection system.
   76A. The method of paragraph 75A, wherein amplifying the target polynucleotide comprises isothermal amplification.
   77A. The method of paragraph 70A, wherein the detectable molecule is detected by a test strip.
   78A. A method of detecting a condition in a subject, comprising;
contacting a sample from the subject with the detection system of paragraphs 1-52, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and
detecting the signal or the molecule, wherein detection of the signal or the molecule indicates presence of the condition.
   79A. The method of paragraph 78A, wherein the sample is urine, sera, blood, respiratory secretions, exudate, stool or saliva.
   80A. The method of paragraphs 78A or 79A, wherein the sample and the detection system are incubated at a temperature of 20-60° C. for a period of time, optionally the detection is performed in one-pot or multiple-pot forms.
   81A. The method of paragraph 78A or 79A, wherein the sample and the detection system are incubated at a temperature of 30-60° C. for a period of time.
   82A. The method of paragraph 78A or 79A, wherein the sample and the detection system are incubated at a temperature of 40-60° C. for a period of time.
   83A. The method of paragraph 78A or 79A, wherein the sample and the detection system are incubated at a temperature of 40-55° C. for a period of time, optionally the sample and the detection system are incubated at a temperature of about 42° C., 50° C., or 55° C.
   84A. The method of paragraphs 78A-83A, wherein detecting the signal or the molecule occurs within 10 minutes of contacting the sample with the detection system.
   85A. A method of detecting a target polynucleotide in a sample obtained from a subject, comprising:
contacting a sample from the subject with a detection system, wherein the detection system comprises a CRISPR-associated (Cas) enzyme with trans cleavage activity, a guide CRISPR RNA (crRNA) comprising a guide sequence, and a probe comprising an oligonucleotide element labeled with a detectable label, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and
detecting the signal or the molecule, wherein detection of the signal or the molecule indicates presence of the target polynucleotide in the sample;
wherein the sample and the detection system are incubated at a temperature of 30-70° C. for a period of time.
   86A. The method of paragraph 85A, wherein the sample and the detection system are incubated at a temperature of 30-60° C. for a period of time.
   87A. The method of paragraph 85A, wherein the sample and the detection system are incubated at a temperature of 35-60° C. for a period of time.
   88A. The method of paragraph 85A, wherein the sample and the detection system are incubated at a temperature of 40-55° C. for a period of time.
   89A. The method of paragraph 83A, wherein the sample and the detection system are incubated at a temperature of 40-45° C. for a period of time.
   90A. The method of paragraph 85A, wherein the sample and the detection system are incubated at a temperature of 45-50° C. for a period of time.
   91A. The method of paragraph 85A, wherein the sample and the detection system are incubated at a temperature of 50-55° C. for a period of time.
   92A. A modified CRISPR-associated (Cas) complex comprising:
a Cas12 enzyme;
a guide CRISPR RNA (crRNA) comprising a guide sequence and a polynucleotide extension sequence, wherein the guide sequence is configured to bind to a target polynucleotide, the polynucleotide extension sequence is linked to 3′-end of the guide sequence, and the polynucleotide extension sequence comprises a ssDNA or ssRNA with 1-19 nucleotides.

Various modifications and variations of the described methods, systems, and kits of the present invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

The following tables provide the sequences of nucleic acids used in the examples.

| | | |
|---|---|---|
| Sequence | | SEQ ID |
| Name | Sequence | NO: |

CRISPR RNAs (crRNAs)
Selected crRNAs for AsCas12a

| | | |
|---|---|---|
| AscrCon | /AlTR1/rUrArA rUrUrU rCrUrA rCrUrC rUrUrG rUrArG rArUrC | 1 |
| | rGrUrU rArArU rCrGrC rGrUrA rUrArA rUrArC rGrG/AlTR2/ | |
| AscrGFP1 | /AltR1/rUrArA rUrUrU rCrUrA rCrUrC rUrUrG rUrArG rArUrC rGrUrC | 2 |
| | rGrCrC rGrUrC rCrArG rCrUrC rGrArC rC/AltR2/ | |
| AscrGFP2- | /AltR1/rUrArA rUrUrU rCrUrA rCrUrC rUrUrG rUrArG rArUrC rUrCrA | 3 |
| WT | rGrGrG rCrGrG rArCrU rGrGrG rUrGrC rU/AltR2/ | |
| AscrGFP2- | rUrArA rUrUrU rCrUrA rCrUrC rUrUrG rUrArG rArUrC rUrCrA | 4 |
| WT-no-Alt | rGrGrG rCrGrG rArCrU rGrGrG rUrGrC rU | |
| AscrGFP2+ | rUrArA rUrUrU rCrUrA rCrUrC rUrUrG rUrArG rArUrC rUrCrA rGrGrG | 5 |
| 3′DNA7 | rCrGrG rArCrU rGrGrG rUrGrC rUTA TTA TT | |

Selected crRNAs for FnCas12a

| | | |
|---|---|---|
| FncrGFP2- | rUrArA rUrUrU rCrUrA rCrUrG rUrUrG rUrArG rArUrC rUrCrA | SEQ ID |
| WT | rGrGrG rCrGrG rArCrU rGrGrG rUrGrC rU | NO: 6 |
| FncrGFP2+ | rUrArA rUrUrU rCrUrA rCrUrG rUrUrG rUrArG rArUrC rUrCrA rGrGrG | SEQ ID |
| 3′DNA7 | rCrGrG rArCrU rGrGrG rUrGrC rUTA TTA TT | NO: 7 |
| FncrPCA3 | rUrArA rUrUrU rCrUrA rCrUrG rUrUrG rUrArG rArUrU rCrArC rCrCrC | SEQ ID |
| | rUrGrC rCrArU rUrGrA rGrArU rG | NO: 8 |

Selected crNRAs for LbCas12a

| | | |
|---|---|---|
| LbcrCon | rUrArA rUrUrU rCrUrA rCrUrA rArGrU rGrUrA rGrArU rCrGrU rUrArA | SEQ ID |
| (Neg-Ctrl- | rUrCrG rCrGrU rArUrA rArUrA rCrGrG | NO: 9 |
| LbcrRNA) | | |
| LbcrGFP1 | rUrArA rUrUrU rCrUrA rCrUrA rArGrU rGrUrA rGrArU rCrGrU rCrGrC | SEQ ID |
| | rCrGrU rCrCrA rGrCrU rCrGrA rCrC | NO: 10 |
| LbcrGFP2- | rUrArA rUrUrU rCrUrA rCrUrA rArGrU rGrUrA rGrArU rCrUrC rArGrG | SEQ ID |
| WT | rGrCrG rGrArC rUrGrG rGrUrG rCrU | NO: 11 |
| LbcrPCA3- | rUrArA rUrUrU rCrUrA rCrUrA rArGrU rGrUrA rGrArU rUrCrA rCrCrC | SEQ ID |
| WT | rCrUrG rCrCrA rUrUrG rArGrA rUrG | NO: 12 |
| LbcrPCA3+ | rUrArA rUrUrU rCrUrA rCrUrA rArGrU rGrUrA rGrArU rUrCrA rCrCrC | SEQ ID |
| 3′DNA7 | rCrUrG rCrCrA rUrUrG rArGrA rUrGT ATT ATT | NO: 13 |
| LbcrHIV- | TUrArArUrUrUrCrUrArCrUrArArGrUrGrUrArGrArUrCrCrUrUrGrGrUrG | SEQ ID |
| WT | rGrGrUrGrCrUrArCrUrCrCrU | NO: 14 |
| LbcrHIV+3′ | rUrArArUrUrUrCrUrArCrUrArArGrUrGrUrArGrArUrCrCrUrUrGrGrUrG | |
| DNA7 | rGrGrUrGrCrUrArCrUrCrCrUTATTATT | NO: 15 |
| LbcrHCV- | rUrArArUrUrUrCrUrArCrUrArArGrUrGrUrArGrArUrUrGrCrUrCrArUrG | SEQ ID |
| WT | rArUrGrCrArCrGrGrUrCrUrA | NO: 16 |
| LbcrHCV+ | rUrArArUrUrUrCrUrArCrUrArArGrUrGrUrArGrArUrUrGrCrUrCrArUr | SEQ ID |
| 3′DNA7 | GrArUrGrCrArCrGrGrUrCrUrATATTATT | NO: 17 |
| N1:crCoV- | rUrArArUrUrUrCrUrArCrUrArArGrUrGrUrArGrArUrGrUrGrGrArCrCr | SEQ ID |
| 2-WT | CrUrCrArGrArUrUrCrArArCrU | NO: 18 |
| N1:crCoV- | rUrArArUrUrUrCrUrArCrUrArArGrUrGrUrArGrArUrGrUrGrGrArCrCr | SEQ ID |
| 2+3′DNA7 | CrUrCrArGrArUrUrCrArArCrUTATTATT | NO: 19 |
| N2:crCoV- | rUrArArUrUrrCrUrArCrUrArArGrUrGrUrArGrArUrCrCrCrCrCrAtGrC | SEQ ID |
| 2-WT | GrCrUrUrCrArGrCrGrUrUrC | NO: 20 |
| N2:crCoV- | rUrArArUrUrUrCrUrArCrUrArArGrUrGrUrArGrArUrCrCrCrCrCrArGrC | SEQ ID |
| 2+3′DNA7 | rGrCrUrUrCrArGrCrGrUrUrCTATTATT | NO: 21 |

3′ DNA modified crGFP2 for LbCas12a

| | | |
|---|---|---|
| LbcrGFP2+3′DNA7 | rUrArA rUrUrU rCrUrA rCrUrA rArGrU rGrUrA rGrArU | 22 |
| | rCrUrC rArGrG rGrCrG rGrArC rUrGrG rGrUrG rCrUT | |
| | ATT ATT | |
| LbcrGFP2+3′DNA13 | rUrArA rUrUrU rCrUrA rCrUrA rArGrU rGrUrA rGrArU | 23 |
| | rCrUrC rArGrG rGrCrG rGrArC rUrGrG rGrUrG rCrUT | |
| | ATT ATT ATT ATT | |
| LbcrGFP2+3′DNA19 | rUrArA rUrUrU rCrUrA rCrUrA rArGrU rGrUrA rGrArU | 24 |
| | rCrUrC rArGrG rGrCrG rGrArC rUrGrG rGrUrG rCrUT | |
| | ATT ATT ATT ATT ATT ATT | |
| LbcrGFP2+3′DNA31 | rUrArA rUrUrU rCrUrA rCrUrA rArGrU rGrUrA rGrArU | 25 |
| | rCrUrC rArGrG rGrCrG rGrArC rUrGrG rGrUrG rCrUT | |
| | ATT ATT ATT ATT ATT ATT ATT ATT ATT ATT | |
| LbcrGFP2+3′DNA7(GC) | rUrArA rUrUrU rCrUrA rCrUrA rArGrU rGrUrA rGrArU | 26 |
| | rCrUrC rArGrG rGrCrG rGrArC rUrGrG rGrUrG rCrUC | |
| | GCC GCC | |

5′ DNA modified crGFP2 or LbCas12a

| | | |
|---|---|---|
| LbcrGFP2+5′DNA7 | TTA TTA TrUrA rArUrU rUrCrU rArCrU rArArG rUrGrU | 27 |
| | rArGrA rUrCrU rCrArG rGrGrC rGrGrA rCrUrG rGrGrU | |
| | rGrCrU | |
| Tru- | TTA TTA TrArA rUrUrU rCrUrA rCrUrA rArGrU rGrUrA | 28 |
| LbcrGFP2+5′DNA7 | rGrArU rCrUrC rArGrG rGrCrG rGrArC rUrGrG rGrUrG | |
| | rCrU | |
| LbcrGFP2+5′DNA13 | TTA TTA TTA TTA TrUrA rArUrU rUrCrU rArCrU rArArG | 29 |
| | rUrGrU rArGrA rUrCrU rCrArG rGrGrC rGrGrA rCrUrG | |
| | rGrGrU rGrCrU | |
| LbcrGFP2+5′DNA19 | TTA TTA TTA TTA TTA TTA TrUrA rArUrU rUrCrU | 30 |
| | rArCrU rArArG rUrGrU rArGrA rUrCrU rCrArG rGrGrC | |
| | rGrGrA rCrUrG rGrGrU rGrCrU | |
| LbcrGFP2+5′DNA31 | TTA TTA TTA TTA TTA TTA TTA TTA TTA TTA TrUrA | 31 |
| | rArUrU rUrCrU rArCrU rArArG rUrGrU rArGrA rUrCrU | |
| | rCrArG rGrGrC rGrGrA rCrUrG rGrGrU rGrCrU | |

3′ PSDNA modified crGFP2 for LbCas12a

| | | |
|---|---|---|
| LbcrGFP2+3′PSDNA7 | rUrArA rUrUrU rCrUrA rCrUrA rArGrU rGrUrA rGrArU | 32 |
| | rCrUrC rArGrG rGrCrG rGrArC rUrGrG rGrUrG rCrU*T* | |
| | A*T*T* A*T*T | |
| LbcrGFP2+3′PSDNA13 | rUrArA rUrUrU rCrUrA rCrUrA rArGrU rGrUrA rGrArU | 33 |
| | rCrUrC rArGrG rGrCrG rGrArC rUrGrG rGrUrG rCrU*T* | |
| | A*T*T* A*T*T* A*T*T* A*T*T | |
| LbcrGFP2+3′PSDNA19 | rUrArA rUrUrU rCrUrA rCrUrA rArGrU rGrUrA rGrArU | 34 |
| | rCrUrC rArGrG rGrCrG rGrArC rUrGrG rGrUrG rCrU*T* | |
| | A*T*T* A*T*T* A*T*T* A*T*T* A*T*T* A*T*T | |
| LbcrGFP+6DNA+1PS-3′ | rUrArA rUrUrU rCrUrA rCrUrA rArGrU rGrUrA rGrArU | 35 |
| | rCrUrC rArGrG rGrCrG rGrArC rUrGrG rGrUrG rCrUT | |
| | ATT AT*T | |
| LbCrGFP+5DNA+2PS-3′ | rUrArA rUrUrU rCrUrA rCrUrA rArGrU rGrUrA rGrArU | 36 |
| | rCrUrC rArGrG rGrCrG rGrArC rUrGrG rGrUrG rCrUT | |
| | ATT A*T*T | |
| LbCrGFP+4DNA+3PS-3′ | rUrArA rUrUrU rCrUrA rCrUrA rArGrU rGrUrA rGrArU | 37 |
| | rCrUrC rArGrG rGrCrG rGrArC rUrGrG rGrUrG rCrUT | |
| | ATT* A*T*T | |
| LbCrGFP+3DNA+4PS-3′ | rUrArA rUrUrU rCrUrA rCrUrA rArGrU rGrUrA rGrArU | 38 |
| | rCrUrC rArGrG rGrCrG rGrArC rUrGrG rGrUrG rCrUT | |
| | AT*T* A*T*T | |
| LbCrGFP+2DNA+5PS-3′ | rUrArA rUrUrU rCrUrA rCrUrA rArGrU rGrUrA rGrArU | 39 |
| | rCrUrC rArGrG rGrCrG rGrArC rUrGrG rGrUrG rCrUT | |
| | A*T*T* A*T*T | |
| LbCrGFP+1DNA+6PS-3′ | rUrArA rUrUrU rCrUrA rCrUrA rArGrU rGrUrA rGrArU | 40 |
| | rCrUrC rArGrG rGrCrG rGrArC rUrGrG rGrUrG rCrUT* | |
| | A*T*T* A*T*T | |

5′ PSDNA modified crGFP2 for LbCas12a

| | | |
|---|---|---|
| LbcrGFP2+5′PSDNA7 | T*T*A* T*T*A* T*rUrA rArUrU rUrCrU rArCrU rArArG | 41 |
| | rUrGrU rArGrA rUrCrU rCrArG rGrGrC rGrGrA rCrUrG | |
| | rGrGrU rGrCrU | |
| LbcrGFP2+5′PSDNA13 | T*T*A* T*T*A* T*T*A* T*T*A* T*rUrA rArUrU rUrCrU | 42 |
| | rArCrU rArArG rUrGrU rArGrA rUrCrU rCrArG rGrGrC | |
| | rGrGrA rCrUrG rGrGrU rGrCrU | |
| EbcrGFP2+5′PSDNA19 | T*T*A* T*T*A* T*T*A* T*T*A* T*T*A* T*T*A* | 43 |
| | T*rUrA rArUrU rUrCrU rArCrU rArArG rUrGrU rArGrA | |
| | rUrCrU rCrArG rGrGrC rGrGrA rCrUrG rGrGrU rGrCrU | |

3 ′ RNA modified crGFP2 or LbCas12a

| | | |
|---|---|---|
| LbcrGFP2+3′RNA7 | rUrArA rUrUrU rCrUrA rCrUrA rArGrU rGrUrA rGrArU | 44 |
| | rCrUrC rArGrG rGrCrG rGrArC rUrGrG rGrUrG rCrUrU | |
| | rArUrU rArUrU | |
| LbcrGFP2+3′RNA13 | rUrArA rUrUrU rCrUrA rCrUrA rArGrU rGrUrA rGrArU | 45 |
| | rCrUrC rArGrG rGrCrG rGrArC rUrGrG rGrUrG rCrUrU | |
| | rArUrU rArUrU rArUrU rArUrU | |
| LbcrGFP2+3′RNA19 | rUrArA rUrUrU rCrUrA rCrUrA rArGrU rGrUrA rGrArU | 46 |
| | rCrUrC rArGrG rGrCrG rGrArC rUrGrG rGrUrG rCrUrU | |
| | rArUrU rArUrU rArUrU rArUrU rArUrU rArUrU | |

5 ′ RNA modified crGFP2 or LbCas12a

| | | |
|---|---|---|
| LbcrGFP2+5′RNA7 | rUrUrA rUrUrA rUrUrA rArUrU rUrCrU rArCrU rArArG | 47 |
| | rUrGrU rArGrA rUrCrU rCrArG rGrGrC rGrGrA rCrUrG | |
| | rGrGrU rGrCrU | |
| LbcrGFP2+5′RNA13 | rUrUrA rUrUrA rUrUrA rUrUrA rUrUrA rArUrU rUrCrU | 48 |
| | rArCrU rArArG rUrGrU rArGrA rUrCrU rCrArG rGrGrC | |
| | rGrGrA rCrUrG rGrGrU rGrCrU | |
| LbcrGFP2+5′RNA19 | rUrUrA rUrUrA rUrUrA rUrUrA rUrUrA rUrUrA rUrUrA | 49 |
| | rArUrU rUrCrU rArCrU rArArG rUrGrU rArGrA rUrCrU | |
| | rCrArG rGrGrC rGrGrA rCrUrG rGrGrU rGrCrU | |

Activator DNA&RNA

| | | |
|---|---|---|
| DD3/PCA3-40nt-T- | AGA CTA CAG ACA TCT CAA TGG CAG GGG TGA | 50 |
| ssDNA | GAA ATA AGA A | |
| DD3/PCA3-40nt-T- | TTC TTA TTT CTC ACC CCT GCC ATT GAG ATG TCT | 51 |
| ssDNA-NT | GTA GTC T | |
| TTATT sequence-13 mer | TATTATTATTATT | 52 |
| DD3-PCA3-gene- | CAA GAT AAA TAA GTG AAG AGC TAG TCC GCT | 53 |
| 25 Obp-transcript variant | GTG AGT CTC CTC AGT GAC ACA GGG CTG GAT CAC | |
| 1 | CAT CGA CGG CAC TTT CTG AGT ACT CAG TGC AGC | |
| | AAA GAA AGA CTA CAG ACA TCT CAA TGG CAG | |
| | GGG TGA GAA ATA AGA AAG GCT GCT GAC TFT | |
| | ACC ATC TGA GGC CAC ACA TCT GCT GAA ATG GAG | |
| | ATA ATT AAC ATC ACT AGA AAC AGC AAG ATG | |
| | ACA ATA TAA TGT CTA AGT AGT GAC ATG TTT T | |
| LbCas12a-Activator- | rUrUrCrUrCrUrCrUrGrCrArCrCrArCrUrCrUrUrCrUrCrUrUr | 54 |
| HIV 1-RNA | UrGrCrCrUrUrGrGrUrGrGrGrUrGrCrUrArCrUrCrCrUrArAr | |
| | UrGrGrUrUrCrArArUrUrUrU | |
| LbCas12a-HIV-DNA-T- | AAAATTGAACCATTAGGAGTAGCACCCACCAAGGCA | 55 |
| Activator | AAGAGAAGAGTGGTGCAGAGAGAA | |
| LbCas12a-HIV-DNA- | TTCTCTCTGCACCACTCTTCTCTTTGCCTTGGTGGGTG | 56 |
| NT-Activator | CTACTCCTAATGGTTCAATTTT | |
| HCV_RNA_reference_ | rGrCrCrUrUrGrUrGrGrUrArCrUrGrCrCrUrGrArUrArGrGrG | 57 |
| Genome | ᵣUrGrCrUrUrGrCrGrArGrUrGrCrCrCrCrGrGrGrArGrGrUrC | |
| (Polyprotein precursor) | TUrCrGrUrArGrArCrCrGrUrGrCrArUrCrArUrGrArGrCrArC | |
| | rArArArUrCrCrUrArArArCrCrUrC | |
| HCV_DNA_act_T | CCTCTAATACGACTCACTATAGGCGTTGGGTTGCGAA | 58 |
| | CGGCCTTGTGGTACTGCCTGATAGGGTGCTTGCGAGT | |
| | GCCCCGGGAGGTCTCGTAGACCGTGCATCATGAGCA | |
| | CAAATCCTAAACCTC | |
| HCV_DNA_act_NT | GAGGTTTAGGATTTGTGCTCATGATGCACGGTCTACG | 59 |
| | AGACCTCCCGGGGCACTCGCAAGCACCCTATCAGGC | |
| | AGTACCACAAGGCCGTTCGCAACCCAACGCCTATAG | |
| | TGAGTCGTATTAGAGG | |
| 2019-nCoV_N_Positive | Plasmid CAT_10006625_2019-nCoV_N_Positive Control | |
| Control | from IDT | |
| MERS-CoV Control | Plasmid MERS-CoV Control from IDT | |
| Bat-SL-CoVZC45 | Plasmid SARS-CoV Control from IDT | |
| Control | | |

Activator Primers

| | | |
|---|---|---|
| GFP-Act-NT-MedC | GGG GTC TTT G/iMe-dC/T ZiMe-dC/AG GG/iMe-dC/ GGA | 60 |
| | /iMe-dC/TG GGT G/iMe-dC/T CAG GTA GTG G | |
| GFP-Act-T-MedC | CCA CTA CCT GAG ZiMe-dC/A/iMe-dC/ /iMe-dC//iMe- | 61 |
| | dC/A GT/iMe-dC/ /iMe-dC/G/Me-dC/ /iMe-dC//iMe-dC/T | |
| | GAG CAA AGA CCC C | |
| GFP-40nt-T- | CCA CTA CCT GrArG rCrArC rCrCrA rGrUrC rCrGrC | 62 |
| heteroDNA-RNA | rCrCrU rGrArG CAA AGA CCC C | |
| GFP-40nt-NT- | GGG GTC TTT GrCrU rCrArG rGrGrC rGrGrA rCrUrG | 63 |
| heteroDNA-RNA | rGrGrU rGrCrU CAG GTA GTG G | |
| HIV_RNA_Primer_RT- | AAA ATT GAA CCA TFA GGA GTA GC | 64 |
| Superscript | | |
| HIV_RNA_Primer_RT- | AAA ATT GAA CCA TFA GG | 65 |
| MMLV | | |
| HIV-RNA-RP | TTC TCT CTG CAC CAC TCT TC | 66 |
| HCV_RP_gblock_also_ | GAG GTT TAG GAT TTG TGC TCA T | 67 |
| RT1_LN | | |
| HCV_RP_gblock_also_ | GAG GTT TAG GAT TTG TGC TCA TGA | 68 |
| RT2_LN | | |
| HCV_FP_1 | CCT CTA ATA CGA CTC ACT A | 69 |
| HCV_FP_2 | CCT CTA ATA CGA CTC A | 70 |
| HCV_PCR_cDNA_FP1 | GCG TTG GGT TGC GAA CGG CC | 71 |
| HCV_PCR_cDNA_FP2 | GCG TTG GGT TGC GAA CGG | 72 |
| HIV_RT_cDNA_FP | GCC TTG TGG TAC TGC CTG AT | 73 |
| 2019-nCoV N1_FP | GAC CCC AAA ATC AGC GAA AT | 74 |
| 2019-nCoV N1_RP | TCT GGT TAC TGC CAG TTG AAT CTG | 75 |
| 2019-nCoV_N1_T7FP | CCT CTA ATA CGA CTC ACT ATA GGA CCC CAA AAT | 76 |
| | CAG CGA AAT | |
| 2019-nCoV_N1_T7RP | CCT CTA ATA CGA CTC ACT ATA GGT CTG GTT ACT | 77 |
| | GCC AGT TGA ATC TG | |
| 2019-nCoV_N3_FP_LN | GGG AGC CTT GAA TAC ACC AAA A | 78 |
| 2019-nCoV_N3_RP_LN | TGT AGC ACG ATT GCA GCA TTG | 79 |
| 2019-nCoV_N2_FP_LN | TTA CAA ACA TTG GCC GCA AA | 80 |
| 2019-nCoV_N2_RP_LN | GCG CGA CAT TCC GAA GAA | 81 |
| SARS-CoV_FP | ATGTCTGATAATGGACCCCAAA | 82 |
| SARS-CoV_RP | TTAAGCCTGGGTTGAATCAG | 83 |
| MERS-CoV_FP | CACCTCGTGCTGTTTCCTTT | 84 |
| MERS-CoV_RP | ATCATTGGACCAGGCTGAAC | 85 |
| F3_LAMP_N1 | TCATGACGTTCGTGTTGT | 86 |
| B3_LAMP_N1 | TTGAGTGAGAGCGGTGAA | 87 |
| FIP_LAMP_N1 | TAATGCGGGGTGCATTTCGAGATTTCATCTAAACGAA | 88 |
| | CAAAC | |
| BIP_LAMP_N1 | TAACCAGAATGGAGAACGCAAGTATTATTGGGTAAA | 89 |
| | CCTTGG | |
| LF_LAMP_N1 | CTGATTTTGGGGTCCATTA | |
| LB_LAMP_N1 | GTGGGGCGCGATCAAAACAAC | 91 |
| F3_LAMP_N2 | GCTGCTGAGGCTTCTAAG | 92 |
| B3_LAMP_N2 | GCGTCAATATGCTTATTCAGC | 93 |
| FIP_LAMP_N2 | GCGGCCAATGTTTGTAATCAGTAGACGTGGTCCAGA | 94 |
| | ACAA | |
| BIP_LAMP_N2 | TCAGCGTTCTTCGGAATGTCGCTGTGTAGGTCAACCA | 95 |
| | CG | |
| LF_LAMP_N2 | CCTTGTCTGATTAGTTCCTGGT | 96 |
| LB_LAMP_N2 | TGGCATGGAAGTCACACC | 97 |

FQ Substrates and Labeled crRNAs

| | | |
|---|---|---|
| ssDNA-FQ reporterl | /56-FAM/TTATT/3IABkFQ/ | |
| Oligo 2 FAM-Biotin | /56-FAM/TTATT/3Bio/ | |
| 5′Cy5-3′RQ-FQ-Reporter | TAT TA/iCy5/TTAT T/3IAbRQSp/ | 98 |
| F Qreporter-Hex-IowaF Q | /5HEX/TTA TT/3IABkFQ/ | |
| ssDNA-FAM-FQ reporter1 | /56-FAM/TTA TT/3IABkFQ/ | |
| FAM-GC-richFQ-Reporter | /56-FAM/CCG CC/3IABkFQ/ | |
| FQreporter-FAM-ssRNA(rN)-IABFQ | /56-FAM/rCrCrG rCrC/3IABkFQ/ | |
| FQ-reporter-FAM-ssRNA(UArieh) | /56-FAM/rUrUrA rUrU/3IABkFQ/ | |
| LbcrGFP2+3′DNA7-FAM | rUrArA rUrUrU rCrUrA rCrUrA rArGrU | 99 |
| | rGrUrA rGrArU rCrUrC rArGrG rGrCrG | |
| | rGrArC rUrGrG rGrUrG rCrUT ATT | |
| | ATT/36-FAM/ | |
| 5′FAM-LbcrGFP2 | /56-FAM/rUrArA rUrUrU rCrUrA rCrUrA | 100 |
| | rArGrU rGrUrA rGrArU rCrUrC rArGrG | |
| | rGrCrG rGrArC rUrGrG rGrUrG rCrU | |
| LbcrGFP2-3′FAM | rUrArA rUrUrU rCrUrA rCrUrA rArGrU | 101 |
| | rGrUrA rGrArU rCrUrC rArGrG rGrCrG | |
| | rGrArC rUrGrG rGrUrG rCrU/36-FAM/ | |
| 5′FAM-LbcrGFP2+3′DNA13 | /56-FAM/rUrArA rUrUrU rCrUrA rCrUrA | 102 |
| | rArGrU rGrUrA rGrArU rCrUrC rArGrG | |
| | rGrCrG rGrArC rUrGrG rGrUrG rCrUT ATT | |
| | ATT ATT ATT | |
| LbcrGFP2+3′DNA13 -FAM | rUrArA rUrUrU rCrUrA rCrUrA rArGrU | 103 |
| | rGrUrA rGrArU rCrUrC rArGrG rGrCrG | |
| | rGrArC rUrGrG rGrUrG rCrUT ATT ATT | |
| | ATT ATT/36-FAM/ | |
| 5′HEX-DNA19+LbcrGFP2 | /5HEX/TTA TTA TTA TTA TTA TTA | 104 |
| | TrUrA rArUrU rUrCrU rArCrU rArArG | |
| | rUrGrU rArGrA rUrCrU rCrArG rGrGrC | |
| | rGrGrA rCrUrG rGrGrU rGrCrU | |
| LbcrGFP2+DNA-5′FQ-Cy5 | /5IAbRQ/TTA TT/iCy5/A TTA TTA TTA | 105 |
| (crGFP+5′DNA13+Cy5+DNA6+Iowa | TTA TrUrA rArUrU rUrCrU rArCrU rArArG | |
| Black RQ) | rUrGrU rArGrA rUrCrU rCrArG rGrGrC | |
| | rGrGrA rCrUrG rGrGrU rGrCrU | |
| LbcrGFP2+DNA-3′FQ-Cy5 | rUrArA rUrUrU rCrUrA rCrUrA rArGrU | 106 |
| (crGFP+3′DNA7+Cy5+DNA6+Iowa | rGrUrA rGrArU rCrUrC rArGrG rGrCrG | |
| Black RQ) | rGrArC rUrGrG rGrUrG rCrUT ATT ATT | |
| | A/iCy5/TT ATT /3IAbRQSp/ | |
| LbcrGFP2-3′DNA7-Primer-15 | AAT AAT AAG CAC CCA GTC CGC C | 107 |
| LbcrGFP2-3′DNA7-Primer-14 | AAT AAT AAG CAC CCA GTC CGC | 108 |
| 11 | AAT AAT AAG CAC CCA GTC CG | 109 |
| LbcrGFP2-3′DNA7-Primer-12 | AAT AAT AAG CAC CCA GTC C | 110 |
| LbcrGFP2-3′DNA7-Primer-l l | AAT AAT AAG CAC CCA GTC | ill |
| LbcrGFP2-3′DNA7-Primer-10 | AAT AAT AAG CAC CCA GT | 112 |
| LbcrGFP2-3′DNA7-Primer-9 | AAT AAT AAG CAC CCA G | 113 |
| LbcrGFP2-3′DNA7-Primer-8 | AAT AAT AAG CAC CCA | 114 |
| LbcrGFP2-3′DNA7-Primer-7 | AAT AAT AAG CAC CC | 115 |
| LbcrGFP2-3′DNA7-Primer-6 | AAT AAT AAG CAC C | 116 |
| LbcrGFP2-3′DNA7-Primer-5 | AAT AAT AAG CAC | 117 |
| LbcrGFP2-3′DNA7-Primer-4 | AAT AAT AAG CA | 118 |
| LbcrGFP2-Primer-15 | AGC ACC CAG TCC GCC | 119 |
| LbcrGFP2-Primer-14 | AGC ACC CAG TCC GC | 120 |
| LbcrGFP2-Primer-13 | AGC ACC CAG TCC G | 121 |
| LbcrGFP2-Primer-12 | AGC ACC CAG TCC | 122 |
| LbcrGFP2-Primer-11 | AGC ACC CAG TC | 123 |
| LbcrGFP2-Primer-10 | AGC ACC CAG T | 124 |
| LbcrGFP2-Primer-9 | AGC ACC CAG | |
| LbcrGFP2-Primer-8 | AGC ACC CA | |
| RPA-PCA3-FP1 | AGT ACT CAG TGC AGC AAA GAA AGA CTA | 125 |
| | CAG | |
| RPA-PCA3-RP1 | ACA TTA TAT TGT CAT CTT GCT GTT TCT AGT | 126 |
| | GAT | |
| RPA-PCA3-FP2 | AGT GAA GAG CTA GTC CGC TGT GAG TCT | 127 |
| | CCT | |
| RPA-PCA3-RP2 | CTG TTT CTA GTG ATG TTA ATT ATC TCC ATT | 128 |
| | TC | |
| RPA-PCA3-FP3 | AAG AGC TAG TCC GCT GTG AGT CTC CTC | 129 |
| | AGT | |
| RPA-PCA3-RP3 | GTT TCT AGT GAT GTT AAT TAT CTC CAT TTC | 130 |
| | AG | |
| T7-Foward-p rimer 1-RNA | CCT CTA ATA CGA CTC ACT ATA GGA ACG | 131 |
| | GCA TCA AGG TGA ACT | |
| T7-Foward-primer2-RNA | CCT CTA ATA CGA CTC ACT ATA GGC GAC | 132 |
| | CAC TAC CAG CAG AAC A | |
| Primer-EGFP-F490 | ACT TCA AGA TCC GCC ACA AC | 133 |
| Primer-EGFP-F473 | GAA CGG CAT CAA GGT GAA CT | 134 |
| Primer-EGFP-F536 | CGA CCA CTA CCA GCA GAA CA | 135 |

Activators

| | | |
|---|---|---|
| Act-GFP-10nt-T-10nt | CCA CTA CCT GAG CAC CCA GTC CGC CCT | 136 |
| | GAG CAA AGA CCC C | |
| Act-GFP-10nt-NT-10nt | GGG GTC TTT GCT CAG GGC GGA CTG GGT | 137 |
| | GCT CAG GTA GTG G | |
| Act-GFP-10nt-NT-5MeC-10nt | GGG GTC TTT G/iMe-dC/T CAG GGC GGA CTG | 138 |
| | GGT GCT CAG GTA GTG G | |
| Act-GFP-10nt-T-5MeC-10nt | CCA CTA CCT GAG CAC CCA GTC CGC CCT | 139 |
| | GAG /iMe-dC/AA AGA CCC C | |
| Act-GFP-mut-1 | CCA CTA CCT GGG CAC CCA GTC CGC CCT | 140 |
| | GAG CAA AGA CCC C | |
| Act-GFP-mut-2 | CCA CTA CCT GAA CAC CCA GTC CGC CCT | 141 |
| | GAG CAA AGA CCC C | |
| Act-GFP-mut-3 | CCA CTA CCT GAG TAC CCA GTC CGC CCT | 142 |
| | GAG CAA AGA CCC C | |
| Act-GFP-mut-4 | CCA CTA CCT GAG CGC CCA GTC CGC CCT | 143 |
| | GAG CAA AGA CCC C | |
| Act-GFP-mut-5 | CCA CTA CCT GAG CAT CCA GTC CGC CCT | 144 |
| | GAG CAA AGA CCC C | |
| Act-GFP-mut-6 | CCA CTA CCT GAG CAC TCA GTC CGC CCT | 145 |
| | GAG CAA AGA CCC C | |
| Act-GFP-mut-7 | CCA CTA CCT GAG CAC CTA GTC CGC CCT | 146 |
| | GAG CAA AGA CCC C | |
| Act-GFP-mut-8 | CCA CTA CCT GAG CAC CCG GTC CGC CCT | 147 |
| | GAG CAA AGA CCC C | |
| Act-GFP-mut-9 | CCA CTA CCT GAG CAC CCA ATC CGC CCT | 148 |
| | GAG CAA AGA CCC C | |
| Act-GFP-mut-10 | CCA CTA CCT GAG CAC CCA GCC CGC CCT | 149 |
| | GAG CAA AGA CCC C | |
| Act-GFP-mut-11 | CCA CTA CCT GAG CAC CCA GTT CGC CCT | 150 |
| | GAG CAA AGA CCC C | |
| Act-GFP-mut-12 | CCA CTA CCT GAG CAC CCA GTC TGC CCT | 151 |
| | GAG CAA AGA CCC C | |
| Act-GFP-mut-13 | CCA CTA CCT GAG CAC CCA GTC CAC CCT | 152 |
| | GAG CAA AGA CCC C | |
| Act-GFP-mut-14 | CCA CTA CCT GAG CAC CCA GTC CGT CCT | 153 |
| | GAG CAA AGA CCC C | |
| Act-GFP-mut-15 | CCA CTA CCT GAG CAC CCA GTC CGC TCT | 154 |
| | GAG CAA AGA CCC C | |
| Act-GFP-mut-16 | CCA CTA CCT GAG CAC CCA GTC CGC CTT | 155 |
| | GAG CAA AGA CCC C | |
| Act-GFP-mut-17 | CCA CTA CCT GAG CAC CCA GTC CGC CCC | 156 |
| | GAG CAA AGA CCC C | |
| Act-GFP-mut-18 | CCA CTA CCT GAG CAC CCA GTC CGC CCT | 157 |
| | AAG CAA AGA CCC C | |
| Act-GFP-mut-19 | CCA CTA CCT GAG CAC CCA GTC CGC CCT | 158 |
| | GGG CAA AGA CCC C | |
| Act-GFP-mut-20 | CCA CTA CCT GAG CAC CCA GTC CGC CCT | 159 |
| | GAA CAA AGA CCC C | |
| Act-GFP-mut-1-NT | GGG GTC TTT GCT CAG GGC GGA CTG GGT | 160 |
| | GCC CAG GTA GTG G | |
| Act-GFP-mut-2-NT | GGG GTC TTT GCT CAG GGC GGA CTG GGT | 161 |
| | GTT CAG GTA GTG G | |
| Act-GFP-mut-3-NT | GGG GTC TTT GCT CAG GGC GGA CTG GGT | 162 |
| | ACT CAG GTA GTG G | |
| Act-GFP-mut-4-NT | GGG GTC TTT GCT CAG GGC GGA CTG GGC | 163 |
| | GCT CAG GTA GTG G | |
| Act-GFP-mut-5-NT | GGG GTC TTT GCT CAG GGC GGA CTG GAT | 164 |
| | GCT CAG GTA GTG G | |
| Act-GFP-mut-6-NT | GGG GTC TTT GCT CAG GGC GGA CTG AGT | 165 |
| | GCT CAG GTA GTG G | |
| Act-GFP-mut-7-NT | GGG GTC TTT GCT CAG GGC GGA CTA GGT | 166 |
| | GCT CAG GTA GTG G | |
| Act-GFP-mut-8-NT | GGG GTC TTT GCT CAG GGC GGA CCG GGT | 167 |
| | GCT CAG GTA GTG G | |
| Act-GFP-mut-9-NT | GGG GTC TTT GCT CAG GGC GGA TTG GGT | 168 |
| | GCT CAG GTA GTG G | |
| Act-GFP-mut-10-NT | GGG GTC TTT GCT CAG GGC GGG CTG GGT | 169 |
| | GCT CAG GTA GTG G | |
| Act-GFP-mut-11-NT | GGG GTC TTT GCT CAG GGC GAA CTG GGT | 170 |
| | GCT CAG GTA GTG G | |
| Act-GFP-mut-12-NT | GGG GTC TTT GCT CAG GGC AGA CTG GGT | 171 |
| | GCT CAG GTA GTG G | |
| Act-GFP-mut-13-NT | GGG GTC TTT GCT CAG GGT GGA CTG GGT | 172 |
| | GCT CAG GTA GTG G | |
| Act-GFP-mut-14-NT | GGG GTC TTT GCT CAG GAC GGA CTG GGT | 173 |
| | GCT CAG GTA GTG G | |
| Act-GFP-mut-15-NT | GGG GTC TTT GCT CAG AGC GGA CTG GGT | 174 |
| | GCT CAG GTA GTG G | |
| Act-GFP-mut-16-NT | GGG GTC TTT GCT CAA GGC GGA CTG GGT | 175 |
| | GCT CAG GTA GTG G | |
| Act-GFP-mut-17-NT | GGG GTC TTT GCT CGG GGC GGA CTG GGT | 176 |
| | GCT CAG GTA GTG G | |
| Act-GFP-mut-18-NT | GGG GTC TTT GCT TAG GGC GGA CTG GGT | 177 |
| | GCT CAG GTA GTG G | |
| Act-GFP-mut-19-NT | GGG GTC TTT GCC CAG GGC GGA CTG GGT | 178 |
| | GCT CAG GTA GTG G | |
| Act-GFP-mut-20-NT | GGG GTC TTT GTT CAG GGC GGA CTG GGT | 179 |
| | GCT CAG GTA GTG G | |
| Act-GFP-T-2Xmut1 | CCA CTA CCT GGA CAC CCA GTC CGC CCT | 180 |
| | GAG CAA AGA CCC C | |
| Act-GFP-T-2Xmut2 | CCA CTA CCT GAA TAC CCA GTC CGC CCT | 181 |
| | GAG CAA AGA CCC C | |
| Act-GFP-T-2Xmut3 | CCA CTA CCT GAG TGC CCA GTC CGC CCT | 182 |
| | GAG CAA AGA CCC C | |
| Act-GFP-T-2Xmut4 | CCA CTA CCT GAG CGT CCA GTC CGC CCT | 183 |
| | GAG CAA AGA CCC C | |
| Act-GFP-T-2Xmut5 | CCA CTA CCT GAG CAT TCA GTC CGC CCT | 184 |
| | GAG CAA AGA CCC C | |
| Act-GFP-T-2Xmut6 | CCA CTA CCT GAG CAC TTA GTC CGC CCT | 185 |
| | GAG CAA AGA CCC C | |
| Act-GFP-T-2Xmut7 | CCA CTA CCT GAG CAC CTG GTC CGC CCT | 186 |
| | GAG CAA AGA CCC C | |
| Act-GFP-T-2Xmut8 | CCA CTA CCT GAG CAC CCG ATC CGC CCT | 187 |
| | GAG CAA AGA CCC C | |
| Act-GFP-T-2Xmut9 | CCA CTA CCT GAG CAC CCA ACC CGC CCT | 188 |
| | GAG CAA AGA CCC C | |
| Act-GFP-T-2Xmut10 | CCA CTA CCT GAG CAC CCA GCT CGC CCT | 189 |
| | GAG CAA AGA CCC C | |
| Act-GFP-T-2Xmut11 | CCA CTA CCT GAG CAC CCA GTT TGC CCT | 190 |
| | GAG CAA AGA CCC C | |
| Act-GFP-T-2Xmut12 | CCA CTA CCT GAG CAC CCA GTC TAC CCT | 191 |
| | GAG CAA AGA CCC C | |
| Act-GFP-T-2Xmut13 | CCA CTA CCT GAG CAC CCA GTC CAT CCT | 192 |
| | GAG CAA AGA CCC C | |
| Act-GFP-T-2Xmut14 | CCA CTA CCT GAG CAC CCA GTC CGT TCT | 193 |
| | GAG CAA AGA CCC C | |
| Act-GFP-T-2Xmut15 | CCA CTA CCT GAG CAC CCA GTC CGC TIT | 194 |
| | GAG CAA AGA CCC C | |
| Act-GFP-T-2Xmut16 | CCA CTA CCT GAG CAC CCA GTC CGC CTC | 195 |
| | GAG CAA AGA CCC C | |
| Act-GFP-T-2Xmut17 | CCA CTA CCT GAG CAC CCA GTC CGC CCC | 196 |
| | AAG CAA AGA CCC C | |
| Act-GFP-T-2Xmut18 | CCA CTA CCT GAG CAC CCA GTC CGC CCT | 197 |
| | AGG CAA AGA CCC C | |
| Act-GFP-T-2Xmut19 | CCA CTA CCT GAG CAC CCA GTC CGC CCT | 198 |
| | GGA CAA AGA CCC C | |
| Act-GFP-NT-2Xmut1 | GGG GTC TFT GCT CAG GGC GGA CTG GGT | 199 |
| | GTC CAG GTA GTG G | |
| Act-GFP-NT-2Xmut2 | GGG GTC TFT GCT CAG GGC GGA CTG GGT | 200 |
| | ATT CAG GTA GTG G | |
| Act-GFP-NT-2Xmut3 | GGG GTC TFT GCT CAG GGC GGA CTG GGC | 201 |
| | ACT CAG GTA GTG G | |
| Act-GFP-NT-2Xmut4 | GGG GTC TFT GCT CAG GGC GGA CTG GAC | 202 |
| | GCT CAG GTA GTG G | |
| Act-GFP-NT-2Xmut5 | GGG GTC TFT GCT CAG GGC GGA CTG AAT | 203 |
| | GCT CAG GTA GTG G | |
| Act-GFP-NT-2Xmut6 | GGG GTC TFT GCT CAG GGC GGA CTA AGT | 204 |
| | GCT CAG GTA GTG G | |
| Act-GFP-NT-2Xmut7 | GGG GTC TFT GCT CAG GGC GGA CCA GGT | 205 |
| | GCT CAG GTA GTG G | |
| Act-GFP-NT-2Xmut8 | GGG GTC TFT GCT CAG GGC GGA TCG GGT | 206 |
| | GCT CAG GTA GTG G | |
| Act-GFP-NT-2Xmut9 | GGG GTC TTT GCT CAG GGC GGG TTG GGT | 207 |
| | GCT CAG GTA GTG G | |
| Act-GFP-NT-2Xmut10 | GGG GTC TFT GCT CAG GGC GAG CTG GGT | 208 |
| | GCT CAG GTA GTG G | |
| Act-GFP-NT-2Xmut11 | GGG GTC TFT GCT CAG GGC AAA CTG GGT | 209 |
| | GCT CAG GTA GTG G | |
| Act-GFP-NT-2Xmut12 | GGG GTC TFT GCT CAG GGT AGA CTG GGT | 210 |
| | GCT CAG GTA GTG G | |
| Act-GFP-NT-2Xmut13 | GGG GTC TFT GCT CAG GAT GGA CTG GGT | 211 |
| | GCT CAG GTA GTG G | |
| Act-GFP-NT-2Xmut14 | GGG GTC TTT GCT CAG AAC GGA CTG GGT | 212 |
| | GCT CAG GTA GTG G | |
| Act-GFP-NT-2Xmut15 | GGG GTC TFT GCT CAA AGC GGA CTG GGT | 213 |
| | GCT CAG GTA GTG G | |
| Act-GFP-NT-2Xmut16 | GGG GTC TFT GCT CGA GGC GGA CTG GGT | 214 |
| | GCT CAG GTA GTG G | |
| Act-GFP-NT-2Xmut17 | GGG GTC TTT GCT TGG GGC GGA CTG GGT | 215 |
| | GCT CAG GTA GTG G | |
| Act-GFP-NT-2Xmut18 | GGG GTC TFT GCC TAG GGC GGA CTG GGT | 216 |
| | GCT CAG GTA GTG G | |
| Act-GFP-NT-2Xmut19 | GGG GTC TFT GTC CAG GGC GGA CTG GGT | 217 |
| | GCT CAG GTA GTG G | |
| CoV2-Act-T-WT | GGT TAC TGC CAG TTG AAT CTG AGG GTC | 218 |
| | CAC CAA ACG TAA T | |
| CoV2-Act-T-mut1 | GGT TAC TGC CGG TTG AAT CTG AGG GTC | 219 |
| | CAC CAA ACG TAA T | |
| CoV2-Act-T-mut2 | GGT TAC TGC CAA TTG AAT CTG AGG GTC | 220 |
| | CAC CAA ACG TAA T | |
| CoV2-Act-T-mut3 | GGT TAC TGC CAG CTG AAT CTG AGG GTC | 221 |
| | CAC CAA ACG TAA T | |
| CoV2-Act-T-mut4 | GGT TAC TGC CAG TCG AAT CTG AGG GTC | 222 |
| | CAC CAA ACG TAA T | |
| CoV2-Act-T-mut5 | GGT TAC TGC CAG TTA AAT CTG AGG GTC | 223 |
| | CAC CAA ACG TAA T | |
| CoV2-Act-T-mut6 | GGT TAC TGC CAG TTG GAT CTG AGG GTC | 224 |
| | CAC CAA ACG TAA T | |
| CoV2-Act-T-mut7 | GGT TAC TGC CAG TTG AGT CTG AGG GTC | 225 |
| | CAC CAA ACG TAA T | |
| CoV2-Act-T-mut8 | GGT TAC TGC CAG TTG AAC CTG AGG GTC | 226 |
| | CAC CAA ACG TAA T | |
| CoV2-Act-T-mut9 | GGT TAC TGC CAG TTG AAT TTG AGG GTC | 227 |
| | CAC CAA ACG TAA T | |
| CoV2-Act-T-mut10 | GGT TAC TGC CAG TTG AAT CCG AGG GTC | 228 |
| | CAC CAA ACG TAA T | |
| CoV2-Act-T-mut11 | GGT TAC TGC CAG TTG AAT CTA AGG GTC | 229 |
| | CAC CAA ACG TAA T | |
| CoV2-Act-T-mut12 | GGT TAC TGC CAG TTG AAT CTG GGG GTC | 230 |
| | CAC CAA ACG TAA T | |
| CoV2-Act-T-mut13 | GGT TAC TGC CAG TTG AAT CTG AAG GTC | 231 |
| | CAC CAA ACG TAA T | |
| CoV2-Act-T-mut14 | GGT TAC TGC CAG TTG AAT CTG AGA GTC | 232 |
| | CAC CAA ACG TAA T | |
| CoV2-Act-T-mut15 | GGT TAC TGC CAG TTG AAT CTG AGG ATC | 233 |
| | CAC CAA ACG TAA T | |
| CoV2-Act-T-mut16 | GGT TAC TGC CAG TTG AAT CTG AGG GCC | 234 |
| | CAC CAA ACG TAA T | |
| CoV2-Act-T-mut17 | GGT TAC TGC CAG TTG AAT CTG AGG GTT | 235 |
| | CAC CAA ACG TAA T | |
| CoV2-Act-T-mut18 | GGT TAC TGC CAG TTG AAT CTG AGG GTC | 236 |
| | TAC CAA ACG TAA T | |
| CoV2-Act-T-mut19 | GGT TAC TGC CAG TTG AAT CTG AGG GTC | 237 |
| | CGC CAA ACG TAA T | |
| CoV2-Act-T-mut20 | GGT TAC TGC CAG TTG AAT CTG AGG GTC | 238 |
| | CAT CAA ACG TAA T | |
| CoV2-Act-NT-WT | ATT ACG TTT GGT GGA CCC TCA GAT TCA | 239 |
| | ACT GGC AGT AAC C | |
| CoV2-Act-NT-mut1 | ATT ACG TTT GGT GGA CCC TCA GAT TCA | 240 |
| | ACC GGC AGTAACC | |
| CoV2-Act-NT-mut2 | ATT ACG TTT GGT GGA CCC TCA GAT TCA | 241 |
| | ATT GGC AGTAACC | |
| CoV2-Act-NT-mut3 | ATT ACG TTT GGT GGA CCC TCA GAT TCA | 242 |
| | GCT GGC AGTAACC | |
| CoV2-Act-NT-mut4 | ATT ACG TTT GGT GGA CCC TCA GAT TCG | 243 |
| | ACT GGC AGTAACC | |
| CoV2-Act-NT-mut5 | ATT ACG TTT GGT GGA CCC TCA GAT TTA | 244 |
| | ACT GGC AGTAACC | |
| CoV2-Act-NT-mut6 | ATT ACG TTT GGT GGA CCC TCA GAT CCA | 245 |
| | ACT GGC AGTAACC | |
| CoV2-Act-NT-mut7 | ATT ACG TTT GGT GGA CCC TCA GAC TCA | 246 |
| | ACT GGC AGTAACC | |
| CoV2-Act-NT-mut8 | ATT ACG TTT GGT GGA CCC TCA GGT TCA | 247 |
| | ACT GGC AGTAACC | |
| CoV2-Act-NT-mut9 | ATT ACG TTT GGT GGA CCC TCA AAT TCA | 248 |
| | ACT GGC AGTAACC | |
| CoV2-Act-NT-mut10 | ATT ACG TTT GGT GGA CCC TCG GAT TCA | 249 |
| | ACT GGC AGTAACC | |
| CoV2-Act-NT-mut11 | ATT ACG TTT GGT GGA CCC TTA GAT TCA | 250 |
| | ACT GGC AGTAACC | |
| CoV2-Act-NT-mut12 | ATT ACG TTT GGT GGA CCC CCA GAT TCA | 251 |
| | ACT GGC AGTAACC | |
| CoV2-Act-NT-mut13 | ATT ACG TTT GGT GGA CCT TCA GAT TCA | 252 |
| | ACT GGC AGTAACC | |
| CoV2-Act-NT-mut14 | ATT ACG TTT GGT GGA CTC TCA GAT TCA | 253 |
| | ACT GGC AGTAACC | |
| CoV2-Act-NT-mut15 | ATT ACG TTT GGT GGA TCC TCA GAT TCA | 254 |
| | ACT GGC AGTAACC | |
| CoV2-Act-NT-mut16 | ATT ACG TTT GGT GGG CCC TCA GAT TCA | 255 |
| | ACT GGC AGTAACC | |
| CoV2-Act-NT-mut17 | ATT ACG TTT GGT GAA CCC TCA GAT TCA | 256 |
| | ACTGGC AGTAACC | |
| CoV2-Act-NT-mut18 | ATT ACG TTT GGT AGA CCC TCA GAT TCA | 257 |
| | ACT GGC AGTAACC | |
| CoV2-Act-NT-mut19 | ATT ACG TTT GGC GGA CCC TCA GAT TCA | 258 |
| | ACT GGC AGTAACC | |
| CoV2-Act-NT-mut20 | ATT ACG TTT GAT GGA CCC TCA GAT TCA | 259 |
| | ACT GGC AGTAACC | |
| CoV2-Act-T-2Xmut1 | GGT TAC TGC CGA TTG AAT CTG AGG GTC | 260 |
| | CAC CAA ACG TAA T | |
| CoV2-Act-T-2Xmut2 | GGT TAC TGC CAA CTG AAT CTG AGG GTC | 261 |
| | CAC CAA ACG TAA T | |
| CoV2-Act-T-2Xmut3 | GGT TAC TGC CAG CCG AAT CTG AGG GTC | 262 |
| | CAC CAA ACG TAA T | |
| CoV2-Act-T-2Xmut4 | GGT TAC TGC CAG TCA AAT CTG AGG GTC | 263 |
| | CAC CAA ACG TAA T | |
| CoV2-Act-T-2Xmut5 | GGT TAC TGC CAG TTA GAT CTG AGG GTC | 264 |
| | CAC CAA ACG TAA T | |
| CoV2-Act-T-2Xmut6 | GGT TAC TGC CAG TTG GGT CTG AGG GTC | 265 |
| | CAC CAA ACG TAA T | |
| CoV2-Act-T-2Xmut7 | GGT TAC TGC CAG TTG AGC CTG AGG GTC | 266 |
| | CAC CAA ACG TAA T | |
| CoV2-Act-T-2Xmut8 | GGT TAC TGC CAG TTG AAC TTG AGG GTC | 267 |
| | CAC CAA ACG TAA T | |
| CoV2-Act-T-2Xmut9 | GGT TAC TGC CAG TTG AAT TCG AGG GTC | 268 |
| | CAC CAA ACG TAA T | |
| CoV2-Act-T-2Xmut10 | GGT TAC TGC CAG TTG AAT CCA AGG GTC | 269 |
| | CAC CAA ACG TAA T | |
| CoV2-Act-T-2Xmut11 | GGT TAC TGC CAG TTG AAT CTA GGG GTC | 270 |
| | CAC CAA ACG TAA T | |
| CoV2-Act-T-2Xmut12 | GGT TAC TGC CAG TTG AAT CTG GAG GTC | 271 |
| | CAC CAA ACG TAA T | |
| CoV2-Act-T-2Xmut13 | GGT TAC TGC CAG TTG AAT CTG AAA GTC | 272 |
| | CAC CAA ACG TAA T | |
| CoV2-Act-T-2Xmut14 | GGT TAC TGC CAG TTG AAT CTG AGA ATC | 273 |
| | CAC CAA ACG TAA T | |
| CoV2-Act-T-2Xmut15 | GGT TAC TGC CAG TTG AAT CTG AGG ACC | 274 |
| | CAC CAA ACG TAA T | |
| CoV2-Act-T-2Xmut16 | GGT TAC TGC CAG TTG AAT CTG AGG GCT | 275 |
| | CAC CAA ACG TAA T | |
| CoV2-Act-T-2Xmut17 | GGT TAC TGC CAG TTG AAT CTG AGG GTT | 276 |
| | TAC CAA ACG TAA T | |
| CoV2-Act-T-2Xmut18 | GGT TAC TGC CAG TTG AAT CTG AGG GTC | 277 |
| | TGC CAA ACG TAA T | |
| CoV2-Act-T-2Xmut19 | GGT TAC TGC CAG TTG AAT CTG AGG GTC | 278 |
| | CGT CAA ACG TAA T | |
| CoV2-Act-NT-2Xmut1 | ATT ACG TTT GGT GGA CCC TCA GAT TCA | 279 |
| | ATC GGC AGT AAC C | |
| CoV2-Act-NT-2Xmut2 | ATT ACG TTT GGT GGA CCC TCA GAT TCA | 280 |
| | GTT GGC AGT AAC C | |
| CoV2-Act-NT-2Xmut3 | ATT ACG TTT GGT GGA CCC TCA GAT TCG | 281 |
| | GCT GGC AGT AAC C | |
| CoV2-Act-NT-2Xmut4 | ATT ACG TTT GGT GGA CCC TCA GAT TTG | 282 |
| | ACT GGC AGT AAC C | |
| CoV2-Act-NT-2Xmut5 | ATT ACG TTT GGT GGA CCC TCA GAT CTA | 283 |
| | ACT GGC AGT AAC C | |
| CoV2-Act-NT-2Xmut6 | ATT ACG TTT GGT GGA CCC TCA GAC CCA | 284 |
| | ACT GGC AGT AAC C | |
| CoV2-Act-NT-2Xmut7 | ATT ACG TTT GGT GGA CCC TCA GGC TCA | 285 |
| | ACT GGC AGT AAC C | |
| CoV2-Act-NT-2Xmut8 | ATT ACG TTT GGT GGA CCC TCA AGT TCA | 286 |
| | ACT GGC AGT AAC C | |
| CoV2-Act-NT-2Xmut9 | ATT ACG TTT GGT GGA CCC TCG AAT TCA | 287 |
| | ACT GGC AGT AAC C | |
| CoV2-Act-NT-2Xmut10 | ATT ACG TTT GGT GGA CCC TTG GAT TCA | 288 |
| | ACT GGC AGT AAC C | |
| CoV2-Act-NT-2Xmut11 | ATT ACG TTT GGT GGA CCC CTA GAT TCA | 289 |
| | ACT GGC AGT AAC C | |
| CoV2-Act-NT-2Xmut12 | ATT ACG TTT GGT GGA CCT CCA GAT TCA | 290 |
| | ACT GGC AGT AAC C | |
| CoV2-Act-NT-2Xmut13 | ATT ACG TTT GGT GGA CTT TCA GAT TCA ACT | 291 |
| | GGC AGT AAC C | |
| CoV2-Act-NT-2Xmut14 | ATT ACG TTT GGT GGA TTC TCA GAT TCA ACT | 292 |
| | GGC AGT AAC C | |
| CoV2-Act-NT-2Xmut15 | ATT ACG TTT GGT GGG TCC TCA GAT TCA | 293 |
| | ACT GGC AGT AAC C | |
| CoV2-Act-NT-2Xmut16 | ATT ACG TTT GGT GAG CCC TCA GAT TCA | 294 |
| | ACT GGC AGT AAC C | |
| CoV2-Act-NT-2Xmut17 | ATT ACG TTT GGT AAA CCC TCA GAT TCA | 295 |
| | ACT GGC AGT AAC C | |
| CoV2-Act-NT-2Xmut18 | ATT ACG TTT GGC AGA CCC TCA GAT TCA | 296 |
| | ACT GGC AGT AAC C | |
| CoV2-Act-NT-2Xmut19 | ATT ACG TTT GAC GGA CCC TCA GAT TCA | 297 |
| | ACT GGC AGT AAC C | |

(“*” refers to phosphorothioate nucleotide)

1. Chen, J. S. et al. CRISPR-Cas12a target binding unleashes indiscriminate single-stranded DNase activity. Science 360, 436-439 (2018).
2. Wang, Q. et al. The CRISPR-Cas13a Gene-Editing System Induces Collateral Cleavage of RNA in Glioma Cells. Adv Sci 6, 1901299 (2019).
3. Li, S. Y. et al. CRISPR-Cas12a has both cis- and trans-cleavage activities on single-stranded DNA. Cell Res 28, 491-493 (2018).
4. Gootenberg, J. S. et al. Nucleic acid detection with CRISPR-Cas13a/C2c2. Science 356, 438-442 (2017).
5. Gootenberg, J. S. et al. Multiplexed and portable nucleic acid detection platform with Cas13, Cas12a, and Csm6. Science 360, 439-444 (2018).
6. Harrington, L. B. et al. Programmed DNA destruction by miniature CRISPR-Cas14 enzymes. Science 362, 839-842 (2018).
7. Kellner, M. J., Koob, J. G., Gootenberg, J. S., Abudayyeh, O. O. & Zhang, F. SHERLOCK: nucleic acid detection with CRISPR nucleases. Nat Protoc 14, 2986-3012 (2019).
8. Abudayyeh, O. O., Gootenberg, J. S., Kellner, M. J. & Zhang, F. Nucleic Acid Detection of Plant Genes Using CRISPR-Cas13. CRISPR J 2, 165-171 (2019).
9. Kocak, D. D. et al. Increasing the specificity of CRISPR systems with engineered RNA secondary structures. Nat Biotechnol 37, 657-666 (2019).
10. Park, H. M. et al. Extension of the crRNA enhances Cpf1 gene editing in vitro and in vivo. Nat Commun 9, 3313 (2018).
11. McMahon, M. A., Prakash, T. P., Cleveland, D. W., Bennett, C. F. & Randar, M. Chemically Modified Cpf1-CRISPR RNAs Mediate Efficient Genome Editing in Mammalian Cells. Mol Ther 26, 1228-1240 (2018).
12. Yamano, T. et al. Structural Basis for the Canonical and Non-canonical PAM Recognition by CRISPR-Cpf1. Mol Cell 67, 633-645.e633 (2017).
13. Fuchs, R. T., Curcuru, J., Mabuchi, M., Yourik, P. & Robb, G. B. Cas12a trans-cleavage can be modulated in vitro and is active on ssDNA, dsDNA, and RNA. (2019).
14. Li, B. et al. Synthetic Oligonucleotides Inhibit CRISPR-Cpf1-Mediated Genome Editing. Cell Rep 25, 3262-3272.e3263 (2018).
15. Yamano, T. et al. Crystal Structure of Cpf1 in Complex with Guide RNA and Target DNA. Cell 165, 949-962 (2016).
16. Dong, D. et al. The crystal structure of Cpf1 in complex with CRISPR RNA. Nature 532, 522-526 (2016).
17. Swarts, D. C., van der Oost, J. & Jinek, M. Structural Basis for Guide RNA Processing and Seed-Dependent DNA Targeting by CRISPR-Cas12a. Mol Cell 66, 221-233.e224 (2017).
18. Dai, Y. et al. Exploring the Trans-Cleavage Activity of CRISPR-Cas12a (cpf1) for the Development of a Universal Electrochemical Biosensor. Angew Chem Int Ed Engl 58, 17399-17405 (2019).
19. Zetsche, B. et al. Cpf1 is a single RNA-guided endonuclease of a class 2 CRISPR-Cas system. Cell 163, 759-771 (2015).
20. Fonfara, I., Richter, H., Bratovič, M., Le Rhun, A. & Charpentier, E. The CRISPR-associated DNA-cleaving enzyme Cpf1 also processes precursor CRISPR RNA. Nature 532, 517-521 (2016).
21. Marks, L. S. & Bostwick, D. G. Prostate Cancer Specificity of PCA3 Gene Testing: Examples from Clinical Practice. Rev Urol 10, 175-181 (2008).
22. Laxman, B. et al. A first-generation multiplex biomarker analysis of urine for the early detection of prostate cancer. Cancer Res 68, 645-649 (2008).
23. Yang, Z., Yu, L. & Wang, Z. PCA3 and TMPRSS2-ERG gene fusions as diagnostic biomarkers for prostate cancer. Chin J Cancer Res 28, 65-71 (2016).
24. Filella, X. et al. PCA3 in the detection and management of early prostate cancer. Tumour Biol 34, 1337-1347 (2013).
25. Ferro, M. et al. Prostate Health Index (Phi) and Prostate Cancer Antigen 3 (PCA3) significantly improve prostate cancer detection at initial biopsy in a total PSA range of 2-10 ng/ml. PLoS One 8, e67687 (2013).
26. Loeb, S. & Partin, A. W. PCA3 Urinary Biomarker for Prostate Cancer. Rev Urol 12, e205-206 (2010).
27. Zhang, F., Abudayyeh, O. O., Gootenberg, J. S., Sciences, C. & Mathers, L. A protocol for detection of COVID-19 using CRISPR diagnostics v.20200321. Sherlock Biosciences, Broad Institute, MIT: Cambridge, Mass. (2020).
28. Broughton, J. P. et al. A protocol for rapid detection of the 2019 novel coronavirus SARSCoV-2 using CRISPR diagnostics: SARS-CoV-2 DETECTR v3. Mammoth Biosciences, South San Francisco, Calif. (2020).
29. Goujon, M. et al. A new bioinformatics analysis tools framework at EMBL-EBI. Nucleic Acids Res 38, W695-699 (2010).
30. Sievers, F. et al. Fast, scalable generation of high-quality protein multiple sequence alignments using Clustal Omega. Mol Syst Biol 7, 539 (2011).
31. Robert, X. & Gouet, P. Deciphering key features in protein structures with the new ENDscript server. Nucleic Acids Res 42, W320-324 (2014).
32. Moreno-Mateos, M. A. et al. CRISPR-Cpf1 mediates efficient homology-directed repair and temperature-controlled genome editing. Nat Commun 8, 2024 (2017).
33. Hajian, R. et al. Detection of unamplified target genes via CRISPR-Cas9 immobilized on a graphene field-effect transistor. Nat. Biomed Eng 3, 427-437 (2019).
34. Notredame, C., Higgins, D. G. & Heringa, J. T-Coffee: A novel method for fast and accurate multiple sequence alignment. J Mol Biol 302, 205-217, (2000).

1. A nucleic acid detection system comprising:
a CRISPR-associated (Cas) enzyme with trans cleavage activity;
a guide CRISPR RNA (crRNA) comprising a guide sequence and a polynucleotide extension sequence, wherein the guide sequence is configured to bind to a target polynucleotide, the polynucleotide extension sequence is linked to a 3′-end of the guide sequence, and the polynucleotide extension sequence comprises a ssDNA or ssRNA having 1-19 nucleotides; and
a plurality of probes, each probe comprising an oligonucleotide element labeled with a detectable label, wherein the probe is configured to be cleaved by the Cas enzyme when the guide sequence binds the target polynucleotide to generate a detectable signal or a detectable molecule.

2. The detection system of claim 1, wherein a first end of the oligonucleotide element in the probe is linked to a fluorophore; a second end of the oligonucleotide element in the probe is linked to a quencher such that the fluorophore produces a detectable signal upon cleavage of the oligonucleotide element to release the quencher.

3. The detection system of claim 1, wherein the polynucleotide extension sequence comprises 7 nucleotides.

4. The detection system of claim 1, wherein the polynucleotide extension sequence is a ssDNA.

5. The detection system of claim 4, wherein the ssDNA comprises at least at least 80% of A and/or T.

6. The detection system of claim 5, wherein the ssDNA comprises a nucleotide sequence of TATTATT.

7. The detection system of claim 1, wherein the oligonucleotide element in the probe is a ssDNA or RNA.

8. The detection system of claim 7, wherein the oligonucleotide element in the probe is a ssDNA, and the ssDNA in the probe comprises a nucleotide sequence of TTATT.

9. The detection system of claim 1, wherein the probe comprises FAM-TTATT-3IABkFQ or FAM-TTATTA(internal biotin)T-3IABkFQ.

10. The detection system of claim 1, wherein the CRISPR-associated enzyme is selected from the group consisting of MbCas12a, FnCas12a and LbCas12a.

11. The detection system of any one of the proceeding claims, wherein the target polynucleotide is a ssDNA, a dsDNA, a methylated DNA, a methylated RNA, or a heteroduplex of RNA and DNA.

12. The detection system of claim 1, further comprising a nucleic acid amplification reagent for an amplification selected from the group consisting of recombinase polymerase amplification (RPA), and loop-mediated isothermal amplification (LAMP).

13. The detection system of claim 1, wherein the guide sequence is complementary to a target polynucleotide that is diagnostic for a disease state selected from the group consisting of cancer, a genetic disease or disorder, and an infection.

14. The detection system of claim 13, wherein the disease state is an infection caused by a virus selected from the group consisting of: SARS-CoV-2, HIV, and HCV.

15. The detection system of claim 14, wherein the system comprises two guide CRISPR RNAs (crRNAs): a first crRNA comprising a first guide sequence configured to bind to a first target polynucleotide, wherein the first target polynucleotide is a DNA fragment synthesized from a RNA fragment in an N1 region of SARS-CoV-2; and a second crRNA comprising a second guide sequence configured to bind to a second target polynucleotide, wherein the second target polynucleotide is a DNA fragment synthesized from a RNA fragment in an N2 region of SARS-CoV-2.

16. The detection system of claim 1, further comprising a divalent metal cation; optionally the divalent metal cation is magnesium.

17. The detection system of claim 1, wherein the crRNA further comprises a linker having two ends, one end of the linker linked to 3′-end of the polynucleotide extension sequence, and the other end of the linker linked to a complementary sequence, wherein the complementary sequence is complementary to the extension sequence, wherein the extension sequence, the linker, and the complementary sequence form a toehold conformation, and the toehold conformation unfolds when the guide sequence binds to the target polynucleotide.

18. The nucleic acid detection system of claim 1, wherein the polynucleotide extension sequence comprises a linker having two ends, one end of the linker linked to 3′-end of the guide sequence, and the other end of the linker linked to a complementary sequence, wherein the complementary sequence is complementary to at least a 3′ portion of the guide sequence, wherein the linker, the complementary sequence, and the 3′ portion of the guide sequence form a toehold conformation, and the toehold conformation unfolds when the guide sequence binds to the target polynucleotide.

19-21. (canceled)

22. A method of detecting a target polynucleotide in a sample obtained from a subject, the method comprising:
contacting the sample with the detection system of any one of claims 1-18, wherein the guide sequence is substantially complementary to the target polynucleotide such that the guide sequence preferentially binds the target polynucleotide, and wherein hybridizing the guide sequence and the target polynucleotide leads to activating the CRISPR-associated enzyme which results in cleavage of the probe such that a detectable signal or a detectable molecule is produced; and
detecting the signal or the molecule, wherein detection of the signal or the molecule indicates presence of the target polynucleotide in the sample.

23. (canceled)

24. The method of claim 22, wherein the sample and the detection system are incubated at a temperature of 40-60° C. for a period of time.

25. (canceled)

26. The method of claim 22, further comprising amplifying the target polynucleotide in the sample prior to contacting the sample with the detection system.

27. (canceled)
